SEQUENCE LISTING (46 sequences)

SEQ ID NO: 1 - DNA, 25 bp, Mus musculus
  source 1..25: mol_type = genomic DNA
acctgctgtgaaaagaaaaagccga

SEQ ID NO: 2 - DNA, 25 bp, Homo sapiens
  source 1..25: mol_type = genomic DNA
atatcaatttggtccttgcaccaca

SEQ ID NO: 3 - DNA, 25 bp, Homo sapiens
  source 1..25: mol_type = genomic DNA
acctagcaatgtttcaattactgtc

SEQ ID NO: 4 - DNA, 25 bp, Homo sapiens
  source 1..25: mol_type = genomic DNA
atacatggaaacacatacccatttt

SEQ ID NO: 5 - DNA, 28 bp, Homo sapiens
  source 1..28: mol_type = genomic DNA
caaatacatggaaacacatacccatttt

SEQ ID NO: 6 - DNA, 30 bp, Homo sapiens
  source 1..30: mol_type = genomic DNA
tgcaaatacatggaaacacatacccatttt

SEQ ID NO: 7 - DNA, 28 bp, Homo sapiens
  source 1..28: mol_type = genomic DNA
atacatggaaacacatacccatttttcc

SEQ ID NO: 8 - DNA, 30 bp, Homo sapiens
  source 1..30: mol_type = genomic DNA
atacatggaaacacatacccatttttccac

SEQ ID NO: 9 - DNA, 25 bp, Homo sapiens
  source 1..25: mol_type = genomic DNA
aacttgctgtggaaagaaaaagcca

SEQ ID NO: 10 - DNA, 28 bp, Homo sapiens
  source 1..28: mol_type = genomic DNA
atcaacttgctgtggaaagaaaaagcca

SEQ ID NO: 11 - DNA, 30 bp, Homo sapiens
  source 1..30: mol_type = genomic DNA
acatcaacttgctgtggaaagaaaaagcca

SEQ ID NO: 12 - DNA, 28 bp, Homo sapiens
  source 1..28: mol_type = genomic DNA
aacttgctgtggaaagaaaaagccaaaa

SEQ ID NO: 13 - DNA, 30 bp, Homo sapiens
  source 1..30: mol_type = genomic DNA
aacttgctgtggaaagaaaaagccaaaata

SEQ ID NO: 14 - DNA, 25 bp, Homo sapiens
  source 1..25: mol_type = genomic DNA
acttttagatcacaatttctttgca

SEQ ID NO: 15 - DNA, 25 bp, Homo sapiens
  source 1..25: mol_type = genomic DNA
gattagaggcgagagcaagcatgca

SEQ ID NO: 16 - DNA, 25 bp, Homo sapiens
  source 1..25: mol_type = genomic DNA
atagtacaagccacactaggaagca

SEQ ID NO: 17 - DNA, 25 bp, Homo sapiens
  source 1..25: mol_type = genomic DNA
tgttcattacaacgaagcagttagt

SEQ ID NO: 18 - DNA, 25 bp, Homo sapiens
  source 1..25: mol_type = genomic DNA
attgtcttcttctacagtacatgca

SEQ ID NO: 19 - DNA, 33375 bp, Homo sapiens
  source 1..33375: mol_type = genomic DNA
gcaagtacccaatttatgtagacttgtagtattttaatgagctcagctactataggaacaatttctttcacaggtgtcagagattttctttttgctaaaataagtccatgcctttcagttggaatgtccttaggtttgactttctgctttatctagcatatttgtggtgtcttgaaaactaaaaaaatatatccaagcttgatgtgatctgtgtcactaggcctcgtgcttatgtctgttgtttaaaaccgacctttctttgagcgtttctgtgtttcattacattttcagagctgttattgttattattttaactcatagctttatgcatggctatttgtcattaacctttatttcattttcaaatgtctgtcactatgtgactatgttctgagctttttcatggaaagtaaaatttcatggcactgttatatattttaagtaaccaaattttggtgatatggctttaggctattgtaattttgcaaacagctaatccttctcctctgaaaattgaaatttttcttttcttttttttgagatggagtctggctctgtcacccaggctggagtgcagtggcgcgatctcagctcactgcaagctctgcctcccgggttcatgccattctcctgcctcagcctcctgagtagctgggactacaggctcctgccaccaccacacctggctattttttttttttttttctatttttagtagagacggggtttcaccatgttggccaggatggtcttgacctcctgacctcgtgatctgcccaccttggcctcccaaagtgctgggattacaggcgtgagccaccgcgcccagccctgaaattgttatgtggtttcctaatgcaagtatttttctttcaaagcctatttggttattttattttcaatgagacttcctgtgactgatatctgggctgttacccagtcttaaccaatttattaaagtttagaattaagcaggaggattaatggtcaaattttttgtttccaggttttttaatttttattttttggtatggtaattactgcagtgtatggtttaataaagtgtagtatttccagagagaaaactgaaaattttattttgccattattgatctatatgttgctgttgttattaatcattactctggtctggataaataaagagattaaaaaatggtaatatataaatgattatccatagaaaggagtgatcagttataacgttattttcatcagccttgtagtttttaaaattttagtgtcatgcagcttatgactttagttgatttgtgcattaattctaaattaaatttcagcagtgtactattaagtagggaagagaaactttagtgacttttgatattagactagaagcttattttacaaaagcttagtattttagtttttcaggttcattttttgtttacaacaaagtttgaaattattaaaatatataaagtttcttttaaaaatttctattctttcatgtaaaccttctcagttatgcttaagttatgaaagagtaaaacaattctcaatttagtttcatttctacctgaaataacccctcctgatttttaaacatccctgagtcatatcaactttgatatgcaaatcagcttttataaagaaagatcatttttatttggcattagcatttaaaaaatgcatcttgccatttcatctgtctttatagataacaacttagaactaaattgccataaatttaatttttttattgttaagttgcgttttttaaatttaacaactttgagaagatcttttatcttcttcaaatcatgaggttgggctgggcaaattctaaagtttcttgaaatataggaagtaatttgaaatactgttttaggatttatggtttttttgagatggggtcttgctatatggcccaggctggtcttgagctcctgagctcaaacaatcctcctgcctcagtctctggaatatctaggattacagatgcatgccacagtgtacccagcttctaggatttatgttttcaagacatttattagttagaatttctggatctcctcaagtatttttatttagctgaatttagttttgaattgttcactctctagaatgtctccatcacagataaagtagtaatttgtgtgatgtgttgaacactattttataaagcgcagtatagttaccataagacatgaattaggaagtataatttgaggaaaaactgctacaaggaataaacacattgcatgaagctgaaacctatttctggttattagtaacttttagaaagaaaccaatgtcaaatagctcaacggggtaccgtatattagggaaaaatgaaagtttctctcatactgctgagtatttttgattatacccagaaatgatattcattcctagcttgccacaggtgatgccagttaatgagcacagccccagccacagcgttggaggaggagttctggaggcctctattgtatttatggatttggaggtagcccgagcaacggggggtgtcctggaggacccctggggtggtcagggttagggagcactgtggggacttgcggcagcaactgtttaccaagaaggatagatattttccacttaaccattttattcataaggatgtgcaccagctagatatcatgtctgatgatatgtgactcatggatgctgctggcacttgtaggaaccaagtcttctgaactccaaatatcatcttttctagtgccttataatgtcccctttacactcattatggaactctaatggggaatttttttatcaatcaaattactatctccaggttaataaatcatgtgtagtttgcttaaatgaagagccacttttcctaggatgaagtgtgtcaggaaggtaatgtgaattttcaagaaatcatttctttcctttcagatgaacagtgagtcaaaaagaacctcgtgttgccctgattcccaactctttcataatcaactttctttacgaagccctgttcttttttatttaataattcttctttaatttcttacgtaattcagtaaattaaacaatacttatactatttcatgtccaaaatgttatatgcacatagcaatacaaacacacttgatgataaggaaaatttttccatttagaaccatgcctttgttttttgtggttaaggaccattttgctagtattcttgggtcatgaataaatacatggttttcataaaatggttaaggaaacttagcagaaaactcccaatgtcatttgtgacaaagtaaatcatgaacattacgataacagaagaagatgtgtaagatgaaatgggagaaaatattacctaatgtatgaagttttggttccctaaaaagttgcttattgtgactattagtaaaataactcatatttttgtggcaccaaaatcattctgaaggatttcatctaagtggattttctaaaatgtctgtttaattaaaatatatatatatatatatatgttaaaagagagacctagatccagtatttatataattacattgactatcatttagcttttattcttattaaaatcatgtcaatagtaaataggccaagacctgtctatctcagtagctgcccacagaagctttcagatcttgaggatggagacagtacacaggcaacgagaaggagaaggtgtctgttgagcagcaaccatctctttggccccgttttctctgggcgtctgcctatgcacattctggcttctgctgttctttctatttcccccactgggttttctgtggttctggataacttgtcgcttgcagatctacctctgtattgctcttcttgctcttcagatctattttgcgtatggatgatatagcatagggttaaaaacaggggtttattgtcttagagaactgcttgagaataccagttctgccatagttagctctgtggctttgggaaagataactttatcatcaaggcttagtaattattaaatgatggtgcatgttattattgttttatttattcttttttgtatacttttaaactccctgggttggactgctattaattcctgactgccttttctctgggcttgatggctttcttccttctctgggacatagccagtccagttcccacttcctcccctcgaccccagtattctcatcaatcctggcacatgtagtttgccgatttaatgatgccgcagcagtgttagaatagagagttgatgatagcatttgcttgtttccatgtgttttgtagttaataaagtttcataatttaagtaatattgtgcttttggctattccttttatcttaattcacaaaattatgatagacacttttctctcacacaactttatttgtaatgccaaagttaatttaaggatttcatctcctatgagcacagatatcaagtgagaggtgacgtagtatagtagaaagggctttggttttggagtccaggaaatttactgaaagttctttttcaaataatatttataataattatcatctgttgaaaaggcgttgctttggtttaatgccatgagtccaatatatggcttgtatattctgttgaggacagcgtattttaaaatacacgttgagtgtcctttctgtctttcgggtgaattatttctggggggattgtaggaccctgtccttttcaactttagcttatggccacttgactgggtccctttattattagaatattgtagattgaactagagaaaaaattaaaattaatcattctttgctccttacatcgtgctctttgatggagtaatagattaacatttataattcaggtgattatgatttggatcattcattcatttgctatataatcctcatgatcgtgggtattggtagtttaccgctatgtaagttatggatgatactttaaaaatctataaaattcacaatgtaatgttataaatattgtgatgagcaaataatattgaaatgaatattaaataaatccaattctaatcatttggtgctttttcaacatttataaggtaacttatatcttataaaggttctatgtatagaaatttaagtgtttttttgtcatcaatgagtttacaactgcagggatagaaaaacaattatagtataatttcatgtatgttatagaagtatatataattatcatgttggcatgaagaaggaattgtgtacgtggaacttgttgttcctttacattttcttggcactttttagaatcatttccaaaactattagaagtaatattaaaactattaatagaaaaatcatttcaaaattttttccaaggaacaatttgagctcattaggcaattccttaaaattttaatagaaaataatacttgtcaggaagacaatccaaaacatgtccagagatattcttaaaatgatttttatgtttcagtttagtaaaatggcttattgttttataagaaactgtaatgatgtttcctctttctaaattatgtactgtcttcaaagtacaaaatattacaatgacttaattattaacagaggtatagatcagactcagacatgtccagcttttatgaaaaatggagctgtagaatgtggagtagtaaaaaacttgtgttggctgggcacggtggctcacgcctgtaatcccagcactttgggaggccgaggtgggtggatcacttgaggtcaggagtttgagaccagcctggccaatgtggagaaaccccgtctcttatgaaaaatacaaaaattagctgggtgtggtggcgcacacctgtaatcccagctactcgggaggctgaagcaagagaatcgcttgaacccaggagatggaggttgtagagagcagagattgcaccactgttctccagcctgggcaacaaagtgagactccatctctaaataaataaataaattaattaattaattaaataaaacacttgtgtcattctttttataaaacccctatctctttaagacatgtactctaagaacattcaaaaatgcatggggctgggcacggtggctcacacctgtcatcccagcaatttgggaggctgaggcatgtggatcacttgaggtcaggaattcgagaccagcctagccaacatgttgaaaccccatctctactaaaaccacaaaaaattagccagacgtggtgacacgtgcctgtaatcccagctacttgggaggctgaggcacgagaatcacttgaacccgggaggcggattttgcagtgagcccagatcgtgccactgcactccagcctgggcaacagagcgagtatccatctcaaaaaaaaaaaaaaaaaaatgcagtgggaagcaactggctaaatttactggtaaataaattaaaaaaatacatttgtgaagcactgaagccccataaattactttttaaaagtacatttatcaaacactgaagctccaatttcacacaagtaattgcatctaagatcattttatataaatgcaagagcaaaagatgaagaagatctgtgtattatctcatagtggttaaagctcatagattatagtacaaactccctgagttcatttcctggtaacataagtgacttaacctttctgtgcctcaattttctcatttataatgtggggaaaataatagcatctccctttgttgtgggaatcaaataatataagtaaagtgtgtagaacaatcgtggcatgcaagaagcactatgagtatgctgtcatcattgtgatggtctctgcattgagtatgcctgagtaagctcctatcagatctgatccttctgaagaaaataactataatctctggacaaaatacaaaaacaaaactacacccacaataataaaaccttcaattacctgaagcccctagctatagttgctctatggccgcctttacaagttaccgctaagttggtggcttaaaacaacatacatttattattttctagcgttagaggtcacaagtctaaaatcaatctccattggctaaaatcagggtgttggcagcactgcattgcttccagggactctagggaaaaatctgtttccttgccttttctagcctgcattcttggcttgtgacccatcctccaccctcaaagccagcagtgacctaccatgtcacatcgtcacaggtgccagggattaggatgtggatggttccggtgtgccattatgtgggctaccctatcatggatgtgctgagctcgagtaagatgttattgacaagatcaaatggaagtcagaacaatctagttgaaaaatgactgaaaagtctactttttgatttgacaggagcctgattttcttgcaaaccgatcaataggaatttcagtctgtcttatgtacataccaatttatagattcacatacattttgtatttaacgggcttcattctctgaaggttttctgagtttccagtagttaatcacatgatttagaatttaaatttaagtatattttcatattgcatgcatagtagacactaattacttttaacaataacattaatcaaaaataactaaaaaaatgaggatatatataaatgcaatttgttgagttcctatcgtattccagacaggcatcttcctaagccatgaatacaaaatagtgagtgtggtattatttttctttgttaatagcagcctgttcagtctttggctatagtctcttctgtgtcaggtaacagtatttgaagtatggtcacagttccaacagtagatcttcatatttcttttacatcctcattcttctggtattatggtatctgctcagaggcactacatcgtaaagccttttttgttcaagctttagggcagatagatcaaggatcaaaatgaaaagagttaatgttgtgtccacgctagttgagttttatattaatgatttctttccattaaagcacattggggtaatgtggaggaaaaggagttgatgaattttgtagttcatatgatataaattgtgttctgtatattattaaaagtaaatttctcaaatggcagggctaatgactcactggaaaaaaatgaggtttaacacttgatgttcatccattttcaaatatgctctgtatgagtctccattaagtctctgtataagtcttctgtattttaattctgtaaaatacagaattaaactccattaaaatacagaattagtatgtaaaactgtgaccatactttaaatagtgtaaatgtagagatacctgccactgaagagactatagccaaagaatgaacaggctcctattaacaaagaaaaataataccacactcatcatgttttatttgtgtttcaggaagatgcctgcttggaatatgataagaactcaacaaattgagcatttatatatatcttcatttgtttatttagttatttttaattaatgctattgttaaaagtaattagtgtctattatgcatgcaatatgcaaatatacttaaatttaaattctaaatcattgaaatgttttatttttataacagtaatatatgagtataaaataaaaatgccctcttcatagtcctcattcccccctaagataaccacagctaaaagattagtatatagccatctataactttatacataaaaatattattatataaatggaatcatatactgtcttgtcatcttttaccacctaattatatatggtttttaaaaaatcaggacacaaaatgtccagaggaagagaagattcagatttagttgatccaagctctaggtgagatttctgtagttctctttgccctgttctgtgtttgcttggttcacaggctagcttccctcatgattacaggataccttgtaggaatttcagggatgatatccaaaggcaacatcatctataggcacaaaaaggagctgactttctttatttctaaaagcaaggcactttccccaggaattccctggcagaatttccctcatacccattgtcacacacccatagctaatttatttactcgcaagagaaaatggggccagcacaactggctccgaccatggggatggcaatagggaaggatactttgaagaactaggttcttttattgggagaaggaagagaactgcatgtgaaaatgaaggtcagataatttctaacattgtctgctacatcatatagcagatacatagttagcatctgtatgctttcactggaaatgtttatatttgggaccctccatgttcttgagccaaactaggaccaacaagtaaaatctttgcctatgaaaacaaatccaactctgtgctcaggtgtctccatgaccaagaaatagaaagcgcatgtccattctccacctgtctcattcgtacagtgttcattcaacaaaaatagattgagttgcttttgtgcattctggagctagggggacaggtatgaataacaatgcctccttgctcttgagaatcttagcaatctaataataaagagagaagataaatataatgtggtatgaatctgctttcatagtaaatagttcaatcagagagcagagaagagatctaattacctacccaacactctccccttgtacgtcctcataactgtgaaatgctgaatggattagtgtgtgtttatattgtagacaatacgatgctactagctatttatatctgaaacttctattctcaaatatctagtctaacttaaccttattttaagaggctgtgttctgttacctacctattgaagaacatccttggtaaagcataaatagtacttcaataattagattataatgccaataaattcacaaatttactaattaatgtaatatttagccagcatcttgtccaaagcctctgtatataaatatttacctacctgtcatctgcaaaattgatgcagtgatgtaatggtgaatgtgattataatctcttgaagggttcatagctaaaaatattcactgagcaacaattatccatctgtcacttaggtgaatgtggaaatttgtctgtggttgaaagatgcatctttcagttttactgcctttctgtttctagaaagacaactttctactcagaagccaaggatattccatatttctaagtatttgaatgacgtggtttccagactctgtcatcaaactcaataaagacgtcaatttaagcaggatgtaatttaatcacagtggcgtagttaggcactgttttagattatgaatcttaaatgtatagattattttatttgattagtctcattcaatttggacatcttcctcagctatttgtgtgtgtttataatagaaaaaattttcatttgtaaacatatgatagcaaataaatgacatggcatatttctgattcaatcttaataaactattctgcaatttatgagtgacttaaagatatgtggaacctattgagtcagccattctcaaacatttttcttttttgagatagagtctcattctgtcacccaggctggagtgcaatggtgcaatatcctccccaaaaaacaaacacaaaagcaaaaacaacaattaacactccttcctgcatttttttggtgtgttttcttcaaagcactgaacaccttctgaagactatgtattcgcggtctgtttattgccttcctatccccattagagtgtaaatctcaggaatgcaggtctttgggtctgtggtattcgcttgctataccactaacatccaagacaatgcatgttatgtactaggtagtaaggaaatatttgctgactggagggactgcaagatggaatttatagaccaaaatcaaggggctgaaaagtaccaaaaattctgtttctgctactgtagtaacagttttgaacatatgatagagcttttttgtctgttaaatacagctgtttttgaattgcctctctattgtgggagaaaaggaatgtttttgaaaggaaaagtcagaaaataaccttttgggttcggtaatcctccatcctcatttgcctgacacagtccaggttaatacctgtggtcacagcataatcactattagttccccctttcgttgtccaaagtgtcccagtctggatgagaaattacatggttatccctagtcatggtgtctttgtgaatcagataaaacttatagaatctatttacagaaggcaatacaaataatatgcacgcatacaaaatcttgtatttcatctctgatttgaagacttaaaagaagtcttcctttttgttgagttaaataacttccttttttttttttgagttaattcttgctctgttatccatgctggagtgcagtggtatgatcatagctctctgcagcctccaatgctgggtctcaagggatcttcctgcctcctgagtagccaggaccacaggcacaagccaccatgcctggctaattaattttttttttttttttttttggtaaagataggttcttggtatattgcccaggctgtccttgaactcctgaccttaagtgatcctcccaccttaacctcccaaaatgttgggattacaggtgtgagccacctcaccccactcatacaacctctttagtgattgttgcagtcacacattctgagaagatacacttttttgcaagtaatccctgaagtgtgagagaagggcaaagaccagcaggatctacataacttgaggattacagagaccagatggaacattctgagagtgagcatgaggtcagtactcgtagtacaaactgattactttcatgtggcttctgagatcaggccacatgtgacccatgacttcctggtgaagcattaagtcttaagaccacgtggaactgtagaagggtcctttgctggaaccagaaaccttagaactgtccttagaccactgaaaaaagtacaaagagcagcgcagcaagaacacacagaatgggcctaatattgccgcgcactgatttccatagccctggaaaatgtgagccatatgtgagacaagaagaacatattgaaagccttgcagtatctgacaaaagagaaaagtaggccaggcgctgtggcccacgcctgtaatgcaagcactttgggagtctgaggtgggaggatcacttgagcccaggagtttgagaccagcttgggcaacgtggtgacactgtctctacaaaagataaaaaaaattagctggacatggtggctaatagccatagccagcatacctatagacccagctacttcagaggctgaggtggtgggattgcttgagcttgggagatcgaggctgcggtgagctgtgtttgtgccactacactccaacctgggtgacagagcgagatgctgtctcaaaaaaaaaaagcatcccaaatctcacacaacaatgaatgagattggacaatgcaaagataaagctaaatgaaagttaaataacacgagagatcacagaagaaacagcagctaaaccagcaaaattaaatacttcataagctggtgttaattgcatttgttcactctattgatatatgatgagtaaccacgttctagattctgtgtagagtaaatagtgagagcgagctgaattgctacagaggcctcttccagaataggacataaatggagcgtgtgcattgtagtgtataaacactcaataagtgattttttttttcagatgaagaaatagaaacaatgtaccaaggtcagcgattaaaaatagtgattatctttacacagtcatggactggaagttttgtttctaatagtttccagaatagaagtatttgtgtgaatgacaattatatgacatgtaaggaaattaattatatcatttattgattcttgaggtacaggctctaaagctccagcatggatttgatgtggatggtactaatgatacagatatttaagttttgttgatttgtatgtagtttccccataagaggttttttgttttttttttttttttgagacagagtttacttctcattgctcaggctggagtgctatggcatggtcttggctcactgcaacctctgcctcctggtttaaagcgattctcctgcctcagcctcccaagtagctgggattgcaggcacccaccaccacgcctggctaatttttgtgtatttttagtacagatggggtttctccatgttggccaggctggtctcgaactccttacctcaggtaatccgccggccttggcctcccaaagtgctgggattacaggcgtgagccaccacgcctggcccccataaggggtttttagttcctcgggaaactattcaagcattggttaggaagaaatatgtgcagctgggtgaatccagctaatgatcggattattctcatggcgttttgccgggggctgccttgatgatgtcttcttggatctaggtctttaacttgtctgtaactattgcacgctgtccctccagcagaccgttcctaatttaactgctatcctgtgacctcagttcttacaaaggaatctaaaactgtaacctttctttatttctggctccaattagttctttatactattcgttctctatccaaactgaactcactccctgaatatgctacacttaattcaggtatatattctccacattcttactacactaattcctcaacctagaattctgagcctttcaaaagtttaccacccctcgcttctctgcaaatgtccaaggagttgtcttccttaaacactttcatgaagctcccagccagaagtaatatttctactcgtagagttccttatcactgggtgttcacattttcttatttttgtctctcattttatatagtgattatttggtatgtattttatacattttatttacattagaatatggttttgttcaatgtccattctttaaccaacaccacattgtcttgattactagtagctttatagtaagtcttttttatttatttatttattttttgagacagagtctcgctctgtcactgggctggagtgcagtggcaccatcttggttcactgcaaccaacctctgcctcccggattcaagcagttctcctgcctcagcctcctgagtagctgggactacaggtgcgcaccaccacacccagctaatttttgtatttttagtagagacggggtttcaccttgttggccaggatggaatccatctcccgacctcgtgatccgcccacttcggactcccaaagtgctgggattacatgtgtgagccactgcgcccagcctgtagtaagttttgaagttacataatgtctgtctctgactttgttcttctccaatgttgtgattattgtgggtcttttgccttcccatataaattttaaaatcagtttgtaaaaatctacaaaataacttgctgagattttgattgggatgcactgacttgttagaatgtgtaaaaatttttctgtattcctctgtgtacctgccagtttttcatagtatatagatctaaaatgtaagactttattatatgaaatttattattcataatagatctaagcccaggaggtcaaggctgaagtgagtgctactatattccagcctgggtgaaagagagagactctgccaaaaaataaaaaataaaaaataaaaaataaaaaaaaatatatatatgtactatgtacatatcctgaggatttgaacaaattcagaaaacactgaaacagtattacatgagctggaatttatttaagagtagtaatcaacttgatttttttctattaattttagctaagcaaaagttccatcattattcatgactgatgatttctttccttatatttattgtgatgactttttaatcatacatttaaactaatacacattcctagaggtggtactctaagctgaatattttctaacaaagtctatctattattatgtctaataccacatttatcactgtattttagaaatgtttaatattaagattagtccccctcattcttttcccctgacccccaaattgttcttattcttcttgactatttatctcccaaatgaaattacttgtggtatttaatttaacttagtaattctgataagtagcctccatggaagagacttactgtattatttacttagcacccatccatccacttgaatttgttaacattaaggtttatgcaacagccactttaacgtattctgtacttagagaaaggagatattgataattttgagttagtgcatatccaggctgggtgcggtggctcatacctataatcccagtactttgggaggctgaggtgggcagatcacttgagcccaggagttcgagaccagcctgggcaacatggcgagactctgtctctacaaaaaaataaaataattagccaggtgtggtgcaacgtgcctgtagtcccagctacctggaaggctgagaggtgggaagattgcttgagcccgggaggtcaaagctaaagtgagtgctactactgccactatactccggcctgggcaaagtgagagggtctacctcaaaaaaaaaaaaaaaaagtgcatatcctgaggatttgaacaaattcagaaaacattgaaatagtattacatgaactgttcctattctaaagtaagattaatttaggaagtagtagctcttagttctttattacctagtttaaattttaaaagaaagcatttatacttaagaataaaatttctaaaccctttccttgtattgagttgtatgtgtgttagtgtatgagattgctgagtttattgtactgagtcggcttcattgtatgacagatctaaaaaaactgggagagttctgcctaatactgcaaaaccagagtaaagttaggaaattgggctaatgtttaattaagtcaatcactggccttacttttgtgcttcatttgctcaattacacttatggtaaatttctttctcgaactgatggagaaaagactttgtgatctcatgccgttaaagtacataatcccatatgatattacagtatggagagatgcttttctttttatttataattctcccacattaatgtcatgtctgtctgttctaatttagtgctttgtatttttcattttaaaagtacctggtctgctcctgtgaacatttaggtagatacctcagtttgaggtcctgtgagtctgtgattctattatttgtaaacatgtatgaaaatggaaaagaggttgggcatggtggctcacacctataatcctagcactttgggaggcaggaggattgcttgaagctgagagtttgagagcagcctgggcaacaaagagagacccctgtctctacaaaaaatttaaaaaactagctggacgtggtggtgcgcacctgtagtcccagctactcaggaggctgaggtgggaggatcactagaacctgggagttccaggctgcactgagctatgattgtaccaccctgggcaccaaagtgagaccccgtctcaaaaacaaacaaacagaaaaaacagagagggaaaaataaagaaaaatacatatgtataataataggttctcaatatatattagtttctttccctttctactttcgggatgaattcactgcttgttgaaatgtaattgactgatctaaatgctgatgttttaatgaaaagtatataatgtggatttcatttaattctgtcctaaatgacagtttgacgacatacttaacaaatccctaggcaccaaggatttggggttttgttgggaagaaaacatctaggccagaaacgatgtacttttaccttgttttgaatcttttattgtttggatcagacctcgtttaaaaatgtagaggaaaaatttaatagttccatttctacctggaaatactacctagaaatagtattagtctattagtagactaatactccagctgtgggaaaaacaaaaaacaagcaaaacacccaaacctcccccaaaccagaagttttaaaaggattaaatttaaaggatttaatcagaatatattaaatttaaaaggaaatatgattgaggcattagaaacttctgaggtagcgaggacttgaagagacctaaatcccagcagtaagagaagtgcactgaggtaggtctaacattttttataccacttttccctttggggcatttgctgagtattcatttgggcagagattaaggcagaggagcctgcaaagtttatgtaatctctggcctgacaacgtaggcagaatttgaggatacaagatcttgaaggaaaaagagaaataaggcttacactctgaagaggctttccccttgaggtatttctctgttggtggtgacgtttttattattattattacgagacagggtctcattgtccaggctggattcgaactcctgggctcaagcaatcctctctcaagtagcttggataactggcatgcaccactattccagatcatttttctgttattagcctttgggaaacaagctcagaagcttagcagaaagcagatcaaaagctcagcagtatagtaagatgtgactatcgattctatgaaacaatactaataatgtctcatgaggcctaatcttgtgctggggggccaaaaattgtatttcagtggttgctaaggaggaggggttttgataatcattccaggctctcagttggaatccttggaagttatgattactaggagagagggtgaactaaagtcttgctaagtctgaaaacctgccttcaatctactcagctcacattgaattggtgttatctgcccatcatctagctgccagccgtaggtacatcttttctggaggaagaaacagaattagcttgtatacaaattttaacacagtatctgatgtgcagtcaaacgtgttattaggcatgtaaggagtcaggatcaagagaaaaaagatctgacagtagaaatagacccataagtgatccagatattggaattatcagtcacagaccttaaaacaattggggaatctacaatgaaaaaaatcaaatggaaattcgaacactgaaaaccacagtaagtaaagtaagaactcagaagattagtttaacagcagctgggacatagttgaagaaaggattcatgaactgaaaaatagattagttgaaaatatccagactggcctatgtggagaaaagaacattaagaataaagggaaaaaatcataaaagatgcattaactgtagtaataaaatctaacatgcgagtaactggattcccaaactgtgatggatttggagctacaatagtgagattcaagattactgtcaatcctaaggaatattaatacttataaaaccacactgggttaccatagtaaaaatgctgaaaactagaaaaataaaaaatcttaaaagtaactggaggagggaacaaaatttaaaagctgtaataagactgatacctgatgacacattagaaaaagttgaatcagaagacaaaggcatgatatctttaatgttgtgaggaaaaataatcactaactctgtattatagccagataaaagtatacttcaaaagtgagagtgaagtaaagacatttccagacgcatacaaattgagataatttagatacaggagatggggttcttcaggcagaaaggaaatgataccagactgaaaagagtaaatgcaggagggaaagaagactaagtaaatatatggccaaatgtaaattaatgctaactatacaaaataatcgtattaatgccttgtggggtttaaaatatatgtgcaattaaaatgcataacgataataactcaaaaggtggaagagtataaaattgtcaaccatgttcaaagatcctttttctggaacaaggtgaagttactaatttatacttataatgaaagagttaatcacaatggaagattattaaatgtcatggatgcccatgcaacctctagggtactaaccaaaagaataagaaagaataaccaacaactttccagagggaaaaatgcaatatcaagatatttgattaatgtaaaggaaggaagaaaggagggaaaagaagtaacatatgggctgaatctgttcttaaattagattatggtgatggttcatagctgtgtaactataccaaaaagcattgaaattgtgtaccttaaataaatgaactttatgatatgtaaattatatttccataaagctatataaagagaacatgagaccaatataatacaaataatatgaaaatatgataaatataactaataaatataaactcatattttaagaataaaactaaatgttaattgactaaatacttcaatcaaaattcatggattgtcagattttattaactgcaattgtatgctgctcaccagagatatactttaagactgtcttacagaaagagtaaaagctataggatggtaaagttgtatgtgtggtaaagttgaatctaaagaaagctggtataactatgttaatatctgagaaaagggctgggcgctgtggctcatgcctgtaatcccagtactttgggaggtgagtggatcccttgaggtcaggagttcaagactagcctgaccaacgtggggaaactctgtctctgctaaaaatacaaaaaattagccaagcatggtggtgtgtgcctgcaatcccagctactcgggaggctgaggcaggagaatagcttcaatccaggaggcagaggttgcagtgagctgagatcgcaccactgtactccagcctgggtgacaaaagtaagactctgtctcaaaaaaaaaaaaaaaaaaaaaaaaaaaaaaaatatatatatatatatatatatatatatatatctctgacctctgagaaagaatttagggcagaaagtattgcgagaaatcaagatggacatttccctatgttaaaaaaagtcagtttataagaaaaataaaataattctgaatttacatgaatctaatgatatagctttcaagcttataaaaacaaaaattgacaaaattatcaggagatctcaatcaacaatcaatgtataagaccatccttctcacagtattacaataatcaggcaaataaatgaatgaggatatagaaatctgaacagtatgattaattaacatatgtattgacatttatagaacacagcaccatatgctgagaaagtacacatttgtttcaagtgcagatggagcatttaccaaaattgattatggaggaagtataagcaaatttcaaaggtttgaaataagagtgtattcctttctcatcacataattcagtaagaaataagtaacaaaaggaattgcaaatccataagcctatgaatcaacgagttaatcaaaatggcaaattataagatattttaaattggataatagtggaaaacatggatacagttaaaactgtggatacagttaaaactgattagaggaagttattcggccctaaatacataagaagaactgaataatcagttatctaaatatctcaagaagacaaaaagaaaatattaaacctggaaaatgtagaaagaagaaattatgaagtagaatttatgtagaaagcagaatttatgaagtagaaagaaatataaatcattcaagccaatagctggatttttgaaatgactaattgatgagcccttggaaagactgaatacgaaaaagagagaatataaaattaacaatattagaaatgaaaataggaaatcactgcagaacctccaaacaatgaaaagataataagagaatactgtgactagctttgttccaaagaaattaaaacattctagaaacaatatagtgttagcgaaatacagtttaccaaccctaacactagaagtgggaaatatcaactgtcttatatttattaaatgaaatgaatttataattaaagccttctcacaaagaagtccaggcccagatggcctcactggttaattccaccaaacatataaggaaacaataataccaatcttaaactctttcataatacttcccaactgattttatgaggccaacataaccctgatcctaaactatgacaaagaccatgcaaaaaaaaaaaaaaaaaaaaaaaaggtatagactaatatttatcagggactgtaggtgtagaaatcctccacaaaatattaaaatattgggaaatccatcccattgacatatcaactcaatagcctattacaaccaagttgggattcaaaaccaattattgtcacttaccttattaagataaaaaaatatttattttaacagatactataaaacatttttaaatattaaacacctattcattgtaatattagtaagtcggcatgtaagaaaagttacttaaactgatagaaattatctatcaaatgcaaaactggtgaaatattgaattctttcaaggcagtgttgaataaagttggggaaagtggatacttttggaattaaaaatgagcttggcaaagtcacagctcactataaaaattcagttatatcagttatatttttatattctagtaaaaagcaattacaaaataaaaataagtgatttcacttatattagtataatgaaacaacacatacctaggaataactctaatacaagatgtgtaaacctttacacagaaaattgagaaacattttgagaaaaataaagatgaccttaaaaaaaggataaatataccatgttcatatattgggaggctcaatattacaaagatgacagttctcttccaattgatcaggtagatcaatgcaatctcaatcaaaatcccagcaggttttcttttagtgtgcatgaaagttggaaaagccagttctaaatgaagaagagcaagattatcaaatacaactttgaagaaaagaacaaggttagagtctttgactcctggatatcaagacttattttaatgttacaataagaccgttgggtattaggacaaagacagaaaaatagagcaggatagagttcataagtagatctgtacatttgtgtcttgatgcattagtaagtgacaatgcagtgctaatggggaaaagatttattttttaataaatggtcatttggataggtatacaagggtgggtgaggagacaactcttaactccctccctaccatcttacataaaaaatcaataccaggtatgtcgcaactttcatgggaaaggcaaaataatgaactttctagaaaatttagtagctttagtagcttcgtgtcattttattttgttgttgaagatttgcgtgtgatacatttatatgtcatttgttttctagtatgtaaaatgcagataataagattacatgtaaaatgtatggctcaaagcctgccacatggtaactgctttaacttaacagaattatccatcttaaacagtaaaataaagttaatcaatttgaaagaagcaagcaaactttttttaaaaaagtttatggaatagttcatgtttatgtgccctaaatttgtaattaaaatatgaatgcacgagatgggtggatcacttgaggtcaggagttcgagatcagcctggccaacatggtgaaacaccatctctactaaaaatacaaaaattagccgggtgtggtggcgggcacctgtaatcccagctatccaggaaactgagacaggagaatcgcttgaacccaggaggcggaggttacagtgagccgagatttcaccactgcattccagcctgggtgacagagtgagactccgtctcaagaaaaaaaaaaaaaaaaaaaagaatgcaaaaatagagatgtcatttgaattatctgactttcatatagtgatataatttaaaaataattacttttatattagattcttatgtcctctctgctaaatcaagttggtgtttaaataactgttgtttcttttaagggaaattttgtttttattttgaaattttgaaatgaaattgctctttttattagtgttaatatgatagatatttaatgtaatttcctgcagatattaaaatagtcattttctttaataaaatccacatatatttttaaaattccacatacttgattcaagtttttggaaaaaatgttcctttgtcttatatgttttatgggtttgtaattgcgtaaattctaaaattggttaagtttactgtgaattttatatttccttgtcttgattagctccatttgaatcattctccttttcaccgtttgcaggaatttaaatgattcttgtcaacaagaacaagagacgtgtcatgaaatcgttctgcattgcttatgcataattacaactttattgtatgttagtcagtcttttaaccctgtggaccatttgtcttgtcttgtgtgtctagctgataattcactcccagcaatgctgttgctaagcttttctttgggtatttggatattctgtttgttacattattgctcctaattcttgcgccttgaagaactgtgtcttttgtaatatagaacattaaagagctcctgttactatcattagtaactatgactcttgattataaattctttcttctgaaaaaatcccataactttatctttgcactgatgggagtgtgtgtgtgtgtgtgtgtatgcatgtgtgaatgtctgacatgcagaagtgaacctgataatggcaaaatacaatgtctttgataatcgttgatagcactaaaaatatgcactaaatatgcactaaaatagcaccaaaaatatggcatattctattttcctgataaatattaaaaggaagtcatttaatttttttagatttttttggggaagagaataaatataaaaattcgcccttttagaggtagaaaagtgcacaattgcactgtgctggacatgccttttttgcttctgtttgccccaggatctaaaggaatggattcatcactccgataagttaatgtttaaaaggagatcttcagctttatctaacaagaaaatatcttgaaaaatgtatagtgaaaatcaggcctttctgttcagtcaagtgtttttcttaaataacaacagtgtgtttgtaaaatgcaaaggaggaagtaagtttagaacagatttttctgaaaactaagtaaattacaaaagaaggcaaaatgctctgttacaagtaacctattggatttcattagggcttcataaaattctgcctgtttatttaggctcatattttaaatatcactttataacatttttgaaataatcactctgtatgttgctttttatgcatattgcttcccaaaagcttgcttgccagagcattggagattcctagctaatagcccaagatttctgcctaagtagcattagcttcttacctaattaatgagttttctaaggaaaaaaaattacaaataaactttcatacatgaagggtgtgaagcaagccacaattcccacaggacaccaattatacattctatagatgggaggtgagttaaggaaacttacctttgagattgagtatgtctctcaatgttgaaattttttattctgatttatttgcaaggaatcctagggcgttgtagagacagtctttgttgacatatggcttttttgtttgttctgtttacagtgtgggtatgattatagtcacttaagttttaaagagccacataatgattaagagactaacattcacttataaatacaactttaaaaaataatttgattcccacataacttcagaactatttgttaaggcatgcctttatattgtgaaattgtatgaaatgctagaaagattagagtaagagaaattttactttaagtgtcatgagagaaactgtagttagacactatgtctctaaattgatctttttaattaaatcagtttttataattagaggagagtagtagttcttaaatcagaaggcctattacgtttgagaattagagaaatgttcatgtttgcttttaaataaattggattatcattattttatgtacctggtgcatttccagcctaatgtctttttgagttctgattgctaaattaactgaattctttttgtttggtgagattaaatgttgttaatgaaaaacttgaatacaattttttatgctttagaattggaagtagattgtggtgcaaggaccaaattgatatcaattcatttaaatgacagtaattgaaacattgctaggtatttgtttgtttgttatttattttggctttttctttccacagcaagttgatgtggaaaaatgggtatgtgtttccatgtatttgcaaagaaattgtgatctaaaagtgcatgcttgctctcgcctctaatctttcattttgcttcctagtgtggcttgtactattagtgttcaaactaactgcttcgttgtaatgaacacatgttttacaggtctctaatgagaaccttatgtttctggttttgcttcatctgaaatggggcatattaaagtactttccatccctagaaccacatagtttttatataatttattttgtaattattatgtgcttctggccaacagaaaggtgtttattattcatactgtctgtatatcttctgcaaaaaaaaacaaaaaacaaaaaccttgactgtttaatctcaaaattccactgcttagtccttatatttctgcatgtactgtagaagaagacaattatatatatagtcatagaaagtcatagaacctttttttttttttggtatagatggagtctcactatgttgcccaggctggtcttgaactcctggactcaagtgatccttctgcctcagcctcccaaagtgctgggattacacacaggagccactatgcccagccttatataatctgaagcagtgaaggtgaatgaccatgtagaaaaacgcagttacaggattctggaggctaattcttagagcccacattgtcacaagttgtgcaaaaagttacattgcttcttttggtctcggtttccttatctgttaggaaaaaataaaggagagaggaaatacaccaaggtaccttccaatacttaacacaccaggattctgaatggaaattaaagtctcctcaagactgatattcctactccactttctcccaagagtgcaaaacagtgaggcttttcattattgaaatgtttttgtatttgctctgtgacttatatgaagaatcctttaagattgacttaactgatggctttattttatcttcattatttttctggttttaggatccaactttcttgatgcaatattttgagaattttgccatcttttcagtgccctttcagtagtgttttccaacaatttcatgttctcttgtctttttttttaaattttttttagccctttaaagacaaggaaacttgttttttaatttaccatttctgatgacctttgaaaactatgacttgtatgttatattgctgagaataatttgaagtcttcatttctctttataaccgaactactactacttttgaccaactatttgtttttgaaatttcctaccttcctctttggctatagttaaaatgctgatccaagattcacatttttcttgtcttggtaagggaagcctgcttccttctcagaacacgtatgacacctctttgtcctgcacttccctaatatcagcatcctacaacatagccctctgtcttttcccatccttgcatcccttcacttccattacactatttctgtgccatatctggattggtattgaccaaccactcaagctgcttttacttactttgtaaatagcacttgccagtaaataaaatcaccttttcaccattgtctaaaagtcttcccttcagttcctctgtggatttggttacttttgatctgcctgtgtaatttttttctttcttattcctataataatttatttttatttgtattaagtttatttgtaaatcattgcagtagagattgccttctagttctaaggaaatatctctgatattttctgagctaaaaaccttgaggcttagtttgccaagtgactggctttataatcttatgtagcctctttattggccgtaatctctttgtctgtaggactagaatctgctttactccttttttcttaagtattactatcatgtcagtgctaaaacatattgatgccaaaatacatgttcatttggaagtcatataataaaaagattctgctgggttttagaacacggatgatcactatgtgtaaatgttgcagcaaattttagattggagctctttttttctttgaagacctatccagttcatgtcttatgctttggccattagaagtttgaaattctactgttacaggtagactttgaaggccaacagaaagtaatttaaccctctacccacctaacccaggactattgagactcctctgtggggctggtacttctctgccccttgagttaaattagaaccggtcccttgacctttctcgtattccaagagtgaaataccccacagacatttcaagtcatacagatctcatttcagtaattaaaatgcccaaatctgtataataggtgaacttgaagctaattcaagttaacttgtcaggaatcgagctggagggacggatatctgggaagctgtttcttgcttgccccgccgggttcctcacgccctacttggtacctggagagtctgccactctcaggtgctgtgggaaggagacagaagagaaagcttggcaggaaaggtctgctttgatcatagtgtcatgcactgggcttgccaggtttctaatgctgactttctctgtgggcgcttggcattggtctttcggggagcttctatccccagaaaactctgacctgagcttctcttgctgcagacaaatgcgtgtccactagctggtcagtggcaattccttactcttcctgggagttcttggtggcctctgagacaatcccaagctggatctgtctagtatggcccccatttgcttgttggcagtgtggtcacttcccagttgcagccctgtaggactacacccagccttttgcctctcaatctccaggcatagtaagacaccagtctacagtgtcccagctcccagggatatcaggtatccctgaagctacccttgctaggcttgaagtgagggcaagcttgggaggcaggcaggattccaggcccacctgcacttctctccaagaaaccatttctttgctgcctggcctctaataatacactgggggtgggcagtcagctaagggcagccagtggatttgcagccaccctctttgacatccctgcttaggtggcataacaatatgttttgggacgtgatagtacaggtgctcctccagttagggtgaggttatatcctgaaaagcccttcagaagttgaaaatatcgtaagccaaaaatgcatgtaatacacctaacctacagaacattatagcttagcttagcctcccttaaacgggctgagaacacttacattagcttacagtggggcaacatcatctaacacaaagcctattttatactaaagtgttgaatatctcatgtaatttattgaatactatactgaaagtaaaaaatacaatgattgtatgggtaatcaaaatacagttcctgctgaatgcaaatcactttcacattgtcgtacagttgaaaaatcataaatagaaccattgttaagtcagggaccttcttgtaaatccccttcggagactggtcattgttttgttttgggacagtgacctgttttaacttctgttatacttgcttgtctagggtgcgtttaccaccaccagattaagacctttgggagccttacaaacttaaatatatatgaaaattaaaatggcttctgtataaatcttctgataacattattctggatagtttcatcaaaggtccatcaatcactttctctttctttctcctttctccctcccctctccatccccttttccctaattcccttcctgccttcatctccttccttctctccatcatcactgtctctttgagcagctgtgcatctgtttaatcacaagcccactgagttatcaaatgctgctctttacactttaaaagagaataaaactcctgagtactggataagaaagtaactaatggtcatttccatctactccactcctagcccttttctaacctataccattggtttccaatgtggaggcctaggaatcaggtcatgagtggggtgaagaaagcaaaaggatgaaatagactgtgagattgttgtctatgaagcattgtctatagactgagtgatgtcttacacatgtattttctcttaaaaaatatgatgatgctgttgtaattgttaaaatccataatcatttcaaatgtttctacctggagagttgtttaaaaatgcattttaacacacgactgctattatgtgagaacctgcgaaatgctttcaagctatcaaggttcattcatgtttgaaatggtttttaaaaatatatggagtcttctgtattctgacatgggcaattttttatgtgtaggtgatgatctgaatagagggcaggctttgtatgtcttcaagacatcagtatccatttgccctctgtagaaatcaatgtgttgcttttgatttagagaaattctctatcgagacaagctgggaaatgaagaaagccctagatacctcttctatttcatgttttaggcatttatgcagctctttgctttataaacctgtcacagagataaagtgtgagaacttatagtagcttatcggaatcatttatttttctttacccttctaaagaagaactacataattgtatgtacatatatatgtcaaatttattatactcgaaagagaagacattccatttaactactgtctgtacagagctgtttataggttgaaaaagaacttgctacaaatctgccgaaaaatagaatgaattctttttaaacatatctgcatctcacataactagattccagtgatggtcattaatatgagtgacacaagggcatttcaatagaatgtattattatgtgtataattgctcattcatggactggtggacaagcctctttcagatgatttctgtacctgtaatcaatatgtctgtcttttcagagcatcttggacatttttagcaggggaatcctggaaagtgaagcatttccataggatttatatagatattttttaagacagggtcttgctctgtcacccaagctggagcgtggctcaagtgatagtcccacctcagcctcctgagtagctgggactacaggcatgtgccaccatgcctggctaactttttaattttttgtagagacagtgtctcactatggtgcccaagctagtcttgcactcctgggctcaagcagatactcctgccctggcctcccaaagtgctgggattacaggcatgagccactgcacccagacacatattttcatcataagagcgatagttgaagcttttcaacctcatatgctattaggtagtatatattgatatttggaactcagaattgttgttaatttttttattctaaaaattattgggactatcattaattaactatagctacctagaaatcctagtctttggtggtgaaatcactttgttttctcaacataatgaagtttacctgatatttcctcacaaaggagaggttaccggttggagacttatggagaatcctagcatcttgatacgtggagaggaaaagatggcgaaaaggcttaaatttgatgtatattttgtgtacagagtagggtattttgcctccttaatggaaacttcacgctagaatgacttctgaggctattctgaaattgttgcatttgataatcctttttaatggcaagcctccaaatgtatggtgctttgtcactaaatccttatctatataatttttgaggagacatttcaataacagtttcttttaaaagttctaaggtaggtttttagaagcgttcctttttaaaaatcagtttttagctcatgaaactgaaataccagttttaaaaagtcaaggttgaactgaaaggcatcattataatagaggagctttgctacattacctgaattattgtttccttctttgcaccaagaacagattaaatatgcattctcttgatatccagtatttgttagcaattttcagagtaaagtattgctatttgatttgtttttctattcataatcatttgcatttgctaagagagtgccttaggtcttttagatctactacactgagtggctcatgcttacagacgaaagatgattctgcttctttttgtcatccatttgacacccagggccgtctatgtgttaaaatgtagcaatagcaatttctcttcctggcttccatgttaatgtggcatgtgcccccaggttttgcttgtatttggagctccatctgatgtgtcaattccggattaatctaaacctgggacactgagttactgacatctgcagttttagcctaaagaactaaaatttccacctttgtgtttctatgcaaattaatcatcatccaaaaaattctaagacagcacgttggaatccatgctgcgtgcctgtcctataattgcccatatagtcatcccttactctattttgtgtaatgaaattttagctcttgtctattgaacttctcattagtgaaataaatcaaatagttaatttatttgctaaacttattgtaagcacaaaaacgacattctccacacccaaagaggctgctttcttcaatatttatctttatgtgtttcttttttctactttccatcatatcaatttttaaagggtttttgttgttattctttttctgtttgttgcttttatcatacagaagcagtgcttacattgaattaaactaatatactcatcttttttgttttaatgcgctttgctttcttcttttcccttaaacgatccctttgaatgtctcctgatgctataattttggtaatatctagcacttaaaccaatttgaacaagaaatggtttgtacagaaagttattgtcaataatgccactaataccacaaatctttcatgttcttgattatcttttgaaagtgatgtttgtgtcttacatctgctcttctttctcattctgcagaaggatatttcagcatttgctatttttcagtattcattctattttgtcgtgtatgcagattgctctcctctgtgtattattcttcctacagtcagacatgacttagcaatgccgaatagcagacctcggtcacttggctgaccactcttttttttgttaatacctactacatttttaaagtggttcaacttagatacctgacaaaatcaggctgagaaaattgtctgaaggaaaatctcccaaacctagcttaattaaaaaaaaaaaaatctcatgaagaaagtatctcttcacaaaccatgtgctgtaccaaaatgttaggtttttgtgtttctttgaattaaggcttaataatgattttgcctaattctttagtgatatctctaccattagtgagtcacatgtgattggtcagtttaacatggaaagtaagtttcctgatattatgtatgattaatatcagttataaaaaggtaactaaacagcctgtattttctaagccaaggatgaaagcttctttacaatttttccttgaaattgtaatattgaaaatgaaaaaattagtctcaacagaatttccctttaataatatacatattactatgtaaatatgtttcagggaaaaattgtaatatttacaaatatatatattggaaaacttttccttgctgtttttcttacaactagtgtctctatggtatcttttctccataggacttagacatcaacctgacaacaaattcagccctaagtttggaaatatatcattgttttctttttcatactctttcatctgtgtaactctcctaggaataagtagtcaaagaaactttagaggtgtaaagaaaaatgaagttatttgtaaatttgcatatgcacacacatacgtacacattatgttttgtagaagaaaaaagagctattatataatttatgaaaggagactgtgtcattttaagagaaaaagaaatgtttttgtgatttttctgtgaaattcacctttcacaatattcagtgtctaagaaattgcttactgttattttgcatgaaacgagagccgctcattgcagcatattagtttacgatctaccttacctgttttaatctactgtttttattaaaatgagttgggttgctgtttgtttttatcactgcatttttatgcaaaagatgcaagttggttttttaaaaagcatttgcagaggatcaatttttaagatgataccttttttggtgcttaaataggttgtcaatggaatcactgaaagtagagcagcatgttttcacagaaaaaggaagaaactaacgtcaaaacatttttaaagagttttgcttgctacggcttttctctagcatgtagttgatactaagttttgttgcgataacatggtgaatgtttttttttatgtttattgtttacatagctccatttaggataacggagtctttataaataagagatatttttaaggaagtctttgagatcgtgtctatttaatgtttcctctcttttccttatgcccctacag

SEQ ID NO: 20 - DNA, 21 bp, Homo sapiens
  source 1..21: mol_type = genomic DNA
caagttgatgtggaaaaatgg

SEQ ID NO: 21 - DNA, 513 bp, Mus musculus
  source 1..513: mol_type = genomic DNA
ggctgtcttctggacctccttcctgattagcagaataccggtggggctgcttcattaactgagattgcttttgtgcactgaggtgaactgctggtaatgaaaagttggaacactgtcgtgtgttctttaaagggaagtggttttcgggtctgggggtcacctttagttgacttaggcagccatcggaaacattgctaggaactttgttttgttttgttttttaaaatttttttcggctttttcttttcacagcaggtggatgtggaaaagtgggtatgtgttcccctgcatttacaaaggacttttgtggctgaaaagcgcatgctcgcagctgcctctgagctttttaattttgcttcctagcatagcttcctttagtgtttgaactaactgctcggctacaatgaatacatttcacaggtcaccactcagcaccttttgtgtctctgaattatcatcaccatcaacaggctccttcaaaggactctccactcgaaaaccagattgttttta

SEQ ID NO: 22 - DNA, 21 bp, Mus musculus
  source 1..21: mol_type = genomic DNA
caggtggatgtggaaaagtgg

SEQ ID NO: 23 - protein, 51 aa, Mus musculus
PDLSICTFVLEQSLSVRALQEMLANTVEKSEGQVDVEKWKFMMKTAQGGGH

SEQ ID NO: 24 - protein, 51 aa, Rattus norvegicus; identical to SEQ ID NO: 23

SEQ ID NO: 25 - protein, 51 aa, Macaca mulatta; identical to SEQ ID NO: 23

SEQ ID NO: 26 - protein, 51 aa, Oryctolagus cuniculus; identical to SEQ ID NO: 23

SEQ ID NO: 27 - protein, 51 aa, Pan troglodytes; identical to SEQ ID NO: 23

SEQ ID NO: 28 - protein, 51 aa, Sus scrofa
PDLSICTFVLEQSXSVRALQEMLANTVEKSEGKVDVEKWKFMMKTAQGGGH

SEQ ID NO: 29 - protein, 51 aa, Canis lupus; identical to SEQ ID NO: 23

SEQ ID NO: 30 - protein, 51 aa, Homo sapiens; identical to SEQ ID NO: 23

SEQ ID NO: 31 - protein, 51 aa, Ovis aries
PDLSICTFVLEQSLSVRALQEMLANTVEKSEGKVDVEKWKFMMKTAQGGGH

SEQ ID NO: 32 - protein, 51 aa, Bos taurus; identical to SEQ ID NO: 31

SEQ ID NO: 33 - protein, 44 aa, Equus caballus
PDLSICTFVLEQSLSVRALQEMLANTVEKSEGKFMMKTAQGGSH

SEQ ID NO: 34 - DNA, 20 bp, synthetic construct
  source 1..20: mol_type = other DNA
ccggacctgtctatctgcac

SEQ ID NO: 35 - DNA, 20 bp, synthetic construct
  source 1..20: mol_type = other DNA
ctgtaggaatggcgtagcaa

SEQ ID NO: 36 - DNA, 20 bp, synthetic construct
  source 1..20: mol_type = other DNA
gacctgtccatctgcacctt

SEQ ID NO: 37 - DNA, 21 bp, synthetic construct
  source 1..21: mol_type = other DNA
accactgtaggaatggcgtag

SEQ ID NO: 38 - DNA, 20 bp, synthetic construct
  source 1..20: mol_type = other DNA
ccagacctctccatctgcac

SEQ ID NO: 39 - DNA, 20 bp, synthetic construct
  source 1..20: mol_type = other DNA
ataggaatggcgcagcaata

SEQ ID NO: 40 - DNA, 20 bp, synthetic construct
  source 1..20: mol_type = other DNA
caggacagaagacccctcag

SEQ ID NO: 41 - DNA, 20 bp, synthetic construct
  source 1..20: mol_type = other DNA
ggccacaacagctctttttc

SEQ ID NO: 42 - DNA, 20 bp, synthetic construct
  source 1..20: mol_type = other DNA
caggtggcagatggctctat

SEQ ID NO: 43 - DNA, 20 bp, synthetic construct
  source 1..20: mol_type = other DNA
gattgtacaaaggggccatc

SEQ ID NO: 44 - DNA, 21 bp, synthetic construct
  source 1..21: mol_type = other DNA
gatggcaaatggctctttaca

SEQ ID NO: 45 - DNA, 20 bp, synthetic construct
  source 1..20: mol_type = other DNA
ccttttcctctgcttggaca

SEQ ID NO: 46 - DNA, 25 bp, synthetic construct
  source 1..25: mol_type = other DNA
cctcttacctcagttacaatttata

(19) United States
(12) Patent Application Publication
(10) Pub. No.: US 2026/0159843 A1
(43) Pub. Date: Jun. 11, 2026

(54) MICROEXON TARGETING FOR MODULATION OF RYR2 CHANNEL ACTIVITY

(71) Applicant: UNIVERSITY OF VIRGINIA PATENT FOUNDATION, Charlottesville, VA (US)

(72) Inventors: Neslihan Muge Martinez, Charlottesville, VA (US); Md Imrul Hasan Chowdhury, Earlysville, VA (US)

(21) Appl. No.: 19/356,782

(22) Filed: Oct. 13, 2025

Related U.S. Application Data
(60) Provisional application No. 63/730,678, filed on Dec. 11, 2024.

(51) Int. Cl. C12N 15/113; A61K 31/712; A61K 31/7125; A61P 9/06; C12N 15/86

(52) U.S. Cl. CPC C12N 15/1138; A61K 31/712; A61K 31/7125; A61P 9/06; C12N 15/86; C12N 2310/11; C12N 2310/315; C12N 2310/321; C12N 2310/322; C12N 2310/3233; C12N 2320/33; C12N 2750/14143

(57) ABSTRACT

The present disclosure provides antisense splice switching oligonucleotides (SSOs) that cause RyR2 microexon 4 skipping, and methods of use thereof. The antisense SSOs modulate activity of the RyR2 calcium channel, and are useful for treatment of disorders characterized by an overactive RyR2 channel. The disclosure also provides AAV vectors and compositions comprising said antisense SSOs.

### CROSS-REFERENCE TO RELATED APPLICATIONS

This application claims the benefit of and priority to U.S. Provisional Application No. 63/730,678 filed on Dec. 11, 2024. The content of which is incorporated by reference in its entirety.

### STATEMENT REGARDING FEDERALLY SPONSORED RESEARCH

This invention was made with government support under HL157780 awarded by the National Institutes of Health. The government has certain rights in the invention.

### REFERENCE TO AN ELECTRONIC SEQUENCE LISTING

The content of the electronic sequence listing (18250600057.xml; Size: 75,512 bytes; and Date of Creation: Oct. 13, 2025) is herein incorporated by reference in its entirety.

### BACKGROUND

Ryanodine receptor 2 (RYR2) tightly regulates calcium release from sarcoplasmic reticulum and maintains intracellular calcium (Ca²⁺) homeostasis in the heart. Mutations or dysregulation of RYR2 leads to various cardiac abnormalities including catecholaminergic polymorphic ventricular tachycardia (CPVT), idiopathic ventricular fibrillation, atrial fibrillation, heart failure, and cardiomyopathies. Most RYR2 mutations are gain of function mutations that result in excessive calcium leak, in turn tachycardia and increasing the chance of sudden death. Therefore, RYR2 is a major target for treatment of many heart conditions. So far, drugs that inhibit RYR2-mediated calcium leaks have not been very effective in treating all these heart conditions. There is still a need for more specific RYR2 modulators.

### SUMMARY

In an aspect, provided herein is a composition comprising at least one antisense splice switching oligonucleotide (SSO), the at least one antisense SSO comprising a sequence selected from SEQ ID NOs: 1, 4, 15, or 16, or a sequence having at least 90% identity thereto.

The at least one antisense SSO may consist of a sequence selected from SEQ ID NOs: 1, 4, 15, or 16, or a sequence having at least 90% identity thereto. The at least one antisense SSO may be a morpholino. The at least one antisense SSO may comprise a phosphorothioate backbone. The at least one antisense SSO may comprise at least one modified nucleotide selected from a 2′-O-methyl modified ribose (2′-OMe), a 2′-O-methoxy ethyl modified ribose (2′-MOE), a locked nucleic acid (LNA), and a lipid modified nucleotide.

In another aspect, provided herein is an adenosine-associated virus (AAV) vector comprising an antisense SSO comprising a sequence selected from SEQ ID NOs: 1, 4, 15, or 16, or a sequence having at least 90% identity thereto. The antisense SSO may consist of a sequence selected from SEQ ID NOs: 1, 4, 15, or 16, or a sequence having at least 90% identity thereto. The vector may be an AAV9 vector.

In another aspect, provided herein is a pharmaceutical composition comprising the antisense SSO described herein, and a pharmaceutically acceptable carrier.

In another aspect, provided herein is a method for modulating ryanodine receptor 2 (RyR2) channel activity in a subject in need thereof, the method comprising administering to the subject a pharmaceutical composition comprising an agent that reduces expression of Ryr2 microexon 4. The agent may comprise at least one antisense splice switching oligonucleotide (SSO) comprising a sequence selected from SEQ ID NOs: 1, 4, 15, or 16, or a sequence having at least 90% identity thereto. The at least one antisense SSO may comprise a sequence selected from SEQ ID NOs: 4, 15, or 16, or a sequence having at least 90% identity thereto. The at least one antisense SSO may consist of a sequence selected from SEQ ID NOs: 4, 15, or 16, or a sequence having at least 90% identity thereto. The at least one antisense SSO may be a morpholino. The at least one antisense SSO may comprise a phosphorothioate backbone. The at least one antisense SSO may comprise at least one modified nucleotide selected from a 2′-O-methyl modified ribose (2′-OMe), a 2′-O-methoxy ethyl modified ribose (2′-MOE), a locked nucleic acid (LNA), and a lipid modified nucleotide. The at least one antisense SSO may be packaged in an AAV vector. The AAV vector may be an AAV9 vector.

The subject may have a disorder selected from a heart condition. The heart condition may be selected form an arrythmia, heart failure, diabetic heart disease, atrial fibrillation, catecholaminergic polymorphic ventricular tachycardia, arrhythmogenic right ventricular dysplasia type 2, and a cardiomyopathy.

### BRIEF DESCRIPTION OF THE DRAWINGS

The patent or patent application file contains at least one drawing in color. Copies of this patent or patent application publication with color drawings will be provided by the Office upon request and payment of the necessary fee.

The present disclosure will be better understood and features, aspects, and advantages other than those set forth above will become apparent when consideration is given to the following detailed description thereof. Such detailed description makes reference to the following drawings.

FIGS. 1A-1D. Evolutionary conservation and strategical placement of RYR2 microexon 4 encoded amino acids located in the dimer interface region of the channel pore. A) Conservation analysis of RyR2 amino acids coded by microexon 4 and microexon 5 among eleven different mammals with their respective average heart rates. In descending order, the sequences are SEQ ID NOs: 22-33. B) Tetrameric structure of the open RyR2 calcium channel reveal that microexon 4 encoded amino acids are within the N-terminal domain located near the channel pore. C) RyR2 microexon 4 encoded amino acid residues are located near the dense columns forming the dimer interface region of the channel pore. D) Zoomed image marking the location of microexon 4 encoded amino acids in RyR2 open channel structure.

FIGS. 2A-2B. Ryr2 microexon 4 inclusion is regulated during mouse heart development coinciding with T-tubule formation. A) Ryr2 microexon 4 regulation during mouse heart development. Representative gel images and quantification of Ryr2 microexon 4 and exon 75 (control) inclusion at different stages of heart development: embryonic day 18 (E18), Day 5 (D5), Day 8 (D8), Day 10 (D10) and adult (98 day old). Data are mean±standard error mean (n=3 from three independent experiments). ****P=<0.0001; ***P=0.0002; **P=0.0025; by one way ANOVA followed by a Tukey's multiple comparisons test. B) Representative images of T-tubules formation throughout different developmental stages of mouse heart development: Day 5 (D5), Day 8 (D8), Day 15 (D15), Day 20 (D20).

FIGS. 3A-3E. Ryr2 microexon 4 inclusion is regulated by RBFOX2 in embryonic mouse hearts and in primary cardiomyocytes. A) Aberrant Ryr2 microexon 4 splicing in Rbfox2 mutant embryos. RBFOX2 and α-smooth muscle actin (alpha-SMA) immunostaining in E9.5 Control (Rbfox2^{flox/flow}) vs Rbfox2-CKO (Rbfox2^{flox/flow}; Nkx2-5^{Cre/+}) mouse hearts reveal efficient deletion of RBFOX2 in the cardiac region of the embryos. Nuclei were stained with DAPI. Scale bar=100 μm. B) Representative gel images and quantification of Ryr2 microexon 4 inclusion in control versus Rbfox2-CKO mouse hearts determined by RT-PCR. Data are mean±standard error mean (n=3 heart per genotype). **P=0.0048; by analysis of unpaired Student's t-test. C) Representative gel images and quantification of Ryr2 microexon 4 inclusion in rat cardiomyocytes treated with non-targeting siRNA or two different RBFOX2 targeting siRNAs determined by RT-PCR. Data are mean±standard error mean (n=3 from three independent experiments). ****P=<0.0001; **P=0.0047; by one way ANOVA followed by a Tukey's multiple comparisons test. D) Schematics of neonatal mouse cardiomyocyte isolation and experimental design. E) Representative gel images and quantification of Ryr2 microexon 4 inclusion in mouse cardiomyocytes treated with scrambled or RBFOX2 targeting siRNA determined by RT-PCR. Data are mean±standard error mean (n=6 from three independent experiments). ****P=<0.0001; by unpaired Student's t-test. FIG. D was made using Biorender.

FIGS. 4A-4D. RNA binding proteins RBFOX2 and QKI regulate AS of RYR2 microexon 4. A) RBFOX2 binding consensus site is present in intron 4 of RYR2 pre-mRNA flanking microexon 4. B) Representative gel images and quantification of RYR2 microexon 4 inclusion in HEK293 stable cells expressing wild type version (RBFOX2^{WT}) or an RNA binding mutant of RBFOX2 (RBFOX2^{RRM}). Data are mean±standard error mean (n=4 from four independent experiments). ****P=<0.0001; by one way ANOVA followed by a Tukey's multiple comparisons test. C) Representative gel images and quantification of RYR2 microexon 75 inclusion in HEK293 stable cells expressing RBFOX2^{WT} or an RNA binding mutant of RBFOX2 (RBFOX2^{RRM}). D) Representative gel images and quantification of Ryr2 microexon 4 and exon 75 inclusion in mouse cardiomyocytes treated with scrambled or QKI targeting siRNA determined by RT-PCR. Data are mean±standard error mean (n=4 from two independent experiments). ***P=<0.001, ****P=<0.0001; by unpaired Student's t-test.

FIGS. 5A-5G. Microexon 4 skipping via SSO decreases RyR2-mediated Ca² transients and synchronous beating rate of cardiomyocytes. A) Representative gel images and quantification of Ryr2 microexon 4 inclusion in primary mouse cardiomyocytes treated with control (control SSO) or Ryr2 microexon 4 targeting splice switching antisense oligonucleotide (E4-SSO). Data are mean±standard error mean (n=7 from three independent experiments). ****P=0.0001; by unpaired Student's t-test. Quantification of B) cardiomyocyte beating, C) active sites/cell, D) Peak Amplitude, E) Rise time 10-90%, F) Duration at ½ amplitude, and G) decay time at half amplitude (t½) in primary mouse cardiomyocytes treated with control or Ryr2 microexon targeting E4-SSO. Data are mean±standard error mean (n=7 from three independent experiments). *P=0.0213; *P=0.0235; *P=0.0406; **P=0.0049; ***P=0.0005; *P=0.0223; **P=0.0014; **P=0.0046; **P=0.0076; *P=0.0146 by one way ANOVA followed by a Tukey's multiple comparisons test.

FIGS. 6A-6H. Ryr2 microexon 4 skipping adversely affects caffeine-induced channel activity in mouse cardiomyocytes. A) Representative gel images and quantification of Ryr2 microexon 4 inclusion after E4-SSO treatment. Data are mean±standard error mean (n=4 from three independent experiments). ****P=0.0001; by unpaired Student's t-test. B) Representative illustrations of basal and caffeine-induced calcium pulse C) Ca²⁺ signal frequency before and after caffeine treatment in control and E4-SSO treated cardiomyocytes. Data are mean±standard error mean (n=4 from three independent experiments). ***P=0.0001; by unpaired Student's t-test. D) Quantification of Ca²⁺ concentration in control and E4-SSO treated cardiomyocytes after caffeine induction. Data are mean±standard error mean (n=4 from three independent experiments). ***P=0.0001 unpaired Student's t-test. E) Representative gel images and quantification of RyR2 microexon 4 inclusion after E4-SSO treatment. Data are mean±standard error mean (n=4 from three independent experiments). ****P=0.0001; by unpaired Student's t-test. F) Representative traces calcium transients before and after caffeine induction in Ryanodine-treated cardiomyocytes (control vs E4-SSO). G) Ca²⁺ signal frequency after caffeine treatment in cardiomyocytes (control vs E4-SSO). H) Measurement of calcium concentration after caffeine induction in Ryanodine treated cardiomyocytes (control vs E4-SSO).

FIGS. 7A-7D. L-type Ca²⁺ channel induced RyR2 Ca²⁺ sparks in mouse cardiomyocytes are impaired upon RyR2 antisense oligonucleotide treated (E4-SSO) treatment. A) Pseudocolor images represent Ca²⁺ sparks (green-color events) in a single frame in mouse cardiomyocytes from control and E4-SSO groups in the absence or presence of L-type Ca²⁺ channel activator Bay K8644 (10 μM). B) Representative images show automatically detected Ca²⁺ sparks in the absence or presence of Bay K8644, within the recording duration of 30 s. Each + sign indicates one Ca²⁺ spark event. C) Ca²⁺ Sparks activity (Events/μm²/minute) after Bay K8644 (10 μM) treatment in the absence or presence of ryanodine (5 μM) in cardiomyocytes (control vs E4-SSO) (*P<0.05 vs Basal, #P<0.05 vs Bay K8644 treatment; repeated 1-way analysis of variance). D) Ca²⁺ Sparks amplitude after Bay K8644 (10 μM) treatment in the absence or presence of ryanodine (5 μM) in cardiomyocytes (control vs E4-SSO) (*P<0.05 vs Basal, ###P<0.001 vs Bay K8644 treatment; repeated 1-way analysis of variance).

FIGS. 8A-8D. Identification of SSOs that modulate human RyR2 microexon 4 inclusion. SSOs that target splice sites or RNA binding protein RBFOX2 and QKI binding sites were transfected to HEK293 Flip in cells expressing RBFOX2^{WT}. A) Representative gel images of human RYR2 microexon 4 and exon 75 inclusion in Flip-in HEK293 stable cells inducibly (doxycycline) expressing Flag-Rbfox2 (WT) treated with control (control SSO-10 μM) or 8 different hRYR2 targeting SSOs (10 μM) after Doxycycline induction. Quantification of B) human RYR2 microexon 4 inclusion, C) human RYR2 exon 75 inclusion and D) RBFOX2 mRNA levels in Flip-in HEK293 cells inducibly expressing Flag-Rbfox2 (WT) treated with control (control SSO-10 μM) or hRYR2-SSOs (10 μM) after doxycycline induction. Data are mean±standard deviation mean (n=2 from two independent experiments). ****P=<0.0001; by one way ANOVA.

FIGS. 9A-9B. Ryr2 microexon 4 is included in brain cortex and cerebellum at later developmental stages. A) Representative gel images and quantification of Ryr2 transcripts containing or lacking microexon 4 and exon 75 in the brain cortex at the same developmental stages. Data are mean±standard error mean (n=3 from three independent experiments). ****P=0.0001; by one way ANOVA followed by a Tukey's multiple comparisons test B) Ryr2 microexon 4 and exon 75 splicing at different stages during cerebellum development in the brain. Data are mean±standard error mean (n=3 from three independent experiments). ****P=0.0001; by one way ANOVA followed by a Tukey's multiple comparisons test.

FIG. 10. Similar levels of RBFOX2^{WT} AND RBFOX2^{RRM} expression in HEK293 stable cells. Representative gel images and quantification of RBFOX2 mRNA levels in Flp in HEK293 stable cells expressing WT or RNA binding mutant of RBFOX2 after doxycycline induction. Data are mean±standard error mean (n=4 from four independent experiments). ***P=0.0007; ***P=0.0008; by one way ANOVA followed by a Tukey's multiple comparisons test.

### DETAILED DESCRIPTION

The inventors demonstrate herein the role of RyR2 microexon 4, which encodes seven amino acids located at the dimer interface region of the RyR2 channel pore, in regulating the RyR2 channel's calcium release activity. This work has implications to ultimately control the heart rate in patients having an overactive RyR2 channel.

In a first aspect, provided herein is a composition comprising at least one antisense splice switching oligonucleotide (SSO), the at least one antisense SSO comprising or consisting of a sequence selected from SEQ ID NOs: 1-18, or a sequence having at least 90% identity thereto. The at least one antisense SSO may comprise or consist of a sequence selected from SEQ ID NOs: 1, 4, 15, or 16, or a sequence having at least 90% identity thereto. The composition may comprise one antisense SSO or several different antisense SSOs. The composition may comprise between one and 18 SSOs, or any number or range in between.

Ryanodine receptor 2 (RyR2) is a receptor encoded by the Ryr2 gene and found primarily in cardiac muscle. RyR2 is a major component of a calcium channel located in the sarcoplasmic reticulum that supplies ions to the cardiac muscle during systole. The RyR2 channel is composed of RyR2 homotetramers and FK506-binding proteins found in a 1:4 stoichiometric ratio.

An antisense splice switching oligonucleotide is a single-stranded synthetic oligonucleotide designed to bind specific pre-mRNA sequences and sterically prevent RNA-binding proteins or spliceosome components from interacting with their target transcript.

The terms “oligonucleotide”, “polynucleotide”, and “nucleic acid” are used to refer to DNA or RNA molecules, or fragments thereof. These terms include double- and single-stranded DNA, as well as double- and single-stranded RNA. A “polynucleotide” or “oligonucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. Oligonucleotides are typically shorter in length (e.g. 2-100 nucleotides), while polynucleotides are longer chains of nucleotides (e.g. longer than 100 nucleotides). For use in the present compositions and methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

In embodiments, the antisense SSOs described herein are modified. These are phosphorodiamidate morpholino oligomers (PMOs) that are uncharged, nuclease-resistant and about ˜<25-base pair long oligos. They block RNA by base pairing and steric hindrance. Different version of antisense oligos and morpholino oligos can be used to cause RyR2 exon 4 skipping. By way of example, and not limitation, these modifications may increase stability of the SSO, alter the pharmacokinetics or therapeutic index of the SSO or decrease off-target effects of the SSO. Typical modifications include those to the phosphate backbone and ribose modifications. For example, modifications to the type of nucleotide linkage or backbone include phosphorothioate (PS) backbone. Other backbone modifications may include a stereodefined backbone configuration or mesylphosphoramidate (MsPA) linkages. The antisense SSO may comprise one of these backbone modifications, or a combination of two or more within the same oligo. Additional modifications may include nucleotide modifications. Nucleotide modifications may include, but are not limited to, 2′-O-methyl modified ribose (2′-OMe), 2′-O-m ethoxy ethyl modified ribose (2′-MOE), 2′fluoro (2′-F), Locked nucleic acid (LNA), Constrained ethyl (cEt), Tricyclo-DNA (tcDNA), Peptide nucleic acid (PNA), 5-methyl-cytosine (m⁵C), and N-acetylgalactosamine (GalNAc) modifications. The antisense SSOs can also be formulated as morpholino oligonucleotides. In such embodiments, the riboside moiety of each subunit of an oligonucleotide of the oligonucleotide reagent is converted to a morpholine moiety. Morpholinos may also be modified, e.g. as a peptide conjugated morpholino, a phosphorodiamidate morpholino (PMO), etc.

Additional modifications known in the art include 5′ and 3′ modifications. Typical 5′ modifications may include, without limitation, inverted deoxythymidine bases, addition of a linker sequence such as C6, addition of a cholesterol, addition of a reactive linker sequence which could be conjugated to another moiety such as a PEG. Typical 3′ modifications may include, without limitation, inverted deoxythymidine bases, and inverted abasic residues. Additional modifications may include those which allow for localization, for example, targeting the antisense SSO to the heart, or enhancing cellular distribution or cellular uptake of the SSO. Such modifications include chemically linking one or more moieties to the SSOs. Such moieties include, but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

Multiple modifications may be used in one SSO and individual nucleotides may be modified differently from other nucleotides in the SSO. Also multiple SSOs can be combined together.

“Percentage of sequence identity”, “percent similarity”, or “percent identity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantial similarity” of polynucleotide or peptide sequences means that a polynucleotide or peptide comprises a sequence that has at least 75% sequence identity. Alternatively, percent identity can be any integer from 75% to 100%. Embodiments described herein have at least: 75%, 80%, 85%, 86%, 87%, 88%, 89%, 90%, 91%, 92%, 93%, 94%, 95%, 96%, 97%, 98% or 99% identity compared to a reference sequence using the programs described herein; preferably BLAST using standard parameters, as described.

The antisense SSOs may be made by any techniques known in the art, such as, for example, solid phase synthesis. One method for synthesizing oligonucleotides on a modified solid support is described in U.S. Pat. No. 4,458,066. The antisense SSOs are synthesized in vitro and do not include antisense compositions of biological origin. The molecules of the invention may also be mixed, encapsulated, conjugated or otherwise associated with other molecules, molecule structures or mixtures of compounds, as for example, liposomes, nanoparticles, receptor targeted molecules, or other delivery vehicles for assisting in uptake, distribution and/or absorption.

Any of the antisense SSOs described herein may be coupled to a small nuclear RNA (snRNA) molecule, such as U7. U7 snRNA is an RNA molecule required for histone pre-mRNA processing. An anti-histone portion of the U7 snRNA may be exchanged with the antisense RyR2 SSO. The RyR2 specific SSO sequences can be inserted into the U7 scaffold for splicing modulation of microexon 4. U7-SSO can also be delivered as plasmid or by adeno-associated viruses.

Provided herein are constructs comprising a nucleotide sequence encoding any of the antisense SSOs described herein coupled to a promoter. The term “construct” refers to a recombinant polynucleotide, i.e., a polynucleotide that was formed artificially by combining at least two polynucleotide components from different sources (natural or synthetic). For example, the construct may comprise the nucleotide encoding the antisense RyR2 SSO operably linked to a promoter that (1) is associated with another gene found within the same genome, (2) from the genome of a different species, or (3) is synthetic. As used herein, the term “promoter” refers to a DNA sequence that regulates the transcription of a polynucleotide. Typically, a promoter is a regulatory region that is capable of binding RNA polymerase and initiating transcription of a downstream sequence. Promoters may be derived in their entirety from a native gene, may be composed of elements derived from multiple regulatory sequences found in nature, or may comprise synthetic DNA segments. A promoter is “operably linked” to a polynucleotide if the promoter is connected to the polynucleotide such that it may affect transcription of the polynucleotide. Constructs can be generated using conventional recombinant DNA methods. In embodiments, the promoter is a U7 promoter. Alternatively, U1 or U6 snRNA promoters can be used.

Any of the antisense SSOs or constructs described herein may be packaged in an adenosine-associated virus (AAV) vector. Adeno associated viruses (AAVs) are non-pathogenic viruses that belong to the genus Dependoparvovirus. AAV are small, nonenveloped viruses that have a linear single-stranded DNA genome that is approximately 4.7 kilobases (kb) in size. AAV viruses are replication defective, meaning that the production of AAV virus requires coinfection with helper virus(es). As used herein, the term “vector” refers to a virus particle that is used to deliver genetic material into cells. The term includes the vector as a nucleic acid genome structure packaged in a capsid for administration, as well as the vector genome after introduction into the nucleus of a host cell into which it has been introduced. AAVs offer several advantages for use as gene therapy vectors: AAV-based gene therapy vectors cause a very mild immune response, can infect both dividing and quiescent cells, and persist in an extrachromosomal state without integrating into the genome of the host cell. At least 11 AAV serotypes have been identified, the serotypes differing in their tropism, or the types of cells they infect. AAV serotype 9 (AAV9) has tropism for cardiomyocytes and has a relatively long plasma half-life compared to other AAV serotypes. In embodiments, the AAV vector is an AAV9 vector. However, other AAV capsid serotypes (e.g. AAV8, AAV6, AAV1, AAVrh.74, AAVMYO, etc) could also be used, including hybrid capsids and capsids that have been artificially engineered to target specific cell types.

In a third aspect, provided herein is a kit comprising at least two antisense SSOs comprising sequences selected from the group comprising or consisting of SEQ ID NOs: 1-18, wherein each antisense SSO is in a separate container. The at least two antisense SSOs may comprise sequences selected from the group comprising or consisting of SEQ ID NOs: 1, 4, 15, or 16.

In a fourth aspect, provided herein is a pharmaceutical composition comprising any of the antisense SSOs described herein, and a pharmaceutically acceptable carrier, excipient, or diluent. The carrier, excipient or diluent depends upon the desired use for the composition and may range from being suitable or acceptable for veterinary uses to being suitable or acceptable for human use. The composition may optionally include one or more additional compounds.

When used to treat or prevent a disease or symptoms of a disease, such as arrythmia, heart failure, diabetic heart disease, atrial fibrillation, catecholaminergic polymorphic ventricular tachycardia, arrhythmogenic right ventricular dysplasia type 2, or a cardiomyopathy, the compositions described herein may be administered singly, as mixtures of one or more compounds or in mixture or combination with other agents (e.g., therapeutic agents) useful for treating such diseases and/or the symptoms associated with such diseases. Such agents may include, but are not limited to, blood thinners, ACE inhibitors, beta blockers, calcium channel blockers, anticoagulants, etc. The compounds may be administered in the form of compounds, or as pharmaceutical compositions comprising a compound.

Pharmaceutical compositions comprising the compound(s) may be manufactured by means of conventional mixing, dissolving, granulating, dragee-making, levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. In some embodiments, the antisense SSO may be lyophilized. Antisense SSO may be reconstituted in sterile water.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fdlers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, fdms or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, di chlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt.

Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration. The compositions described herein will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of composition administered will depend upon a variety of factors, including, for example, the particular indication being treated, the mode of administration, whether the desired benefit is prophylactic or therapeutic, the severity of the indication being treated and the age and weight of the patient, the bioavailability of the particular composition, the conversion rate and efficiency of delivery under the selected route of administration, etc. In some embodiments, modification to the compositions described herein may alter the bioavailability or therapeutic index. For example, some compositions may be administered daily, weekly, monthly or every 2, 3, 4, 5, or 6 months, or yearly.

Determination of an effective dosage for a particular use and mode of administration is well within the capabilities of those skilled in the art. Effective dosages may be estimated initially from in vitro activity and metabolism assays. For example, an initial dosage for use in animals may be formulated to achieve a circulating blood or serum concentration of the composition that is at or above an IC₅₀ of the particular composition as measured in an in vitro assay. Calculating dosages to achieve such circulating blood or serum concentrations taking into account the bioavailability of the particular composition via the desired route of administration is well within the capabilities of skilled artisans. Initial dosages can also be estimated from in vivo data, such as animal models. Animal models useful for testing the efficacy of the active metabolites to treat or prevent the various diseases described above are well-known in the art. Animal models suitable for testing the bioavailability and/or metabolism of compositions are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active composition, the bioavailability of the composition, its metabolism kinetics and other pharmacokinetic properties, the mode of administration and various other factors, discussed above. Dosage amount and interval may be adjusted individually to provide plasma levels which are sufficient to maintain therapeutic or prophylactic effect. For example, the compositions may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compositions may not be related to plasma concentration. Skilled artisans will be able to optimize effective dosages without undue experimentation.

In a fifth aspect, provided herein is a method for modulating ryanodine receptor 2 (RyR2) channel activity in a subject in need thereof, the method comprising administering to the subject a pharmaceutical composition comprising an agent that reduces expression of Ryr2 microexon 4. “Modulating” the RyR2 channel refers to reducing activity of the channel, and thereby decreasing calcium release through the channel. In embodiments, the RyR2 channel overactivity is reduced by between about 30 and about 50% In embodiments, the RyR2 overactivity channel is reduced to by about the same activity shown in a subject having normal expression levels of Ryr2 microexon 4.

Agents that reduce expression of Ryr2 microexon 4 include, but are not limited to, any of the antisense SSOs, AAV vectors, or pharmaceutical compositions described herein; a CRISPR/Cas-based system (e.g. Cas9, deactivated Cas9, Cas12a, or Cas13 base editors) engineered to disrupt splice sites, splice enhancers, or intronic regulatory elements to achieve RyR2 microexon 4 skipping; and a small molecules and/or peptides that bind to microexon 4.

CRISPR/Cas-based systems may include guide RNAs designed to target splice donor or acceptor sites of RyR2 microexon 4; Cas nucleases fused to splicing-modulating domains (dCas9-SR proteins); and base editors or primer editors to disrupt splice sites and regulatory motifs. Small molecules include, but are not limited to, those that bind near microexon 4 encoded amino acids, and reduce RyR2 calcium release activity. Peptides include, but are not limited to, RNA binding protein decoys engineered to sequester splicing enhancers, and small peptides that bind to microexon 4 encoded amino acids of human RyR2.

A “subject in need thereof” refers to having or at risk of having a disorder characterized by overactive RyR2 channel activity, including but not limited to, arrythmia, heart failure, diabetic heart disease, atrial fibrillation, catecholaminergic polymorphic ventricular tachycardia, arrhythmogenic right ventricular dysplasia type 2, and a cardiomyopathy.

As used herein, the term “administering”, refers to dispensing, delivering, or applying the therapeutic agent, to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, “preventative,” “preventing,” “prevent” and the like refer to partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

### Introduction

Cardiac contraction is dependent on excitation-contraction (EC) coupling in the myocardium (1). EC coupling requires effective communication between L-type Ca²⁺ channels (LTCCs) on transverse-tubules (T-tubules) of the sarcolemma and Ryanodine receptor type-2 (RyR2) on the sarcoplasmic reticulum (SR) membrane. Depolarization of the plasma membrane activates voltage-gated L-type Ca²⁺ channels causing Ca²⁺ influx and triggers RyR2-mediated Ca²⁺ release from SR known as calcium-induced Ca²⁺ release (CICR) (1). CCIR mediates cardiac muscle contraction during each heartbeat.

RYR2 mutations cause a wide range of heart diseases including catecholaminergic polymorphic ventricular tachycardia, arrhythmogenic right ventricular dysplasia type 2, RyR2 Ca²⁺ release deficiency syndrome as well as cardiomyopathies (2-15). Dysregulation of RyR2 is also implicated in heart failure, diabetic heart disease and atrial fibrillation (16-21). RyR2 gain of function mutations cause leaky channel activity leading to tachycardia and possibly sudden death (3,4). Understanding the complex regulation of RyR2 channel is essential and will lead to new lines of therapeutics that modulate RyR2 Ca²⁺ channel activity and treat many heart diseases.

Due to RyR2's vital function in the heart, it is regulated transcriptionally and post-translationally as well as via interactions with proteins, ions and molecules (20,27-29). However, post-transcriptional regulation of RYR2 is currently poorly understood. Microexons of 3-30 bp length (30) have recently gained attention because of their essential roles in nervous system development and high conservation (30-33). Importantly, aberrant microexon splicing in specific genes is linked to cognitive impairment and autism like diseases (32). Microexon inclusion is facilitated by RNA binding proteins (34-36). RNA binding protein families including RBFOX and PTBP are implicated in microexon regulation in the brain (31,37). These RNA binding proteins are also expressed in the heart. However, very little is known about microexon regulation by RNA binding proteins in the heart (37,38).

RYR2 mRNA is composed of ˜105 exons and harbors two uncharacterized microexons that are unusually small (microexon 4: 21 bp and microexon 5: 15 bp). RYR2 microexons encode for short amino acid sequences in the N-terminus of the protein (1-906 aa), which is essential for channel pore opening and closing (39). Microexons 4 and 5 encoded amino acids are thought to have protective roles in restoring RyR2 protein structure when exon 3 of RyR2 is deleted in patients (40). Despite the critical location of RYR2 microexon encoded aminoacids, it is unclear how they contribute to RyR2 channel function due to their small size in the enormous RyR2 channel.

In this study, we investigated the contribution of RYR2 microexons to RyR2 channel function at RNA level and their regulation by RNA binding proteins during heart development. We found that RYR2 mRNA isoforms including microexon 4 was predominant in adult hearts, while Ryr2 mRNA isoforms excluding microexon 4 were favored in embryonic hearts. Notably, alternative splicing of Ryr2 microexon 4 was tightly regulated during heart development coincident with the timing of T-tubule formation and maturation. We identified RNA binding proteins RBFOX2 and Quaking (QKI) asregulators of RYR2 microexon 4 in cardiomyocytes. We showed that Ryr2 microexon 4 splicing is altered in Rbfox2 mutant embryos with congenital congestive heart failure. We successfully generated an antisense splice switching oligonucleotide (SSO) that specifically causes Ryr2 microexon 4 skipping, diminishing RyR2 Ca⁺² channel activity and L-type calcium channel activated calcium sparks without affecting Ryr2 mRNA levels. Our findings have implications for controlling RyR2 channel activity in human heart diseases with nucleotide level precision using SSO.

### Materials & Methods

### Animal Studies

All the animal studies were carried out in compliance with the NIH Guidelines and approved by the Institutional Animal Care and Use Committee of UTMB and UVA. We purchased both Rbfox2^{flox/flox} mice (Stock no: 014090) and C57BL/6J mice (stock no: 000664) from Jackson labs, whereas Nkx2.5^{Cre/+} mouse was a kind gift from Dr Robert Schwartz's lab. These Nkx2.5^{Cre/+} mice became an effective tool to study Nkx2-5 mediated cardiac development via cre-loxp system (41). To obtain the Rbfox2− Het CKO (Rbfox2^{flox/+}, Nkx2.5^{Cre/+}) mice, Rbfox2^{flox/flox} mice were initially crossed with Nkx2.5^{Cre/+} mice. Rbfox2-CKO (Rbfox2^{flox/flow}, Nkx2.5^{Cre/+}) embryos were finally generated by mating Rbfox2− Het CKO (Rbfox2^{flox/+}, Nkx2.5^{Cre/+}) males with Rbfox2^{flox/flow} females. For time mating, E0.5 was designated as the noon of the day of the plugs. Pregnant uterus obtained from time mating was kept in cold PBS (phosphate-buffered saline) after dissection until the harvesting of embryos and their hearts. To determine the genotype of the embryos, tail or yolk sac genomic DNA was used. Mice heart, cortex and cerebellum were collected at different developmental stages (E18, NB, D5, D8, D10, P14 weeks) for alternative splicing check of RyR2-exon 4 and exon 75.

### Processing of Embryos and Paraffin Sectioning

Embryos processing and paraffin section were performed according to the previously described protocol⁴². Control (Rbfox2flox/flox) and Rbfox2-CKO (Rbfox2flox/flox, Nkx2.5^{Cre/+}) E9.5 embryos were fixed in 4% paraformaldehyde buffered with 0.1 M sodium phosphate of pH 7.2 at 4-C for 24 hours and washed with 1×PBS (#46-013-CM, Corning) immediately. Then embryos were dehydrated using sequential concentrations of ethanol (70-100%) (#E7023, Sigma). Xylene (#534056, Sigma) was used to clear the embryos before embedding into paraffin blocks with desired orientation. Embryos were sectioned 7 μm thick using a microtome (Microtome, HM2035) and were placed on glass slides.

### Immunofluorescence Staining and Imaging

Paraffin sections were incubated for 12-14 hours at 56° C. followed by xylene treatment for 20 minutes to deparaffinize and dehydrate tissues sections. Decreasing concentrations (100-50%) of ethanol were then used to wash the slides for 5 min at each concentration. The sections were treated with sodium citrate buffer (10 mM, pH 6.0) for 20 minutes in a steam chamber to expose antigens. The sections were then blocked in 3% BSA in PBST (0.2% Triton X-100) at RT for 1 hour followed by incubation with the following primary antibodies: Rbfox2 (A300-864A, Bethyl laboratories) and alpha-smooth muscle actin (NB300-978, Novus biologicals) in a humidifying chamber for 14-16 h at RT. Then, slides were washed with PBS containing 0.1% Triton X-100 before incubation with secondary antibodies for 2 hours at 37° C. Slides were then washed four times with PBS containing 0.1% Triton X-100 followed by incubation with 4′,6-diamino-2-phenylindole dihydrochloride (DAPI, #MP01306, Invitrogen) stain in the dark for 30 min at RT. Excess stain was removed by washing the slides with PBS. Slides were mounted onto the coverslips using Mowiol mounting media and sealed using nail polish. A confocal laser-scanning microscope (LSM 880META, Carl Zeiss) at the University of Texas Medical Branch, Optical Microscopy Core facility, was used to get fluorescence images.

### Modeling RYR2 Protein Structure

The model structure of RyR2 was created using atomic coordinate from 6J18 (43) using UCSF chimera. The amino acids around microexon 4 were highlighted in red to show the location of microexon 4 encoded amino acids within the structure.

### RNA Extraction and RT-PCR for Alternative Splicing Analysis

RNA was extracted from cells and mouse heart tissues using TRIzol (#15596018, Invitrogen) using company's established protocol. RNA concentrations were measured using RNA concentrations were measured using SYNERGY LX Microplate Spectrophotometer (BioTek) prior to cDNA construction. Alternative splicing of exons was evaluated by performing semi-quantitative RT-PCR according to the previously described protocol (44,45). Primers were designed to assess AS of microexon 4 and 5 or exon 75 of RyR2.

### Primer Sequences for Mouse, Rat and Human Microexon 4 and 5 Alternative Splicing:

mRYR2e4F:
(SEQ ID NO: 34)
CCGGACCTGTCTATCTGCAC
mRYR2e4R:
(SEQ ID NO: 35)
CTGTAGGAATGGCGTAGCAA
rRYR2e4F:
(SEQ ID NO: 36)
GACCTGTCCATCTGCACCTT
rRYR2e4R:
(SEQ ID NO: 37)
ACCACTGTAGGAATGGCGTAG
hRYR2e4F:
(SEQ ID NO: 38)
CCAGACCTCTCCATCTGCAC
hRYR2e4R:
(SEQ ID NO: 39)
ATAGGAATGGCGCAGCAATA

### Primer Sequences to Detect Mouse, Rat and Human Exon 75 AS:

mRYR2e75F:
(SEQ ID NO: 40)
CAGGACAGAAGACCCCTCAG
mRYR2e75R:
(SEQ ID NO: 41)
GGCCACAACAGCTCTTTTTC
rRYR2e75F:
(SEQ ID NO: 42)
CAGGTGGCAGATGGCTCTAT
rRYR2e75R:
(SEQ ID NO: 43)
GATTGTACAAAGGGGCCATC
hRYR2e75F:
(SEQ ID NO: 44)
GATGGCAAATGGCTCTTTACA
hRYR2e75R:
(SEQ ID NO: 45)
CCTTTTCCTCTGCTTGGACA

PCR was performed using 5 μl cDNA, 25 M dNTPs and 100 ng primer pair (forward and reverse) with Biolase Taq polymerase (Bioline BIO-21042) in a 20 μl reaction. PCR amplification condition for each reaction was 95° C. 45 s; 60° C. 45 s; 72° C. 1 min for 32 cycles. PCR products were run on 5% non-denaturing polyacrylamide gels followed by staining with ethidium bromide. The DNA bands were imaged using a Bio-Rad XR Imaging system and quantified using Image Lab software and sequenced after gel extraction followed by PCR and Sanger Sequencing. At least three independent experiments were carried out for quantification and statistical analysis using GraphPad Prism.

### T-Tubule Imaging

Hearts were excised and rinsed in KB solution (composition: 90 mmol/L KCl, 30 mmol/L K2HPO4, 5 mmol/L MgSO4, 5 mmol/L pyruvic acid, 5 mmol/L β-hydroxybutyric acid, 5 mmol/L creatine, 20 mmol/L taurine, 10 mmol/L glucose, 0.5 mmol/L EGTA, 5 mmol/L HEPES, pH 7.2). The heart was then cannulated via the aorta and connected to a Langendorff apparatus, where it was initially perfused with calcium-free Tyrode solution for 3 to 5 minutes at 37° C. This was followed by perfusion with calcium-free Tyrode containing Liberase TH (Roche Applied Science) at different concentrations (20 μg/ml for P15-P20 hearts and 15 μg/ml for P5-P8 hearts) for 10 to 15 minutes at 37° C. Following digestion, the heart was rinsed with 3 ml of KB solution to remove residual collagenase. The hearts were then minced, gently agitated in KB solution, and filtered through a 210 m polyethylene mesh to obtain ventricular myocytes. These ventricular cardiomyocytes at different timepoints were stained with 5 mol/L Di-8-ANEPPS (ThermoFisher, D3167) for 10 minutes in normal Tyrode solution containing 1.8 mmol/L Ca²⁺ to stain the T-tubules. The stained cells were imaged using a confocal microscope (LSM 510, Carl Zeiss) equipped with a 40× oil immersion objective.

### Isolation of Mouse Cardiomyocytes

Mouse cardiomyocytes were isolated from newborn mouse heart ventricles using commercially available primary cardiomyocyte isolation kit (#88281Y, Thermo Fisher Scientific) according to the manufacturer's protocol. Briefly, freshly dissected neonatal hearts were minced into 1-3 mm³ pieces followed by washing with 500 μL ice cold HBSS twice for the removal of blood. After addition of 200 μl of reconstituted Cardiomyocyte Isolation Enzyme 1 (with papain) and 10 μL of Cardiomyocyte Isolation Enzyme 2 (with thermolysin) to each tube, minced hearts were then incubated at 37° C. for 30-35 minutes. After removal of the enzyme solution, each tube was washed twice with 500 μL ice cold HBSS. The tissue was broken down by pipetting up and down after adding 500 μL of complete DMEM to allow cell isolation. Cells were then grown into gelatin coated plates after determination of cell concentration and cell viability.

### Mouse Cardiomyocyte Transfections

A monolayer of day 1 mouse cardiomyocytes was plated in a 35 mm glass bottom culture dish (Part No: P35GCOL-0-10-C, MatTek Corporation) precoated with a gelatin substrate at a density of 3×10⁶ cells/well and maintained in complete Dulbecco's modified Eagle's medium (DMEM-#88287, Thermo Fisher Scientific) for Primary Cell Isolation medium containing 10% fetal bovine serum (FBS) and 1% penicillin-streptomycin. Cells were then transfected with: a) scrambled siRNA (Invitrogen AM4611) or RBFOX2 siRNA (Invitrogen siRNA ID #s96620) using Lipofectamine RNAiMAX (#13778150, Thermo Fisher Scientific) and b) Control-SSO (Standard Control, Gene Tools LLC, seq—‘CCTCTTACCTCAGTTACAATTTATA’) (SEQ ID NO: 46) (15 M) or RyR2 microexon4-SSO (Gene Tools LLC, seq-ACCTGCTGTGAAAAGAAAAAGCCGA) (SEQ ID NO: 1) (15 μM) using endoporter (Gene Tools LLC) for 48 hours. SSO was designed to block 5′ splice site of microexon 4. Transfected cells were then imaged and harvested for RNA extraction.

### Rat Cardiomyocyte Transfection

Rat cardiomyocytes were purchased from Cell Application Inc. (R357-25) and were maintained in rat cardiomyocyte growth media (Cell Application Inc., R313-500) at 37° C. in a 5% CO₂ humidified incubator. Media was replaced at every 2 days and cells were treated with trypsin (0.05% trypsin/EDTA, Invitrogen 25300-054) before plating. Rat cardiomyocytes were then plated on gelatin (Sigma G1890)-coated T25 flasks (Nunc 12-565-351) at a density of 2×10⁵ cells. Beating cardiomyocytes after 7-10 days after plating were transfected with Accell Non-targeting siRNA #1 (D-001910-01-20, Dharmacon), Rbfox2-si1: Modified siRNA (CTM-194373, Dharmacon) and Rbfox2-si2: Modified siRNA (CTM-194377, Dharmacon) using Accell siRNA Delivery Media (B-005000-100, Dharmacon).

### RBFOX2 Induction in Flp-in HEK293 Stable Cells

Expression vector pcDNA5/FRT/TO was used to clone Human FLAG-tagged RBFOX2^{WT} and RBFOX2^{RRM} mutant based on previously described protocol (42). Co-transfection of 4 g pOG44 recombinase and 0.4 g RBFOX2^{WT} or 0.4 g RBFOX2^{RRM} plasmid in Flp-in T-REx 293 cells were performed using lipofectamine 2000 (#11668019, Invitrogen) after plating them on six well plates. 100 μg/ml hygromycin was then used to select for cells expressing the plasmids. RBFOX2 expression was induced by incubating the cells with 1.0 μg/ml of doxycycline for 24 hours.

### Neonatal Cardiomyocytes Ca²⁺ Imaging

Cultured mouse cardiomyocytes were incubated with Fluo-4 AM (2.5 μM, ThermoFisher #F14201) and pluronic acid (0.004%) at 37° C. for 10 minutes. Ca²⁺ images were acquired at 30 frames per second using an Andor Revolution WD (with Borealis) spinning-disk confocal imaging system (Andor Technology) comprising an upright Nikon microscope with a 60× water-dipping objective (numerical aperture, 1) and an electron-multiplying CCD camera. Fluo-4 was excited using a 488 nm solid-state laser, and emitted fluorescence was captured using a 525/36 nm band-pass filter. Ryanodine (RyR2 inhibitor; 5 μM-5 mins), Caffeine (2 mM, RyR2 agonist) and L-type Ca²⁺ channel activator Bay K8644 (10 μM) were used to study the function of RyR2 (46). Cells were imaged immediately after.

Ca²⁺ images were analyzed using custom-designed SparkAn software (source code available at https://github.com/vesselman/SparkAn). Regions of interest of 1.7 μm² (5×5 pixels) were placed at the peak event amplitudes for each event site to obtain fractional fluorescent traces (F/F₀) and the properties of RyR2 Ca²⁺ signals were analyzed. For each event site, frequency, amplitude, rise time (time of rise from 10% of maximal amplitude to 90% of maximal amplitude), duration (maximum width at half-maximal amplitude), and decay half-life (t_{1/2}, time of decay from peak to half-maximal amplitude) were determined.

Ca²⁺ concentration in neonatal cardiomyocytes was estimated using the maximal fluorescence method. Fractional fluorescence traces (F/F₀) were obtained using polygon drawing on each cell as a region of interest (ROI), excluding the nucleus.

Estimates of [Ca²⁺], were made using the Fmax equation (47):

$${Ca}^{2 +} = Kd \frac{F / Fmax - 1 / Rf}{1 - F / Fmax}$$

where F is fluorescence measured within an ROI, Fmax is the fluorescence intensity of Fluo-4 at a saturating maximum Ca²⁺ concentration, K_{d} is the dissociation constant of Fluo-4 (340 nM), and R_{f} (=100) is this indicator's ratio of maximum to minimum fluorescence measured in vitro at saturating and zero Ca²⁺ concentrations. Fmax was obtained individually for each culture dish by adding the Ca²⁺ ionophore, ionomycin (10 μM), and 20 mM external Ca²⁺ at the end of the experiment.

### Statistical Analysis

GraphPad Prism software was used to conduct all the statistical analysis. Significance between two groups was measured by Unpaired t-test, whereas significance among multiple groups was conducted by one-way analyses of variance (ANOVAs) followed by a Tukey's multiple comparisons test. All the statistical details were given in each figure legend.

### Human SSO Sequences:

hRYR2-E4SSO-01:
(SEQ ID NO: 4)
ATACATGGAAACACATACCCATTTT
Intron 3a:
(SEQ ID NO: 2)
ATATCAATTTGGTCCTTGCACCACA
Intron 3b:
(SEQ ID NO: 3)
ACCTAGCAATGTTTCAATTACTGTC
Intron 4a:
(SEQ ID NO: 14)
ACTTTTAGATCACAATTTCTTTGCA
Intron 4b:
(SEQ ID NO: 15)
GATTAGAGGCGAGAGCAAGCATGCA
Intron 4c:
(SEQ ID NO: 16)
ATAGTACAAGCCACACTAGGAAGCA
Intron 4d:
(SEQ ID NO: 17)
TGTTCATTACAACGAAGCAGTTAGT
Intron 4e:
(SEQ ID NO: 18)
ATTGTCTTCTTCTACAGTACATGCA

### Results

### Evolutionary Conservation and Strategic Location of RYR2 Microexon 4 Encoded Amino Acids

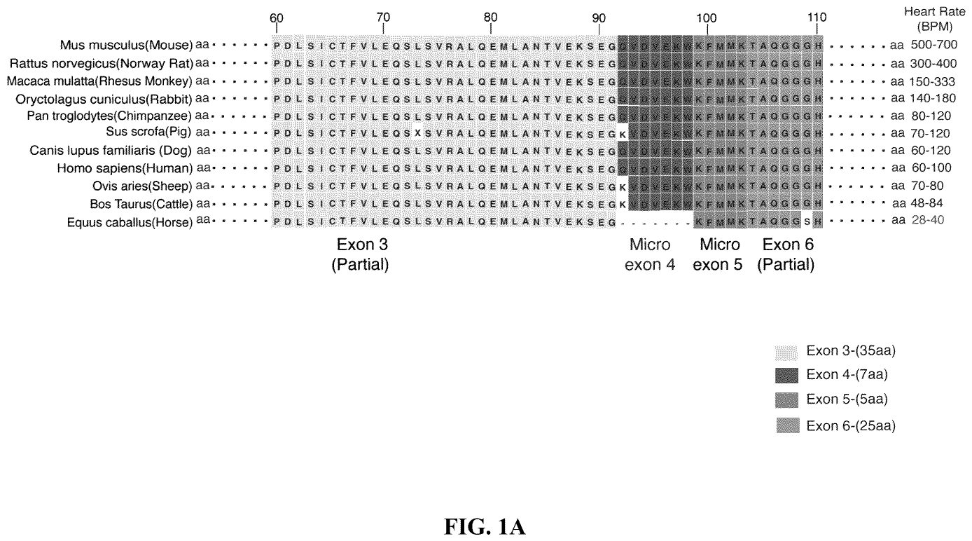

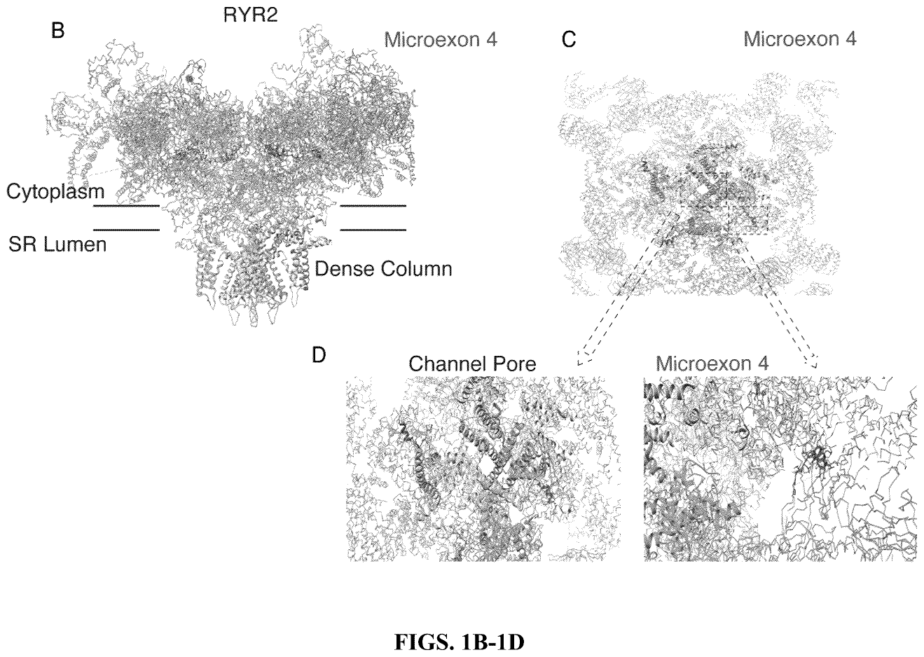

Microexons are highly conserved. To determine whether RYR2 microexons 4 and 5 are also conserved, we aligned amino acid sequences encoded by RYR2 microexons 4 and 5 among eleven different mammals using Jalview (48). RYR2 microexon 5 encoded five amino acids (99aa-103aa: K-F-M-M-K) are highly evolutionarily conserved among these mammals (FIG. 1A). To our surprise, microexon 4 encoded seven amino acids (92aa-98aa: Q/K-V-D-V-E-K-W) are not present in horse RyR2 protein (FIG. 1A). Horses have very low heart rate (28 to 40 bpm) in comparison to these other mammals (FIG. 1A) (49). This evolutionary analysis suggests that lack of microexon 4 may correlate with lower heart rate.

RyR2 forms a homotetramer structure and that inter- and intra-subunit interactions especially in the N-terminus are critical for RyR2 channel function (50-53). Microexon 4 encoded amino acid residues are located in the N-terminal region of the RyR2 (40,54) but it is not clear where in the known RyR2 channel structure.

We utilized atomic coordinate from 6JI8 to generate the RyR2 tetramer model in an open channel confirmation with the amino acids around microexon 4 (FIG. 1B, in red) (43). Cryo-electron microscopy mapping of this open channel structure revealed that microexon 4 encoded residues are located very close to the dense “columns” projecting toward the channel pore (54) (FIGS. 1C and 1D). These results indicate that RYR2 microexon 4 coded amino acids are in a critical region near the RyR2 channel pore and may contribute to Ca²⁺ release activity of RyR2.

### Ryr2 Microexon 4 Inclusion Follows T-Tubule Formation During Heart Development.

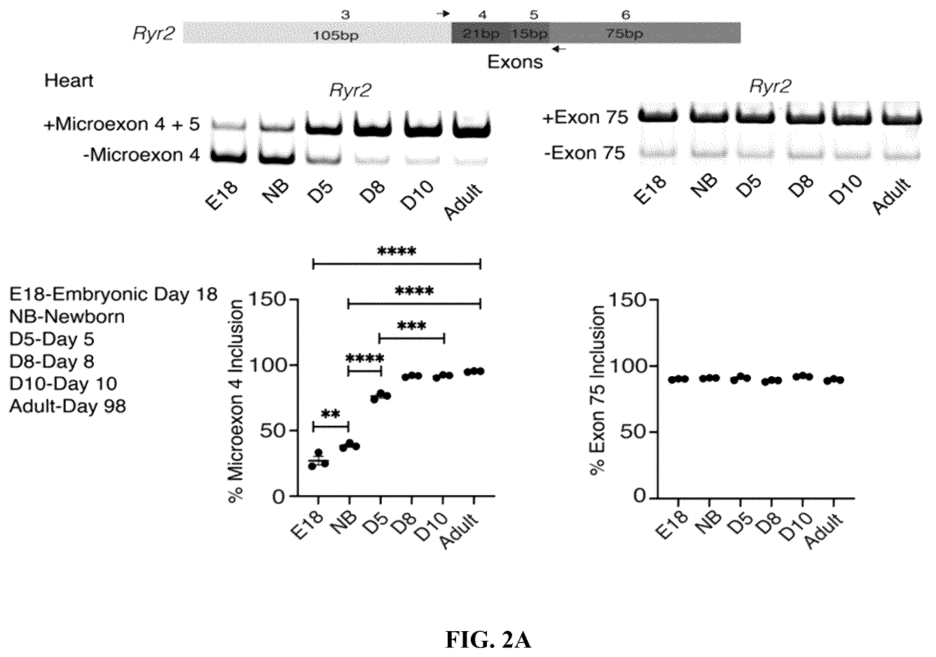

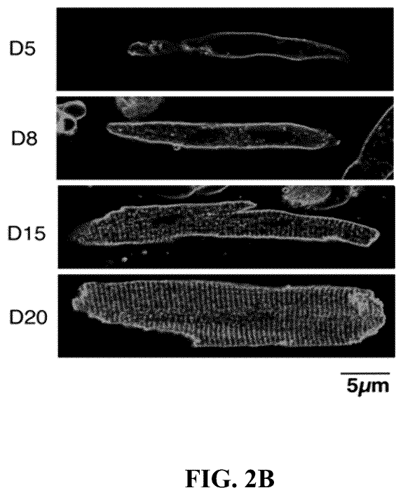

If Ryr2 microexon 4 is an indicator of RyR2 channel activity, we reasoned that this microexon might be regulated during heart development. RyR2 and T-tubules have an intimate relationship to support CCIR in the heart. T-tubules form and mature after birth between D5 and D10 in mice hearts (24,25). Ryr2 has a modest role in Ca²⁺ signaling during EC-coupling in embryonic cardiomyocytes in comparison to adult cardiomyocytes (26). To test whether Ryr2 microexon 4 is developmentally regulated, we isolated heart tissues from mice at different developmental stages: Embryonic day 18 (E18), Newborn (NB), early postnatal (Day 5-D5, Day 8-D8, Day 10-D10) and adult (98 days-old or 14 weeks). We designed primers on exon 3 and exon 6 to check inclusion of both microexons 4 and 5 by RT-PCR and sequenced the amplified DNA bands. Sequencing analysis of the included and excluded DNA bands confirmed that microexon 4 but not 5 was alternatively spliced (FIG. 2A). Strikingly, Ryr2 microexon 4 inclusion started to increase after birth around day 5 and was almost completely included at day 8 and in adult stages in mouse hearts (FIG. 2A, left graph). Importantly, the dramatic change in Ryr2 microexon 4 inclusion followed T-tubule formation and maturation at postnatal stages (FIGS. 2A vs 2B) (25). As a control, we examined alternative splicing (AS) of Ryr2 Exon 75, which is also a small exon (36 bp). Exon 75 inclusion was unaffected during heart development (FIG. 2A, right graph). Ryr2 transcripts included exon 75 throughout heart development and thus was used as a control for Ryr2 mRNA levels for the rest of the studies.

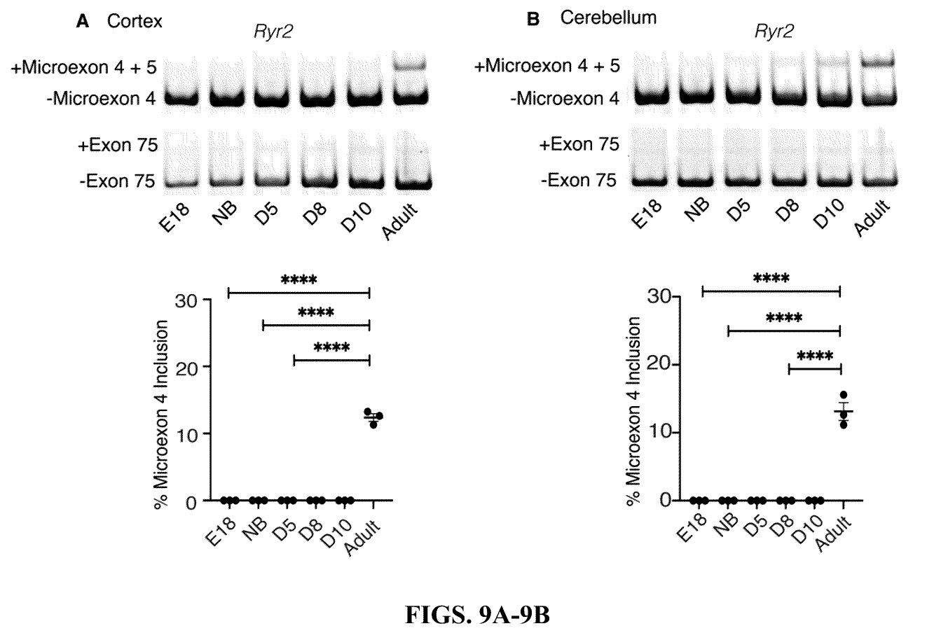

It has also been noted that RYR2 is expressed in the cerebellum and cerebral cortex of the brain (55-58). Thus, we checked whether microexon 4 regulation is also conserved during brain development. In both cerebral cortex and cerebellum, Ryr2 microexon 4 was completely excluded in Ryr2 transcripts throughout embryonic and early postnatal stages but was more included in adult (98 day-old) mice brain (FIG. 9). Ryr2 exon 75 splicing did not change throughout development stages in both cerebral cortex and cerebellum during brain development and served as a control for Ryr2 levels.

These results indicate that Ryr2 microexon 4 is tightly regulated via AS during mouse heart development correlating with the formation of mature T-tubules in the developing heart. The increased inclusion of microexon 4 was conserved in both heart and brain tissues, suggesting a conserved role for this microexon in regulating RyR2 function at adult stages.

### Ryr2 Microexon 4 is Controlled by RBFOX2 in Embryonic Mouse Hearts and Cardiomyocytes.

Since Ryr2 microexon 4 is alternatively spliced during heart development, we wondered what controls Ryr2 microexon 4 in the embryonic heart. Microexons are regulated by RNA binding proteins including PTBP and RBFOX families (34-36). PTBP proteins are expressed in endothelial cells, neurons and fibroblasts but not much in cardiomyocytes where RyR2 functions (59). On the other hand, RBFOX2 is highly expressed in cardiomyocytes and is necessary for heart development and function (42). Thus, we checked whether RBFOX2 contributes to Ryr2 microexon 4 regulation in the heart.

We have recently generated a conditional knockout mouse model of Rbfox2 (Rbfox2-CKO) in the embryonic heart (42). Rbfox2 mutant embryos died around mid-gestation, displaying severe congestive heart failure at embryonic day 10.5 (E10.5) (42). To determine whether RBFOX2 regulates Ryr2 microexon 4, we examined our AS data obtained from Rbfox2 mutant embryos at E9.5 before they developed congestive heart failure (42). In this dataset, Ryr2 microexon 4 were more excluded in Rbfox2 mutants.

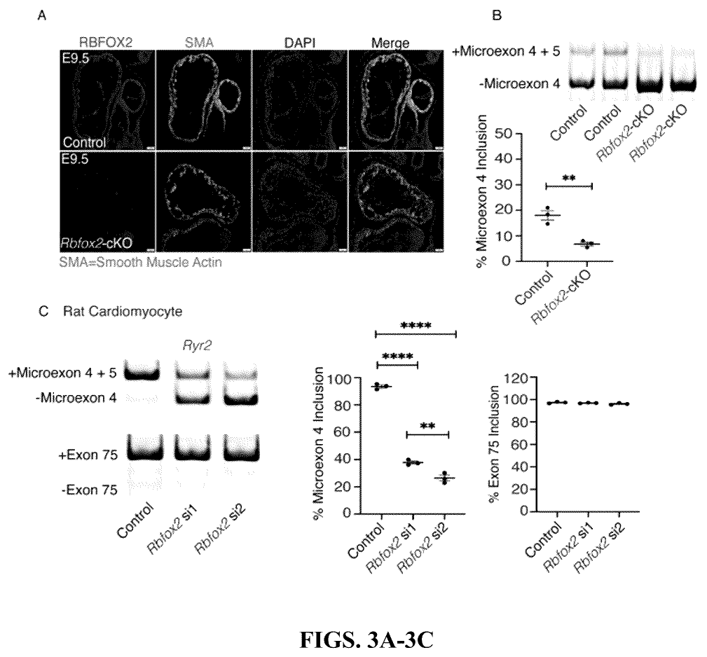

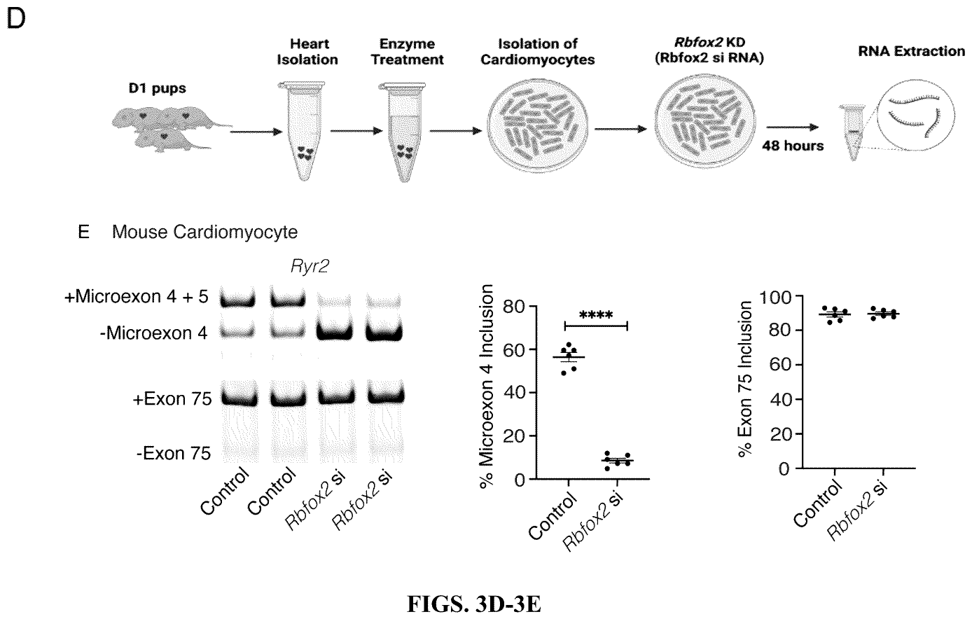

To validate this RNA-seq result, we assessed AS of Ryr2 microexon 4 in control (Rbfox2^{flox/flow}) vs Rbfox2-CKO (Rbfox2^{flox/flow-}; Nkx2-5^{Cre/+}) embryo hearts. Immunofluorescence revealed that RBFOX2 (red) was successfully deleted in the cardiac region marked by alpha smooth muscle actin (SMA) (green) in comparison to control embryos at E9.5 (FIG. 3A, top vs bottom). Ryr2 microexon 4 was almost completely excluded upon Rbfox2 loss in embryo hearts (FIG. 3B), suggesting that this microexon inclusion is regulated by Rbfox2 in embryonic mouse hearts at E9.5 before severe cardiovascular defects occur.

Next, we examined the regulation of Ryr2 microexon 4 inclusion by RBFOX2 in neonatal cardiomyocytes (mouse and rat) treated with scrambled or Rbfox2-specific small interfering RNAs (siRNAs). Ryr2 microexon 4 inclusion decreased from 95% to 30-40% in RBFOX2 depleted neonatal rat cardiomyocytes in comparison to scrambled controls (FIG. 3C). As a control, we checked AS of Ryr2 exon75, which remained unchanged. We also tested whether this regulation occurs in mouse cardiomyocytes, we isolated neonatal cardiomyocytes from one-day-old mice and used siRNAs to knock down Rbfox2, to evaluate microexon 4 inclusion (FIG. 3D). Consistently, Ryr2 microexon 4 inclusion decreased from ˜58% to 5% in RBFOX2 depleted mouse cardiomyocytes (FIG. 3E) similar to that in rat cardiomyocytes and Rbfox2 mutant mice. Importantly, RBFOX2 depletion did not affect AS of exon 75 (FIG. 3E). These results demonstrate that RBFOX2 specifically regulates Ryr2 microexon 4 inclusion in embryonic hearts and in both rat and mouse cardiomyocytes.

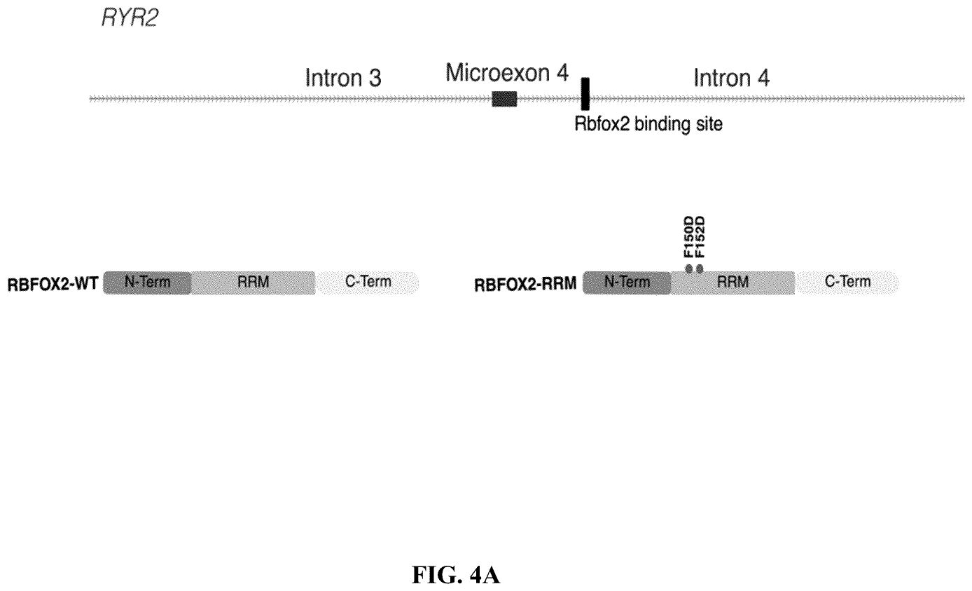

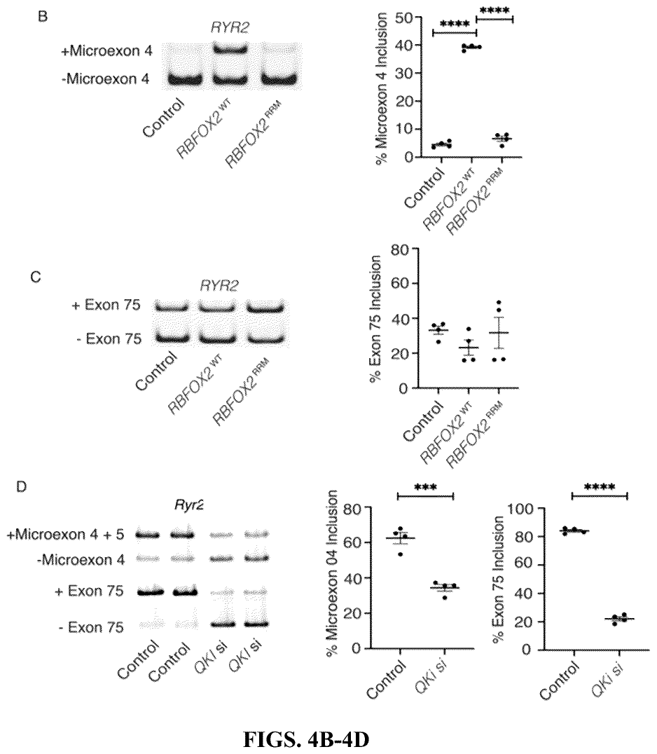

We found RBFOX2 binding sites in intron located downstream of microexon 4 (FIG. 4A). To determine whether RBFOX2 binding activity is necessary for AS of RYR2 microexon 4, we used an RNA binding mutant of RBFOX2 (RBFOX2^{RRM}) with low RNA binding activity (61-63). We induced expression of WT (RBFOX2^{WT}) or RNA binding mutant of RBFOX2 (RBFOX2^{RRM}) in HEK293 Flip-in stable cells, which have very low levels of endogenous RBFOX2 and express RyR2 (42).

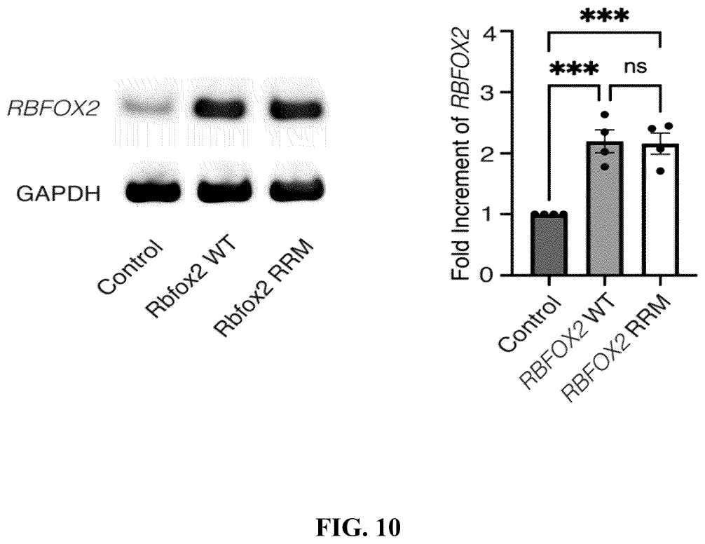

RBFOX2^{WT} induction resulted in increased inclusion of endogenous RYR2 microexon 4 (FIG. 4B) compared to uninduced cells. RBFOX2^{WT} mediated AS changes in RYR2 were reciprocal to the loss of function studies as expected (FIG. 3). Importantly, expression of mutant RBFOX2^{RRM} didn't affect microexon 4 inclusion (FIG. 4B) despite its similar expression levels to RBFOX2^{WT} (FIG. 10). Exon 75 inclusion was not affected in either RBFOX2^{WT} or RBFOX2^{RRM} expressing cells (FIG. 4C). These results show that RBFOX2 RNA binding activity is critical for AS regulation of RYR2 microexon 4.

We also found binding motifs (ACUAAC or ACUAUC) for RNA binding protein Quaking (QKI) in flanking intron 4 of RyR2 microexon 4. To assess its role in microexon 4 splicing, we depleted QKI in mouse cardiomyocytes and found that QKI regulates alternative splicing of both RyR2 exon4 and exon 75 (FIG. 4D). These results indicate that RNA binding proteins RBFOX2 and QKI regulate RyR2 microexon 4 inclusion.

### Ryr2 Microexon 4 Skipping Via Splice Switching Antisense Oligonucleotide Resulted in Lower Intracellular Ca²⁺ Transients and Decreased Cardiomyocyte Beating Rate.

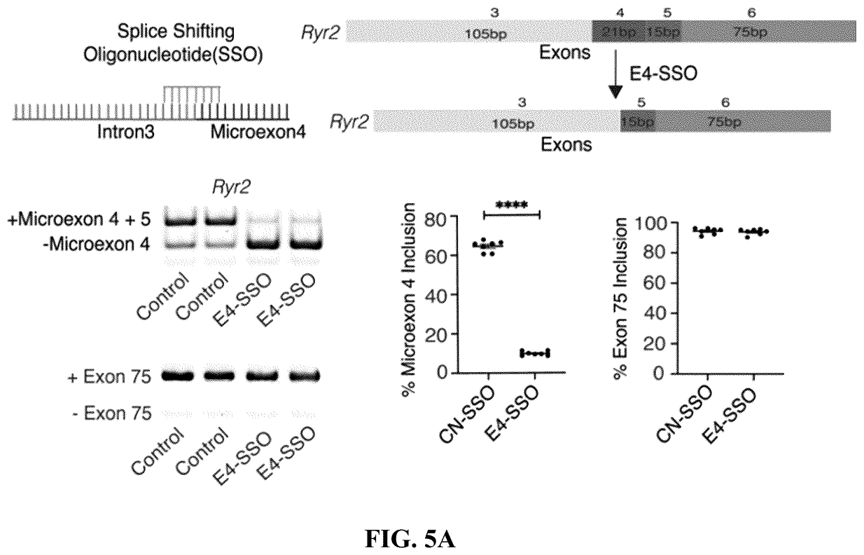

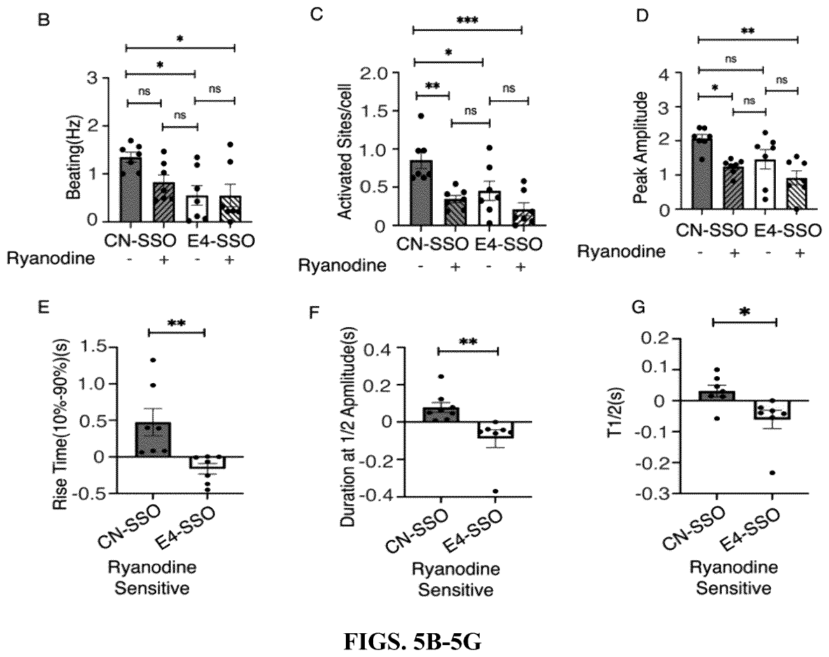

Next, we wanted to test the consequences of Ryr2 microexon 4 exclusion on RyR2 function. To do that, we used Ryr2 microexon4 targeting splice switching oligonucleotides (E4-SSO). Ryr2 E4-SSO is designed to base pair with the 3′ splice site of microexon 4 to prevent spliceosome interactions, causing specific exclusion of this microexon in Ryr2 transcripts (FIG. 5A). As a control, we used GFP targeting SSO (Control SSO). We transfected neonatal mouse cardiomyocytes with control or Ryr2 microexon targeting SSOs (E4-SSO). We used high-speed spinning disk confocal imaging and fluo-4 AM, a Ca²⁺ indicator, to record Ca²⁺ transients in cardiomyocytes before and after treating with Ryanodine (1 microM), which selectively blocks RYR channels.

E4-SSO treatment successfully resulted in Ryr2 microexon 4 exclusion (FIG. 5A). To ensure that this SSO is specific to Ryr2 microexon 4 exclusion, we also examined exon 75 exclusion. We found that Ryr2 exon 75 AS was unaffected in E4-SSO treated cardiomyocytes, indicating that E4-SSO was specific to microexon 4 and did not alter Ryr2 mRNA levels or AS of other Ryr2 exon (FIG. 5A). Notably, Ryr2 microexon 4 excluding cardiomyocytes exhibited lower beating rate and lower basal activity of Ca²⁺ transients when compared to controls (FIGS. 5B, 5C). The basal activity of Ca²⁺ transients was reduced by ryanodine in control but not in E4-SSO cardiomyocytes, indicating a decrease in the activity of RyR-mediated Ca²⁺ transients upon Ryr2 microexon4 exclusion. Ryanodine reduced the amplitude of basal Ca²⁺ transients in control but not in E4-SSO cardiomyocytes, suggesting a reduced contribution of RyRs to basal Ca²⁺ transients in Ryr2 microexon 4 excluding cardiomyocytes (FIG. 5D). We then compared the kinetic properties of RyR-mediated Ca²⁺ transients (ryanodine-sensitive Ca²⁺ transients) between the two groups of cardiomyocytes. The rise time (from 10% to 90% of the peak), duration (full width at half maximum amplitude), decay time (to half maximum amplitude) of RyR-mediated Ca²⁺ transients were reduced in E4-SSO cardiomyocytes compared to control cardiomyocytes (FIGS. 5E-5G).

These results demonstrate that exclusion of Ryr2 microexon 4 dampened spontaneous beating and RYR-mediated Ca²⁺ transients. Reduced number and durations of RyR Ca²⁺ transients in Ryr2 microexon 4 excluding cardiomyocytes may indicate partial channel closure or reduced open state probability.

### Ryr2 Microexon 4 Exclusion Renders RyR2 Unresponsive to Caffeine Mediated Ca²⁺ Release

So far, our findings point out that microexon 4 exclusion reduces RyR2 channel activity. To investigate this possibility further, we used caffeine, which triggers the release of Ca²⁺ from SR leading to calcium overload in the cytoplasm (64). We also used ryanodine to specifically block RYRs, followed by treatment with caffeine to confirm caffeine increases RyR-mediated Ca²⁺ transients.

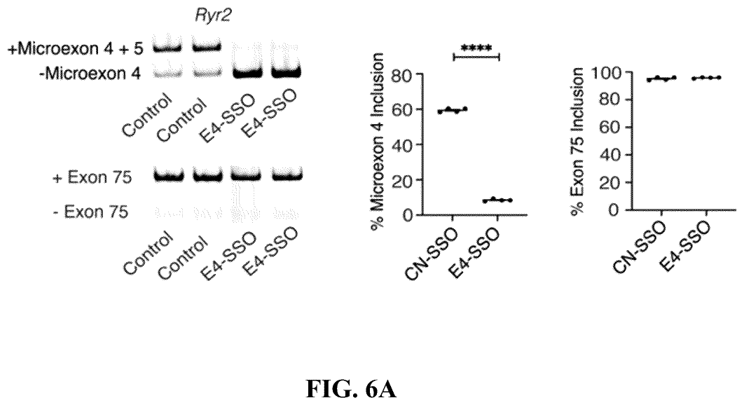

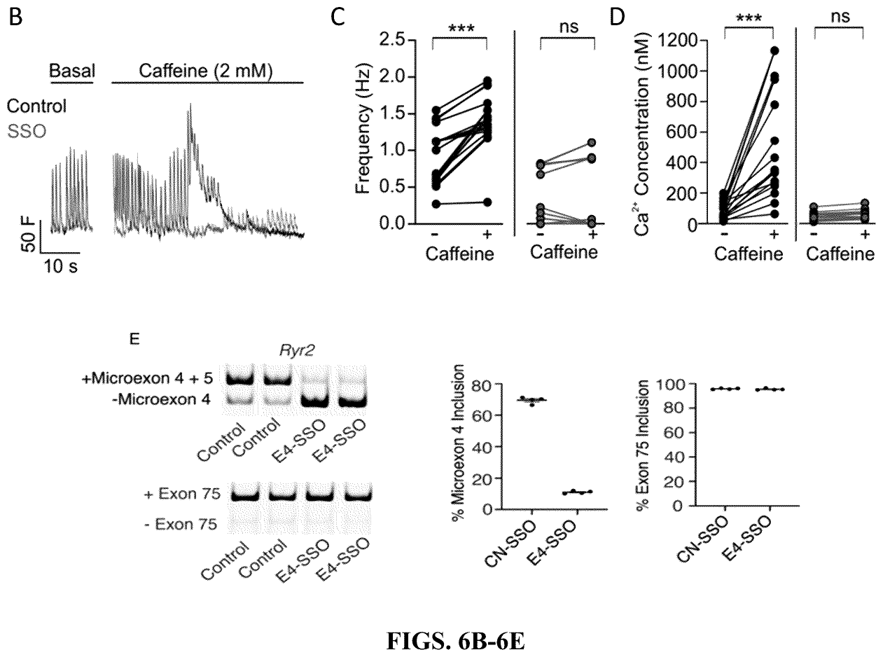

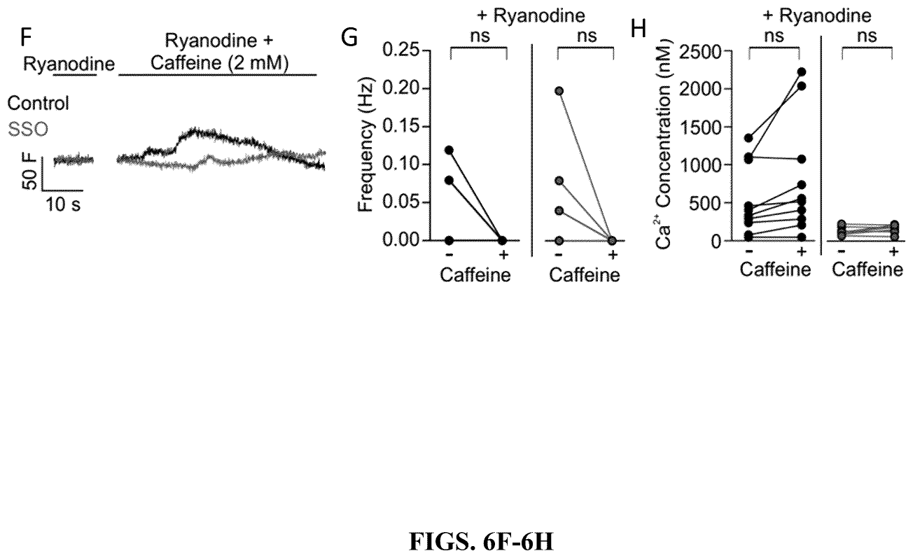

We treated neonatal mouse cardiomyocytes with control or E4-SSOs. Microexon 4 targeting SSO successfully caused almost complete Ryr2 microexon 4 exclusion (FIG. 6A). Caffeine treatment induced an increase in the frequency of Ca²⁺ transients and cellular Ca²⁺ concentration in control cardiomyocytes as expected but not in Ryr2 microexon excluding cardiomyocytes (FIGS. 6B-6D).

We treated neonatal mouse cardiomyocytes expressing control or E4-SSOs with ryanodine first to selectively block the channel and then added caffeine to stimulate Ca²⁺ release. Microexon 4 targeting SSO successfully caused Ryr2 microexon 4 skipping (FIG. 6E). Caffeine-induced increase in Ca²⁺ transients was abolished in the presence of ryanodine (FIGS. 6G, 6H), suggesting that caffeine-induced Ca²⁺ transients reflect RyR activity in Ryr2 microexon 4 excluded cardiomyocytes (FIGS. 6G, 6H). These results indicate that microexon 4 exclusion reduces the activity of RyR2-mediated Ca²⁺ transients, resulting in low beating rate of cardiomyocytes.

### L-Type Ca2+ Channel-Induced RyR Ca²⁺ Sparks in Mouse Cardiomyocytes are Impaired Upon Microexon 4 Skipping

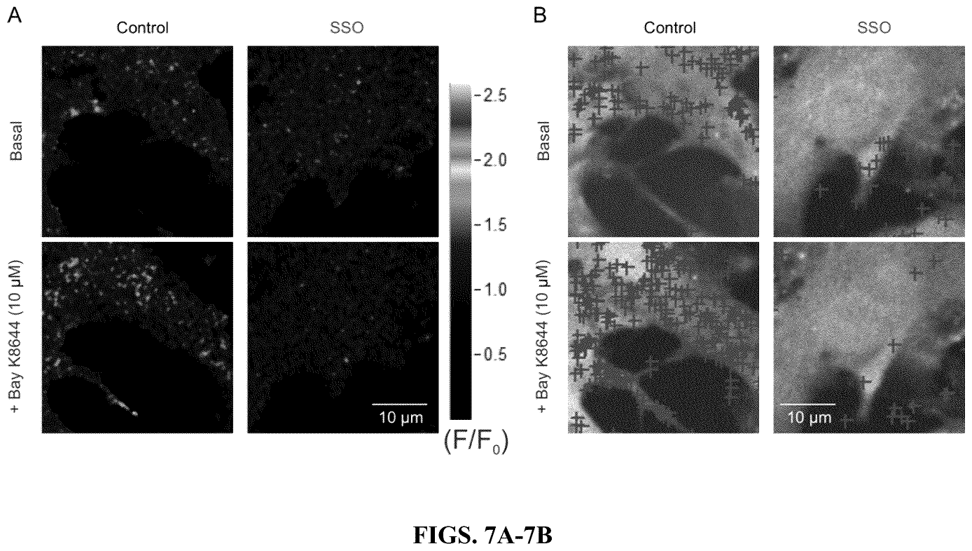

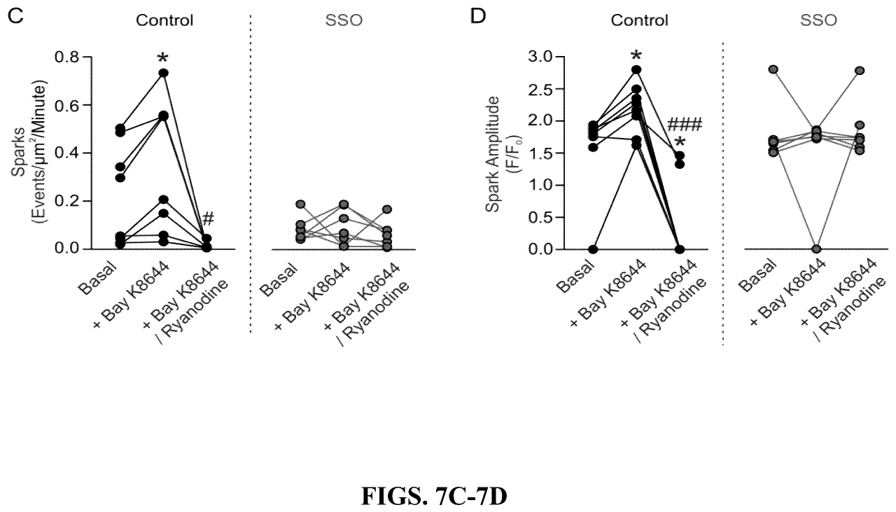

To determine whether L-type Ca2+ channel-induced RyR Ca²⁺ sparks are affected when microexon 4 is skipped, we activated L-type calcium channel using Bay K8644 and treated these cells with low dose of Ryanodine. In control SSO treated cardiomyocytes, calcium sparks and amplitude were increased after activating L-type channel (+Bay K8644) (FIG. 7A, 7B). As expected, Ryanodine treatment reduced calcium sparks induced by Bay K8644 (FIG. 7A,7B,7C,7D: black). In E4-SSO treated cardiomyocytes that cause microexon 4 skipping, Bay K8644 did not significantly induce calcium sparks and amplitude (FIG. 7A,7B,7C,7D: red). These results indicate that microexon 4 inclusion is important for RyR calcium sparks induced by L-type calcium channel activation.

Identification of SSOs that Cause Human RyR2 Microexon 4 Skipping by Targeting Splice Site and RNA Binding Protein Binding Sites

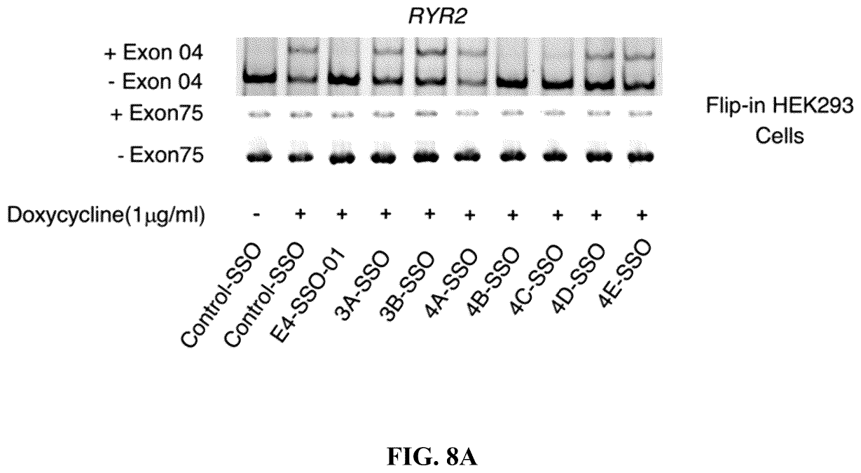

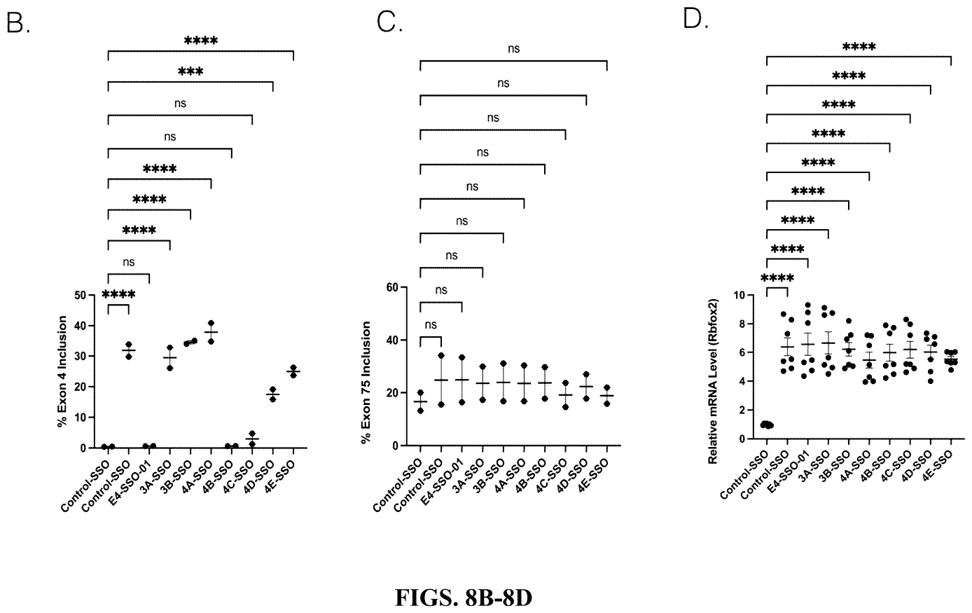

Since microexon 4 has an important role in controlling RyR2 activity, we wanted to identify how this microexon splicing is regulated and find SSOs that can cause microexon 4 skipping of human RyR2. We used 8 different SSOs that base pair intronic regions flanking upstream and downstream of human RyR2 microexon 4. RYR2 is expressed in HEK293 cells but microexon 4 is excluded. For that reason, we induced expression of RBFOX2 to promote microexon 4 inclusion and then treated these cells with these 8 SSOs. We found that 3 SSOs effectively caused microexon 4 exclusion in RBFOX2 expressing cells (FIG. 8A, 8B). These 3 SSOs blocked either 5′ splice site, RBFOX2 or QKI binding sites. These SSOs did not affect exon 75 levels or splicing (FIG. 8A, 8C), indicating their specific effects on microexon 4. Importantly, RBFOX2 levels were the same in these cells treated with SSOs (FIG. 8D), showing that the exon exclusion effect of these 3 SSOs are not due to lower expression of RBFOX2 levels.

### Discussion

Mutations or dysregulation of RyR2 leads to various cardiac abnormalities including catecholaminergic polymorphic ventricular tachycardia, idiopathic ventricular fibrillation, atrial fibrillation, heart failure, and cardiomyopathies (7,21,65-81). RYR2 gain of function mutations result in excessive calcium leak resulting in tachycardia and sudden death. Therefore, RyR2 is a major target for treatment of many heart conditions (82-84). RyR2 has complex regulation. Our study reveals a novel post-transcriptional switch mechanism mediated by inclusion/exclusion of a microexon that impact RyR2 channel activity during heart development. Our work has future therapeutic implications by providing Ryr2 targeting SSO that can base pair with endogenous Ryr2 pre-mRNA and influence RyR2 channel activity.

Our findings demonstrate a molecular switch in Ryr2 pre-mRNA that controls microexon 4 inclusion at the onset of T-tubule formation in the developing heart. We found that Ryr2 microexon 4 inclusion coincided with the timing of T-tubule formation and maturation (FIG. 2) during heart development (25,85). We propose that microexon 4 inclusion is necessary to increase RyR2 Ca²⁺ release activity for efficient cardiac contraction cycle in postnatal and adult hearts. We find that at embryonic stages both microexon 4 including and excluding Ryr2 isoforms co-exist. The stoichiometry of these different RyR2 isoforms with and without microexon in the homotetrameric structure of RyR2 is not known. N-terminal domain of RyR2 that forms inter and intra-subunit interactions within the tetrameric structure is necessary for pore's opening and closing (39). It is possible that RyR2 microexon 4 lacking isoforms form a structurally different homotetramer and display different channel activity than purely microexon 4 containing RyR2 isoforms. Structural analysis of different RyR2 isoforms might provide insights. It is also intriguing that RyR2 isoform switch closely follows T-tubule formation and maturation. It may be that changes in the cytoplasmic membrane during T-tubule formation/maturation somehow signals Rbfox2 to control Ryr2 microexon splicing and generate different RyR2 isoform. These questions need further investigations to be answered.

We generated SSO that can successfully cause Ryr2 microexon 4 skipping in cardiomyocytes. Notably, in the absence of this microexon, RyR2 was immune to caffeine-induced Ca²⁺ release (FIGS. 5 and 6). Since the N-terminus domain (where microexon resides) is important for pore's opening and closing (39) and microexon encoded amino acids are located at the dimer interface of the tetrameric channel near the pore (FIG. 1), we propose that microexon 4 skipping may contribute to RyR2 channel pore opening or closure. Structural studies are needed to confirm this. A study identified two RYR2 spliced variants generated via insertion of small (30 bp and 24 bp) sequences at the C-terminal part of the protein (86) and another study showed that these isoforms when overexpressed in HL-1 cells have roles in apoptosis (87). These studies combined with our findings suggest that Ryr2 pre-mRNA gives rise to different Ryr2 mRNA isoforms with unique functions.

We identified RNA binding proteins RBFOX2 and QKI as regulators of Ryr2 microexon 4 AS (FIGS. 3 and 4). RBFOX2 is implicated in many cardiovascular diseases and has roles in EC coupling in the heart, arrythmias and mitochondrial health (88-92). Rbfox2 conditional knockout mouse model that developed congestive heart failure at embryonic stages and died in utero soon after (42). RyR2 microexon 4 was more excluded in Rbfox2 mutant mouse hearts. Similarly, Rbfox2 depletion in both mouse and rat cardiomyocytes also caused more microexon 4 skipping. Our results add another layer of regulation to the complex RyR2 biology demonstrating that AS of RyR2 is tightly regulated during development by RNA binding proteins. Importantly, SSOs that base pair with RBFOX2 and QKI binding sites on the RyR2 intron caused microexon 4 exclusion, suggesting that these proteins promote RyR2 microexon 4 inclusion. Knockdown experiments combined with SSOs validate the importance of these RNA binding proteins in regulating human RyR2 microexon 4 splicing.

In summary, our study demonstrated the role of RyR2 microexon 4 in regulating the channel's calcium release activity. Our work has implications to ultimately control the heart rate in patients with overactive RyR2 channel in the future. The successful use of our antisense splice switching oligos to exclude this microexon may help reduce abnormal heart rate in arrythmia patients and thereby may protect them from long term cardiac complications. Notably, antisense oligonucleotides are FDA approved (93,94) and represent a promising therapeutic approach targeting RYR2 pre-mRNA. It is important to note that microexon 4 is conserved between mouse and human. We have identified at least 3 SSOs that can cause microexon 4 exclusion in human RyR2. Future efficacy and safety studies with these SSO targeting human RYR2 microexon 4 in preclinical disease models could pave the way for innovative therapeutic approach in treating cardiac arrhythmia.

### REFERENCES

1. Bers, D. M. (2002) Cardiac excitation-contraction coupling. Nature, 415, 198-205.
2. Li, Y., Wei, J., Guo, W., Sun, B., Estillore, J. P., Wang, R., Yoruk, A., Roston, T. M., Sanatani, S., Wilde, A. A. M. et al. (2021) Human RyR2 (Ryanodine Receptor 2) Loss-of-Function Mutations: Clinical Phenotypes and In Vitro Characterization. Circ Arrhythm Electrophysiol, 14, e010013.
3. Vemireddy, L. P., Aqeel, A., Ying, G. W., Majlesi, D. and Woo, V. (2021) A Rare Case of RYR2 Mutation Causing Sudden Cardiac Arrest Due to Catecholaminergic Polymorphic Ventricular Tachycardia. Cureus, 13, e13417.
4. Jiang, D., Xiao, B., Yang, D., Wang, R., Choi, P., Zhang, L., Cheng, H. and Chen, S. R. (2004) RyR2 mutations linked to ventricular tachycardia and sudden death reduce the threshold for store-overload-induced Ca2+ release (SOICR). Proc Natl Acad Sci USA, 101, 13062-13067.
5. Steinberg, C., Roston, T. M., van der Werf, C., Sanatani, S., Chen, S. R. W., Wilde, A. A. M. and Krahn, A. D. (2023) RYR2-ryanodinopathies: from calcium overload to calcium deficiency. Europace, 25.
6. Xing, Y., Cui, T. and Sun, F. (2024) A novel RyR2 mutation associated with co-morbid catecholaminergic polymorphic ventricular tachycardia (CPVT) and benign epilepsy with centrotemporal spikes (BECTS). J. Electrocardiol, 84, 75-80.
7. Medeiros-Domingo, A., Bhuiyan, Z. A., Tester, D. J., Hofman, N., Bikker, H., van Tintelen, J. P., Mannens, M. M., Wilde, A. A. and Ackerman, M. J. (2009) The RYR2-encoded ryanodine receptor/calcium release channel in patients diagnosed previously with either catecholaminergic polymorphic ventricular tachycardia or genotype negative, exercise-induced long QT syndrome: a comprehensive open reading frame mutational analysis. J Am Coll Cardiol, 54, 2065-2074.
8. Rampazzo, A. (2006) Genetic bases of arrhythmogenic right ventricular Cardiomyopathy. Heart Int, 2, 17.
9. Dokuparti, M. V. N., Pamuru, P. R., Thakkar, B., Tanjore, R. R. and Nallari, P. (2005) Etiopathogenesis of arrhythmogenic right ventricular cardiomyopathy. J Hum Genet, 50, 375-381.
10. Ormerod, J. O. M., Ormondroyd, E., Li, Y., Taylor, J., Wei, J., Guo, W., Wang, R., Sarton, C. N. S., McGuire, K., Dreau, H. M. P. et al. (2022) Provocation Testing and Therapeutic Response in a Newly Described Channelopathy: RyR2 Calcium Release Deficiency Syndrome. Circ Genom Precis Med, 15, e003589.
11. Roston, T. M., Wei, J., Guo, W., Li, Y., Zhong, X., Wang, R., Estillore, J. P., Peltenburg, P. J., Noguer, F. R. I., Till, J. et al. (2022) Clinical and Functional Characterization of Ryanodine Receptor 2 Variants Implicated in Calcium-Release Deficiency Syndrome. JAMA Cardiol, 7, 84-92.
12. Sun, B., Yao, J., Ni, M., Wei, J., Zhong, X., Guo, W., Zhang, L., Wang, R., Belke, D., Chen, Y. X. et al. (2021) Cardiac ryanodine receptor calcium release deficiency syndrome. Sci Transl Med, 13.
13. Costa, S., Medeiros-Domingo, A., Gasperetti, A., Breitenstein, A., Steffel, J., Guidetti, F., Flammer, A., Odening, K., Ruschitzka, F., Duru, F. et al. (2021) Familial dilated cardiomyopathy associated with a novel heterozygous RYR2 early truncating variant. Cardiol J, 28, 173-175.
14. Jona, I. and Nanasi, P. P. (2006) Cardiomyopathies and sudden cardiac death caused by RyR2 mutations: are the channels the beginning and the end? Cardiovasc Res, 71, 416-418.
15. Duvekot, J. C., Baas, A. F., Volker-Touw, C. M. L., Bikker, H., Schroer, C. and Breur, J. (2021) Early Lethal Noncompaction Cardiomyopathy in Siblings With Compound Heterozygous RYR2 Variant. Can J Cardiol, 37, 1864-1866.
16. Dridi, H., Liu, Y., Reiken, S., Liu, X., Argyrousi, E. K., Yuan, Q., Miotto, M. C., Sittenfeld, L., Meddar, A., Soni, R. K. et al. (2023) Heart failure-induced cognitive dysfunction is mediated by intracellular Ca(2+) leak through ryanodine receptor type 2. Nat Neurosci, 26, 1365-1378.
17. Dubois, M., Boulghobra, D., Rochebloine, G., Pallot, F., Yehya, M., Bornard, I., Gayrard, S., Coste, F., Walther, G., Meyer, G. et al. (2024) Hyperglycemia triggers RyR2-dependent alterations of mitochondrial calcium homeostasis in response to cardiac ischemia-reperfusion: Key role of DRP1 activation. Redox Biol, 70, 103044.
18. Dillmann, W. H. (2019) Diabetic Cardiomyopathy. Circ Res, 124, 1160-1162.
19. Boehm, B. M., Gaa, J., Hoppmann, P., Martens, E. and Westphal, D. S. (2023) The Role of RYR2 in Atrial Fibrillation. Case Rep Cardiol, 2023, 6555998.
20. Dobrev, D. and Wehrens, X. H. (2014) Role of RyR2 phosphorylation in heart failure and arrhythmias: Controversies around ryanodine receptor phosphorylation in cardiac disease. Circ Res, 114, 1311-1319; discussion 1319.
21. Di Pino, A., Caruso, E., Costanzo, L. and Guccione, P. (2014) A novel RyR2 mutation in a 2-year-old baby presenting with atrial fibrillation, atrial flutter, and atrial ectopic tachycardia. Heart Rhythm, 11, 1480-1483.
22. Nakai, J., Imagawa, T., Hakamat, Y., Shigekawa, M., Takeshima, H. and Numa, S. (1990) Primary structure and functional expression from cDNA of the cardiac ryanodine receptor/calcium release channel. FEBS Lett, 271, 169-177.
23. Otsu, K., Willard, H. F., Khanna, V. K., Zorzato, F., Green, N. M. and MacLennan, D. H. (1990) Molecular cloning of cDNA encoding the Ca2+ release channel (ryanodine receptor) of rabbit cardiac muscle sarcoplasmic reticulum. J Biol Chem, 265, 13472-13483.
24. Brette, F. and Orchard, C. (2003) T-tubule function in mammalian cardiac myocytes. Circ Res, 92, 1182-1192.
25. Liu, C., Spinozzi, S., Chen, J. Y., Fang, X., Feng, W., Perkins, G., Cattaneo, P., Guimaraes-Camboa, N., Dalton, N. D., Peterson, K. L. et al. (2019) Nexilin Is a New Component of Junctional Membrane Complexes Required for Cardiac T-Tubule Formation. Circulation, 140, 55-66.
26. Takeshima, H., Komazaki, S., Hirose, K., Nishi, M., Noda, T. and Iino, M. (1998) Embryonic lethality and abnormal cardiac myocytes in mice lacking ryanodine receptor type 2. EMBO J, 17, 3309-3316.
27. Okolo, C. A., Khaing, E. P., Mereacre, V., Wallace, R. S., Munro, M. L., Erickson, J. R. and Jones, P. P. (2023) Direct regulation of the cardiac ryanodine receptor (RyR2) by 0-GlcNAcylation. Cardiovasc Diabetol, 22, 276.
28. Waddell, H. M. M., Zhang, J. Z., Hoeksema, K. J., McLachlan, J. J., McLay, J. C. and Jones, P. P. (2016) Oxidation of RyR2 Has a Biphasic Effect on the Threshold for Store Overload-Induced Calcium Release. Biophys J, 110, 2386-2396.
29. Gonano, L. A., Aitken-Buck, H. M., Chakraborty, A. D., Worthington, L. P. I., Cully, T. R., Lamberts, R. R., Vila-Petroff, M. G. and Jones, P. P. (2022) Regulation of cardiac ryanodine receptor function by the cyclic-GMP dependent protein kinase G. Curr Res Physiol, 5, 171-178.
30. Ustianenko, D., Weyn-Vanhentenryck, S. M. and Zhang, C. (2017) Microexons: discovery, regulation, and function. Wiley Interdiscip Rev RNA, 8.
31. Irimia, M., Weatheritt, R. J., Ellis, J. D., Parikshak, N. N., Gonatopoulos-Pournatzis, T., Babor, M., Quesnel-Vallieres, M., Tapial, J., Raj, B., O'Hanlon, D. et al. (2014) A highly conserved program of neuronal microexons is misregulated in autistic brains. Cell, 159, 1511-1523.
32. Gonatopoulos-Pournatzis, T. and Blencowe, B. J. (2020) Microexons: at the nexus of nervous system development, behaviour and autism spectrum disorder. Curr Opin Genet Dev, 65, 22-33.
33. Volfovsky, N., Haas, B. J. and Salzberg, S. L. (2003) Computational discovery of internal micro-exons. Genome Res, 13, 1216-1221.
34. Berget, S. M. (1995) Exon recognition in vertebrate splicing. J Biol Chem, 270, 2411-2414.
35. Dominski, Z. and Kole, R. (1991) Selection of splice sites in pre-mRNAs with short internal exons. Mol Cell Biol, 11, 6075-6083.
36. Black, D. L. (1991) Does steric interference between splice sites block the splicing of a short c-src neuron-specific exon in non-neuronal cells? Genes Dev, 5, 389-402.
37. Li, Y. I., Sanchez-Pulido, L., Haerty, W. and Ponting, C. P. (2015) RBFOX and PTBP1 proteins regulate the alternative splicing of micro-exons in human brain transcripts. Genome Res, 25, 1-13.
38. Cooper, T. A. and Ordahl, C. P. (1985) A single cardiac troponin T gene generates embryonic and adult isoforms via developmentally regulated alternate splicing. J Biol Chem, 260, 11140-11148.
39. Seidel, M., de Meritens, C. R., Johnson, L., Parthimos, D., Bannister, M., Thomas, N. L., Ozekhome-Mike, E., Lai, F. A. and Zissimopoulos, S. (2021) Identification of an amino-terminus determinant critical for ryanodine receptor/Ca2+ release channel function. Cardiovasc Res, 117, 780-791.
40. Lobo, P. A., Kimlicka, L., Tung, C. C. and Van Petegem, F. (2011) The deletion of exon 3 in the cardiac ryanodine receptor is rescued by beta strand switching. Structure, 19, 790-798.
41. Moses, K. A., DeMayo, F., Braun, R. M., Reecy, J. L. and Schwartz, R. J. (2001) Embryonic expression of an Nkx2-5/Cre gene using ROSA26 reporter mice. Genesis, 31, 176-180.
42. Verma, S. K., Deshmukh, V., Thatcher, K., Belanger, K. K., Rhyner, A. M., Meng, S., Holcomb, R. J., Bressan, M., Martin, J. F., Cooke, J. P. et al. (2022) RBFOX2 is required for establishing RNA regulatory networks essential for heart development. Nucleic Acids Research, 50, 2270-2286.
43. Gong, D., Chi, X., Wei, J., Zhou, G., Huang, G., Zhang, L., Wang, R., Lei, J., Chen, S. R. W. and Yan, N. (2019) Modulation of cardiac ryanodine receptor 2 by calmodulin. Nature, 572, 347-351.
44. Nutter, C. A., Jaworski, E. A., Verma, S. K., Deshmukh, V., Wang, Q., Botvinnik, O. B., Lozano, M. J., Abass, I. J., Ijaz, T., Brasier, A. R. et al. (2016) Dysregulation of RBFOX2 Is an Early Event in Cardiac Pathogenesis of Diabetes. Cell Rep, 15, 2200-2213.
45. Verma, S. K., Deshmukh, V., Liu, P., Nutter, C. A., Espejo, R., Hung, M. L., Wang, G. S., Yeo, G. W. and Kuyumcu-Martinez, M. N. (2013) Reactivation of fetal splicing programs in diabetic hearts is mediated by protein kinase C signaling. J Biol Chem, 288, 35372-35386.
46. Chen, Y. L., Daneva, Z., Kuppusamy, M., Ottolini, M., Baker, T. M., Klimentova, E., Shah, S. A., Sokolowski, J. D., Park, M. S. and Sonkusare, S. K. (2022) Novel Smooth Muscle Ca(2+)-Signaling Nanodomains in Blood Pressure Regulation. Circulation, 146, 548-564.
47. Maravall, M., Mainen, Z. F., Sabatini, B. L. and Svoboda, K. (2000) Estimating intracellular calcium concentrations and buffering without wavelength ratioing. Biophys J, 78, 2655-2667.
48. Waterhouse, A. M., Procter, J. B., Martin, D. M., Clamp, M. and Barton, G. J. (2009) Jalview Version 2—a multiple sequence alignment editor and analysis workbench. Bioinformatics, 25, 1189-1191.
49. Brett D. Scott, P. D., Mike Martin, D M. (2016) UNDERSTANDING VITAL LIFE SIGNS IN HORSES.
50. Kimlicka, L., Lau, K., Tung, C. C. and Van Petegem, F. (2013) Disease mutations in the ryanodine receptor N-terminal region couple to a mobile intersubunit interface. Nat Commun, 4, 1506.
51. Tung, C. C., Lobo, P. A., Kimlicka, L. and Van Petegem, F. (2010) The amino-terminal disease hotspot of ryanodine receptors forms a cytoplasmic vestibule. Nature, 468, 585-588.
52. Seidel, M., Thomas, N. L., Williams, A. J., Lai, F. A. and Zissimopoulos, S. (2015) Dantrolene rescues aberrant N-terminus intersubunit interactions in mutant pro-arrhythmic cardiac ryanodine receptors. Cardiovasc Res, 105, 118-128.
53. Zissimopoulos, S., Marsh, J., Stannard, L., Seidel, M. and Lai, F. A. (2014) N-terminus oligomerization is conserved in intracellular calcium release channels. Biochem J, 459, 265-273.
54. Amador, F. J., Kimlicka, L., Stathopulos, P. B., Gasmi-Seabrook, G. M., Maclennan, D. H., Van Petegem, F. and Ikura, M. (2013) Type 2 ryanodine receptor domain A contains a unique and dynamic alpha-helix that transitions to a beta-strand in a mutant linked with a heritable cardiomyopathy. J Mol Biol, 425, 4034-4046.
55. Lai, F. A., Dent, M., Wickenden, C., Xu, L., Kumari, G., Misra, M., Lee, H. B., Sar, M. and Meissner, G. (1992) Expression of a cardiac Ca(2+)-release channel isoform in mammalian brain. Biochem J, 288 (Pt 2), 553-564.
56. Nakanishi, S., Kuwajima, G. and Mikoshiba, K. (1992) Immunohistochemical localization of ryanodine receptors in mouse central nervous system. Neurosci Res, 15, 130-142.
57. Sharp, A. H., McPherson, P. S., Dawson, T. M., Aoki, C., Campbell, K. P. and Snyder, S. H. (1993) Differential immunohistochemical localization of inositol 1,4,5-trisphosphate- and ryanodine-sensitive Ca2+ release channels in rat brain. J Neurosci, 13, 3051-3063.
58. Furuichi, T., Furutama, D., Hakamata, Y., Nakai, J., Takeshima, H. and Mikoshiba, K. (1994) Multiple types of ryanodine receptor/Ca2+ release channels are differentially expressed in rabbit brain. J Neurosci, 14, 4794-4805.
59. Liu, H., Duan, R., He, X., Qi, J., Xing, T., Wu, Y., Zhou, L., Wang, L., Shao, Y., Zhang, F. et al. (2023) Endothelial deletion of PTBP1 disrupts ventricular chamber development. Nat Commun, 14, 1796.
60. Yeo, G. W., Coufal, N. G., Liang, T. Y., Peng, G. E., Fu, X. D. and Gage, F. H. (2009) An RNA code for the FOX2 splicing regulator revealed by mapping RNA-protein interactions in stem cells. Nat Struct Mol Biol, 16, 130-137.
61. Auweter, S. D., Fasan, R., Reymond, L., Underwood, J. G., Black, D. L., Pitsch, S. and Allain, F. H. (2006) Molecular basis of RNA recognition by the human alternative splicing factor Fox-1. EMBO J, 25, 163-173.
62. Mayeda, A., Munroe, S. H., Caceres, J. F. and Krainer, A. R. (1994) Function of conserved domains of hnRNP A1 and other hnRNP A/B proteins. EMBO J, 13, 5483-5495.
63. Sun, S., Zhang, Z., Fregoso, O. and Krainer, A. R. (2012) Mechanisms of activation and repression by the alternative splicing factors RBFOX1/2. RNA, 18, 274-283.
64. Balasubramaniam, R., Chawla, S., Grace, A. A. and Huang, C. L. (2005) Caffeine-induced arrhythmias in murine hearts parallel changes in cellular Ca(2+) homeostasis. Am J Physiol Heart Circ Physiol, 289, H1584-1593.
65. Bers, D. M. (2014) Cardiac sarcoplasmic reticulum calcium leak: basis and roles in cardiac dysfunction. Annu Rev Physiol, 76, 107-127.
66. George, C. H., Jundi, H., Thomas, N. L., Fry, D. L. and Lai, F. A. (2007) Ryanodine receptors and ventricular arrhythmias: emerging trends in mutations, mechanisms and therapies. J Mol Cell Cardiol, 42, 34-50.
67. Mohamed, U., Napolitano, C. and Priori, S. G. (2007) Molecular and electrophysiological bases of catecholaminergic polymorphic ventricular tachycardia. J Cardiovasc Electrophysiol, 18, 791-797.
68. Priori, S. G. and Chen, S. R. (2011) Inherited dysfunction of sarcoplasmic reticulum Ca2+ handling and arrhythmogenesis. Circ Res, 108, 871-883.
69. Priori, S. G., Napolitano, C., Tiso, N., Memmi, M., Vignati, G., Bloise, R., Sorrentino, V. and Danieli, G. A. (2001) Mutations in the cardiac ryanodine receptor gene (hRyR2) underlie catecholaminergic polymorphic ventricular tachycardia. Circulation, 103, 196-200.
70. Laitinen, P. J., Brown, K. M., Piippo, K., Swan, H., Devaney, J. M., Brahmbhatt, B., Donarum, E. A., Marino, M., Tiso, N., Viitasalo, M. et al. (2001) Mutations of the cardiac ryanodine receptor (RyR2) gene in familial polymorphic ventricular tachycardia. Circulation, 103, 485-490.
71. Priori, S. G., Napolitano, C., Memmi, M., Colombi, B., Drago, F., Gasparini, M., DeSimone, L., Coltorti, F., Bloise, R., Keegan, R. et al. (2002) Clinical and molecular characterization of patients with catecholaminergic polymorphic ventricular tachycardia. Circulation, 106, 69-74.
72. Bhuiyan, Z. A., van den Berg, M. P., van Tintelen, J. P., Bink-Boelkens, M. T., Wiesfeld, A. C., Alders, M., Postma, A. V., van Langen, I., Mannens, M. M. and Wilde, A. A. (2007) Expanding spectrum of human RYR2-related disease: new electrocardiographic, structural, and genetic features. Circulation, 116, 1569-1576.
73. Marjamaa, A., Laitinen-Forsblom, P., Wronska, A., Toivonen, L., Kontula, K. and Swan, H. (2011) Ryanodine receptor (RyR2) mutations in sudden cardiac death: studies in extended pedigrees and phenotypic characterization in vitro. Int J Cardiol, 147, 246-252.
74. Ohno, S., Omura, M., Kawamura, M., Kimura, H., Itoh, H., Makiyama, T., Ushinohama, H., Makita, N. and Horie, M. (2014) Exon 3 deletion of RYR2 encoding cardiac ryanodine receptor is associated with left ventricular non-compaction. Europace, 16, 1646-1654.
75. Bauce, B., Rampazzo, A., Basso, C., Bagattin, A., Daliento, L., Tiso, N., Turrini, P., Thiene, G., Danieli, G. A. and Nava, A. (2002) Screening for ryanodine receptor type 2 mutations in families with effort-induced polymorphic ventricular arrhythmias and sudden death: early diagnosis of asymptomatic carriers. J Am Coll Cardiol, 40, 341-349.
76. Tester, D. J., Spoon, D. B., Valdivia, H. H., Makielski, J. C. and Ackerman, M. J. (2004) Targeted mutational analysis of the RyR2-encoded cardiac ryanodine receptor in sudden unexplained death: a molecular autopsy of 49 medical examiner/coroner's cases. Mayo Clin Proc, 79, 1380-1384.
77. d'Amati, G., Bagattin, A., Bauce, B., Rampazzo, A., Autore, C., Basso, C., King, K., Romeo, M. D., Gallo, P., Thiene, G. et al. (2005) Juvenile sudden death in a family with polymorphic ventricular arrhythmias caused by a novel RyR2 gene mutation: evidence of specific morphological substrates. Hum Pathol, 36, 761-767.
78. Nishio, H., Iwata, M. and Suzuki, K. (2006) Postmortem molecular screening for cardiac ryanodine receptor type 2 mutations in sudden unexplained death: R420W mutated case with characteristics of status thymico-lymphatics. Circ J, 70, 1402-1406.
79. Pizzale, S., Gollob, M. H., Gow, R. and Birnie, D. H. (2008) Sudden death in a young man with catecholaminergic polymorphic ventricular tachycardia and paroxysmal atrial fibrillation. J Cardiovasc Electrophysiol, 19, 1319-1321.
80. Zhabyeyev, P., Hiess, F., Wang, R., Liu, Y., Wayne Chen, S. R. and Oudit, G. Y. (2013) S4153R is a gain-of-function mutation in the cardiac Ca(2+) release channel ryanodine receptor associated with catecholaminergic polymorphic ventricular tachycardia and paroxysmal atrial fibrillation. Can J Cardiol, 29, 993-996.
81. Marquez, M. F., Totomoch-Serra, A., Rueda, A., Avelino-Cruz, J. E. and Gallegos-Cortez, A. (2019) Basic and Clinical Insights in Catecholaminergic (Familial) Polymorphic Ventricular Tachycardia. Rev Invest Clin, 71, 226-236.
82. Marks, A. R. (2023) Targeting ryanodine receptors to treat human diseases. J Clin Invest, 133.
83. Santonastasi, M. and Wehrens, X. H. (2007) Ryanodine receptors as pharmacological targets for heart disease. Acta Pharmacol Sin, 28, 937-944.
84. McCauley, M. D. and Wehrens, X. H. (2011) Targeting ryanodine receptors for anti-arrhythmic therapy. Acta Pharmacol Sin, 32, 749-757.
85. Louch, W. E., Koivumaki, J. T. and Tavi, P. (2015) Calcium signalling in developing cardiomyocytes: implications for model systems and disease. J Physiol, 593, 1047-1063.
86. Tunwell, R. E., Wickenden, C., Bertrand, B. M., Shevchenko, V. I., Walsh, M. B., Allen, P. D. and Lai, F. A. (1996) The human cardiac muscle ryanodine receptor-calcium release channel: identification, primary structure and topological analysis. Biochem J, 318 (Pt 2), 477-487.
87. George, C. H., Rogers, S. A., Bertrand, B. M., Tunwell, R. E., Thomas, N. L., Steele, D. S., Cox, E. V., Pepper, C., Hazeel, C. J., Claycomb, W. C. et al. (2007) Alternative splicing of ryanodine receptors modulates cardiomyocyte Ca2+ signaling and susceptibility to apoptosis. Circ Res, 100, 874-883.
88. Wei, C., Qiu, J., Zhou, Y., Xue, Y., Hu, J., Ouyang, K., Banerjee, I., Zhang, C., Chen, B., Li, H. et al. (2015) Repression of the Central Splicing Regulator RBFox2 Is Functionally Linked to Pressure Overload-Induced Heart Failure. Cell Rep, 10, 1521-1533.
89. Homsy, J., Zaidi, S., Shen, Y., Ware, J. S., Samocha, K. E., Karczewski, K. J., DePalma, S. R., McKean, D., Wakimoto, H., Gorham, J. et al. (2015) De novo mutations in congenital heart disease with neurodevelopmental and other congenital anomalies. Science, 350, 1262-1266.
90. Glessner, J. T., Bick, A. G., Ito, K., Homsy, J., Rodriguez-Murillo, L., Fromer, M., Mazaika, E., Vardarajan, B., Italia, M., Leipzig, J. et al. (2014) Increased frequency of de novo copy number variants in congenital heart disease by integrative analysis of single nucleotide polymorphism array and exome sequence data. Circ Res, 115, 884-896.
91. McKean, D. M., Homsy, J., Wakimoto, H., Patel, N., Gorham, J., DePalma, S. R., Ware, J. S., Zaidi, S., Ma, W., Patel, N. et al. (2016) Loss of RNA expression and allele-specific expression associated with congenital heart disease. Nat Commun, 7, 12824.
92. Morris, S. A., Ethen, M. K., Penny, D. J., Canfield, M. A., Minard, C. G., Fixler, D. E. and Nembhard, W. N. (2014) Prenatal diagnosis, birth location, surgical center, and neonatal mortality in infants with hypoplastic left heart syndrome. Circulation, 129, 285-292.
93. Mendell, J. R., Rodino-Klapac, L. R., Sahenk, Z., Roush, K., Bird, L., Lowes, L. P., Alfano, L., Gomez, A. M., Lewis, S., Kota, J. et al. (2013) Eteplirsen for the treatment of Duchenne muscular dystrophy. Ann Neurol, 74, 637-647.
94. Hua, Y., Sahashi, K., Rigo, F., Hung, G., Horev, G., Bennett, C. F. and Krainer, A. R. (2011) Peripheral SMN restoration is essential for long-term rescue of a severe spinal muscular atrophy mouse model. Nature, 478, 123-126.

Informal Sequence Listing

| SEQ | | |
|---|---|---|
| ID | | |
| NO: | Description | Sequence |
| 1 | Mouse | ACCTGCTGTGAAAAGAAAAAGCCGA |
| | microexon 4 | |
| | SSO | |
| 2 | Human SSO- | ATATCAATTTGGTCCTTGCACCACA |
| | intron 3a | |
| 3 | Human SSO- | ACCTAGCAATGTTTCAATTACTGTC |
| | intron 3b | |
| 4 | Human E4SSO- | ATACATGGAAACACATACCCATTTT |
| | 01 | |
| 5 | Human E4SSO- | CAAATACATGGAAACACATACCCATTTT |
| | 02 | |
| 6 | Human E4SSO- | TGCAAATACATGGAAACACATACCCATTTT |
| | 03 | |
| 7 | Human E4SSO- | ATACATGGAAACACATACCCATTTTTCC |
| | 04 | |
| 8 | Human E4SSO- | ATACATGGAAACACATACCCATTTTTCCAC |
| | 05 | |
| 9 | Human E4SSO- | AACTTGCTGTGGAAAGAAAAAGCCA |
| | 06 | |
| 10 | Human E4SSO- | ATCAACTTGCTGTGGAAAGAAAAAGCCA |
| | 07 | |
| 11 | Human E4SSO- | ACATCAACTTGCTGTGGAAAGAAAAAGCCA |
| | 08 | |
| 12 | Human E4SSO- | AACTTGCTGTGGAAAGAAAAAGCCAAAA |
| | 09 | |
| 13 | Human E4SSO- | AACTTGCTGTGGAAAGAAAAAGCCAAAATA |
| | 10 | |
| 14 | Human SSO- | ACTTTTAGATCACAATTTCTTTGCA |
| | intron 4a | |
| 15 | Human SSO- | GATTAGAGGCGAGAGCAAGCATGCA |
| | intron 4b | |
| 16 | Human SSO- | ATAGTACAAGCCACACTAGGAAGCA |
| | intron 4c | |
| 17 | Human SSO- | TGTTCATTACAACGAAGCAGTTAGT |
| | intron 4d | |
| 18 | Human SSO- | ATTGTCTTCTTCTACAGTACATGCA |
| | intron 4e | |
| 19 | Human RyR2 | gcaagtacccaatttatgtagacttgtagtattttaatgagctcagctactataggaacaatttctttc |
| | Upstream | acaggtgtcagagattttctttttgctaaaataagtccatgcctttcagttggaatgtccttaggtttg |
| | Intron | actttctgctttatctagcatatttgtggtgtcttgaaaactaaaaaaatatatccaagcttgatgtga |
| | of Exon 4 | tctgtgtcactaggcctcgtgcttatgtctgttgtttaaaaccgacctttctttgagcgtttctgtgttt |
| | (Intron3-4) | cattacattttcagagctgttattgttattattttaactcatagctttatgcatggctatttgtcattaacc |
| | (microexon 4 | tttatttcattttcaaatgtctgtcactatgtgactatgttctgagctttttcatggaaagtaaaatttcat |
| | capitalized) | ggcactgttatatattttaagtaaccaaattttggtgatatggctttaggctattgtaattttgcaaaca |
| | | gctaatccttctcctctgaaaattgaaatttttcttttcttttttttgagatggagtctggctctgtcacc |
| | | caggctggagtgcagtggcgcgatctcagctcactgcaagctctgcctcccgggttcatgccat |
| | | tctcctgcctcagcctcctgagtagctgggactacaggctcctgccaccaccacacctggctatt |
| | | ttttttttttttttctatttttagtagagacggggtttcaccatgttggccaggatggtcttgacctcctg |
| | | acctcgtgatctgcccaccttggcctcccaaagtgctgggattacaggcgtgagccaccgcgc |
| | | ccagccctgaaattgttatgtggtttcctaatgcaagtatttttctttcaaagcctatttggttattttatt |
| | | ttcaatgagacttcctgtgactgatatctgggctgttacccagtcttaaccaatttattaaagtttaga |
| | | attaagcaggaggattaatggtcaaattttttgtttccaggttttttaatttttattttttggtatggtaatt |
| | | actgcagtgtatggtttaataaagtgtagtatttccagagagaaaactgaaaattttattttgccatt |
| | | attgatctatatgttgctgttgttattaatcattactctggtctggataaataaagagattaaaaaatg |
| | | gtaatatataaatgattatccatagaaaggagtgatcagttataacgttattttcatcagccttgtagt |
| | | ttttaaaattttagtgtcatgcagcttatgactttagttgatttgtgcattaattctaaattaaatttcagc |
| | | agtgtactattaagtagggaagagaaactttagtgacttttgatattagactagaagcttattttaca |
| | | aaagcttagtattttagtttttcaggttcattttttgtttacaacaaagtttgaaattattaaaatatataa |
| | | agtttcttttaaaaatttctattctttcatgtaaaccttctcagttatgcttaagttatgaaagagtaaaa |
| | | caattctcaatttagtttcatttctacctgaaataacccctcctgatttttaaacatccctgagtcatat |
| | | caactttgatatgcaaatcagcttttataaagaaagatcatttttatttggcattagcatttaaaaaat |
| | | gcatcttgccatttcatctgtctttatagataacaacttagaactaaattgccataaatttaatttttttat |
| | | tgttaagttgcgttttttaaatttaacaactttgagaagatcttttatcttcttcaaatcatgaggttggg |
| | | ctgggcaaattctaaagtttcttgaaatataggaagtaatttgaaatactgttttaggatttatggtttt |
| | | tttgagatggggtcttgctatatggcccaggctggtcttgagctcctgagctcaaacaatcctcct |
| | | gcctcagtctctggaatatctaggattacagatgcatgccacagtgtacccagcttctaggatttat |
| | | gttttcaagacatttattagttagaatttctggatctcctcaagtatttttatttagctgaatttagttttg |
| | | aattgttcactctctagaatgtctccatcacagataaagtagtaatttgtgtgatgtgttgaacactat |
| | | tttataaagcgcagtatagttaccataagacatgaattaggaagtataatttgaggaaaaactgct |
| | | acaaggaataaacacattgcatgaagctgaaacctatttctggttattagtaacttttagaaagaaa |
| | | ccaatgtcaaatagctcaacggggtaccgtatattagggaaaaatgaaagtttctctcatactgct |
| | | gagtatttttgattatacccagaaatgatattcattcctagcttgccacaggtgatgccagttaatga |
| | | gcacagccccagccacagcgttggaggaggagttctggaggcctctattgtatttatggatttg |
| | | gaggtagcccgagcaacggggggtgtcctggaggacccctggggtggtcagggttaggga |
| | | gcactgtggggacttgcggcagcaactgtttaccaagaaggatagatattttccacttaaccattt |
| | | tattcataaggatgtgcaccagctagatatcatgtctgatgatatgtgactcatggatgctgctgg |
| | | cacttgtaggaaccaagtcttctgaactccaaatatcatcttttctagtgccttataatgtccccttta |
| | | cactcattatggaactctaatggggaatttttttatcaatcaaattactatctccaggttaataaatcat |
| | | gtgtagtttgcttaaatgaagagccacttttcctaggatgaagtgtgtcaggaaggtaatgtgaatt |
| | | ttcaagaaatcatttctttcctttcagatgaacagtgagtcaaaaagaacctcgtgttgccctgattc |
| | | ccaactctttcataatcaactttctttacgaagccctgttcttttttatttaataattcttctttaatttctta |
| | | cgtaattcagtaaattaaacaatacttatactatttcatgtccaaaatgttatatgcacatagcaatac |
| | | aaacacacttgatgataaggaaaatttttccatttagaaccatgcctttgttttttgtggttaaggacc |
| | | attttgctagtattcttgggtcatgaataaatacatggttttcataaaatggttaaggaaacttagca |
| | | gaaaactcccaatgtcatttgtgacaaagtaaatcatgaacattacgataacagaagaagatgtg |
| | | taagatgaaatgggagaaaatattacctaatgtatgaagttttggttccctaaaaagttgcttattgt |
| | | gactattagtaaaataactcatatttttgtggcaccaaaatcattctgaaggatttcatctaagtgga |
| | | ttttctaaaatgtctgtttaattaaaatatatatatatatatatatgttaaaagagagacctagatccag |
| | | tatttatataattacattgactatcatttagcttttattcttattaaaatcatgtcaatagtaaataggcca |
| | | agacctgtctatctcagtagctgcccacagaagctttcagatcttgaggatggagacagtacaca |
| | | ggcaacgagaaggagaaggtgtctgttgagcagcaaccatctctttggccccgttttctctggg |
| | | cgtctgcctatgcacattctggcttctgctgttctttctatttcccccactgggttttctgtggttctgg |
| | | ataacttgtcgcttgcagatctacctctgtattgctcttcttgctcttcagatctattttgcgtatggat |
| | | gatatagcatagggttaaaaacaggggtttattgtcttagagaactgcttgagaataccagttctg |
| | | ccatagttagctctgtggctttgggaaagataactttatcatcaaggcttagtaattattaaatgatg |
| | | gtgcatgttattattgttttatttattcttttttgtatacttttaaactccctgggttggactgctattaattc |
| | | ctgactgccttttctctgggcttgatggctttcttccttctctgggacatagccagtccagttcccac |
| | | ttcctcccctcgaccccagtattctcatcaatcctggcacatgtagtttgccgatttaatgatgccg |
| | | cagcagtgttagaatagagagttgatgatagcatttgcttgtttccatgtgttttgtagttaataaagt |
| | | ttcataatttaagtaatattgtgcttttggctattccttttatcttaattcacaaaattatgatagacacttt |
| | | tctctcacacaactttatttgtaatgccaaagttaatttaaggatttcatctcctatgagcacagatat |
| | | caagtgagaggtgacgtagtatagtagaaagggctttggttttggagtccaggaaatttactgaa |
| | | agttctttttcaaataatatttataataattatcatctgttgaaaaggcgttgctttggtttaatgccatg |
| | | agtccaatatatggcttgtatattctgttgaggacagcgtattttaaaatacacgttgagtgtcctttc |
| | | tgtctttcgggtgaattatttctggggggattgtaggaccctgtccttttcaactttagcttatggcca |
| | | cttgactgggtccctttattattagaatattgtagattgaactagagaaaaaattaaaattaatcattc |
| | | tttgctccttacatcgtgctctttgatggagtaatagattaacatttataattcaggtgattatgatttg |
| | | gatcattcattcatttgctatataatcctcatgatcgtgggtattggtagtttaccgctatgtaagttat |
| | | ggatgatactttaaaaatctataaaattcacaatgtaatgttataaatattgtgatgagcaaataatat |
| | | tgaaatgaatattaaataaatccaattctaatcatttggtgctttttcaacatttataaggtaacttatat |
| | | cttataaaggttctatgtatagaaatttaagtgtttttttgtcatcaatgagtttacaactgcagggata |
| | | gaaaaacaattatagtataatttcatgtatgttatagaagtatatataattatcatgttggcatgaaga |
| | | aggaattgtgtacgtggaacttgttgttcctttacattttcttggcactttttagaatcatttccaaaac |
| | | tattagaagtaatattaaaactattaatagaaaaatcatttcaaaattttttccaaggaacaatttgag |
| | | ctcattaggcaattccttaaaattttaatagaaaataatacttgtcaggaagacaatccaaaacatg |
| | | tccagagatattcttaaaatgatttttatgtttcagtttagtaaaatggcttattgttttataagaaactg |
| | | taatgatgtttcctctttctaaattatgtactgtcttcaaagtacaaaatattacaatgacttaattatta |
| | | acagaggtatagatcagactcagacatgtccagcttttatgaaaaatggagctgtagaatgtgga |
| | | gtagtaaaaaacttgtgttggctgggcacggtggctcacgcctgtaatcccagcactttgggag |
| | | gccgaggtgggtggatcacttgaggtcaggagtttgagaccagcctggccaatgtggagaaa |
| | | ccccgtctcttatgaaaaatacaaaaattagctgggtgtggtggcgcacacctgtaatcccagct |
| | | actcgggaggctgaagcaagagaatcgcttgaacccaggagatggaggttgtagagagcag |
| | | agattgcaccactgttctccagcctgggcaacaaagtgagactccatctctaaataaataaataa |
| | | attaattaattaattaaataaaacacttgtgtcattctttttataaaacccctatctctttaagacatgta |
| | | ctctaagaacattcaaaaatgcatggggctgggcacggtggctcacacctgtcatcccagcaat |
| | | ttgggaggctgaggcatgtggatcacttgaggtcaggaattcgagaccagcctagccaacatg |
| | | ttgaaaccccatctctactaaaaccacaaaaaattagccagacgtggtgacacgtgcctgtaatc |
| | | ccagctacttgggaggctgaggcacgagaatcacttgaacccgggaggcggattttgcagtg |
| | | agcccagatcgtgccactgcactccagcctgggcaacagagcgagtatccatctcaaaaaaaa |
| | | aaaaaaaaaaatgcagtgggaagcaactggctaaatttactggtaaataaattaaaaaaatacat |
| | | ttgtgaagcactgaagccccataaattactttttaaaagtacatttatcaaacactgaagctccaatt |
| | | tcacacaagtaattgcatctaagatcattttatataaatgcaagagcaaaagatgaagaagatctg |
| | | tgtattatctcatagtggttaaagctcatagattatagtacaaactccctgagttcatttcctggtaac |
| | | ataagtgacttaacctttctgtgcctcaattttctcatttataatgtggggaaaataatagcatctccc |
| | | tttgttgtgggaatcaaataatataagtaaagtgtgtagaacaatcgtggcatgcaagaagcacta |
| | | tgagtatgctgtcatcattgtgatggtctctgcattgagtatgcctgagtaagctcctatcagatctg |
| | | atccttctgaagaaaataactataatctctggacaaaatacaaaaacaaaactacacccacaata |
| | | ataaaaccttcaattacctgaagcccctagctatagttgctctatggccgcctttacaagttaccgc |
| | | taagttggtggcttaaaacaacatacatttattattttctagcgttagaggtcacaagtctaaaatca |
| | | atctccattggctaaaatcagggtgttggcagcactgcattgcttccagggactctagggaaaaa |
| | | tctgtttccttgccttttctagcctgcattcttggcttgtgacccatcctccaccctcaaagccagca |
| | | gtgacctaccatgtcacatcgtcacaggtgccagggattaggatgtggatggttccggtgtgcc |
| | | attatgtgggctaccctatcatggatgtgctgagctcgagtaagatgttattgacaagatcaaatg |
| | | gaagtcagaacaatctagttgaaaaatgactgaaaagtctactttttgatttgacaggagcctgatt |
| | | ttcttgcaaaccgatcaataggaatttcagtctgtcttatgtacataccaatttatagattcacataca |
| | | ttttgtatttaacgggcttcattctctgaaggttttctgagtttccagtagttaatcacatgatttagaat |
| | | ttaaatttaagtatattttcatattgcatgcatagtagacactaattacttttaacaataacattaatcaa |
| | | aaataactaaaaaaatgaggatatatataaatgcaatttgttgagttcctatcgtattccagacagg |
| | | catcttcctaagccatgaatacaaaatagtgagtgtggtattatttttctttgttaatagcagcctgtt |
| | | cagtctttggctatagtctcttctgtgtcaggtaacagtatttgaagtatggtcacagttccaacagt |
| | | agatcttcatatttcttttacatcctcattcttctggtattatggtatctgctcagaggcactacatcgt |
| | | aaagccttttttgttcaagctttagggcagatagatcaaggatcaaaatgaaaagagttaatgttgt |
| | | gtccacgctagttgagttttatattaatgatttctttccattaaagcacattggggtaatgtggagga |
| | | aaaggagttgatgaattttgtagttcatatgatataaattgtgttctgtatattattaaaagtaaatttct |
| | | caaatggcagggctaatgactcactggaaaaaaatgaggtttaacacttgatgttcatccattttc |
| | | aaatatgctctgtatgagtctccattaagtctctgtataagtcttctgtattttaattctgtaaaataca |
| | | gaattaaactccattaaaatacagaattagtatgtaaaactgtgaccatactttaaatagtgtaaatg |
| | | tagagatacctgccactgaagagactatagccaaagaatgaacaggctcctattaacaaagaa |
| | | aaataataccacactcatcatgttttatttgtgtttcaggaagatgcctgcttggaatatgataagaa |
| | | ctcaacaaattgagcatttatatatatcttcatttgtttatttagttatttttaattaatgctattgttaaaa |
| | | gtaattagtgtctattatgcatgcaatatgcaaatatacttaaatttaaattctaaatcattgaaatgttt |
| | | tatttttataacagtaatatatgagtataaaataaaaatgccctcttcatagtcctcattcccccctaa |
| | | gataaccacagctaaaagattagtatatagccatctataactttatacataaaaatattattatataa |
| | | atggaatcatatactgtcttgtcatcttttaccacctaattatatatggtttttaaaaaatcaggacaca |
| | | aaatgtccagaggaagagaagattcagatttagttgatccaagctctaggtgagatttctgtagtt |
| | | ctctttgccctgttctgtgtttgcttggttcacaggctagcttccctcatgattacaggataccttgta |
| | | ggaatttcagggatgatatccaaaggcaacatcatctataggcacaaaaaggagctgactttctt |
| | | tatttctaaaagcaaggcactttccccaggaattccctggcagaatttccctcatacccattgtcac |
| | | acacccatagctaatttatttactcgcaagagaaaatggggccagcacaactggctccgaccat |
| | | ggggatggcaatagggaaggatactttgaagaactaggttcttttattgggagaaggaagaga |
| | | actgcatgtgaaaatgaaggtcagataatttctaacattgtctgctacatcatatagcagatacata |
| | | gttagcatctgtatgctttcactggaaatgtttatatttgggaccctccatgttcttgagccaaacta |
| | | ggaccaacaagtaaaatctttgcctatgaaaacaaatccaactctgtgctcaggtgtctccatga |
| | | ccaagaaatagaaagcgcatgtccattctccacctgtctcattcgtacagtgttcattcaacaaaa |
| | | atagattgagttgcttttgtgcattctggagctagggggacaggtatgaataacaatgcctccttg |
| | | ctcttgagaatcttagcaatctaataataaagagagaagataaatataatgtggtatgaatctgcttt |
| | | catagtaaatagttcaatcagagagcagagaagagatctaattacctacccaacactctcccctt |
| | | gtacgtcctcataactgtgaaatgctgaatggattagtgtgtgtttatattgtagacaatacgatgct |
| | | actagctatttatatctgaaacttctattctcaaatatctagtctaacttaaccttattttaagaggctgt |
| | | gttctgttacctacctattgaagaacatccttggtaaagcataaatagtacttcaataattagattata |
| | | atgccaataaattcacaaatttactaattaatgtaatatttagccagcatcttgtccaaagcctctgta |
| | | tataaatatttacctacctgtcatctgcaaaattgatgcagtgatgtaatggtgaatgtgattataatc |
| | | tcttgaagggttcatagctaaaaatattcactgagcaacaattatccatctgtcacttaggtgaatgt |
| | | ggaaatttgtctgtggttgaaagatgcatctttcagttttactgcctttctgtttctagaaagacaact |
| | | ttctactcagaagccaaggatattccatatttctaagtatttgaatgacgtggtttccagactctgtc |
| | | atcaaactcaataaagacgtcaatttaagcaggatgtaatttaatcacagtggcgtagttaggcac |
| | | tgttttagattatgaatcttaaatgtatagattattttatttgattagtctcattcaatttggacatcttcct |
| | | cagctatttgtgtgtgtttataatagaaaaaattttcatttgtaaacatatgatagcaaataaatgaca |
| | | tggcatatttctgattcaatcttaataaactattctgcaatttatgagtgacttaaagatatgtggaac |
| | | ctattgagtcagccattctcaaacatttttcttttttgagatagagtctcattctgtcacccaggctgg |
| | | agtgcaatggtgcaatatcctccccaaaaaacaaacacaaaagcaaaaacaacaattaacact |
| | | ccttcctgcatttttttggtgtgttttcttcaaagcactgaacaccttctgaagactatgtattcgcggt |
| | | ctgtttattgccttcctatccccattagagtgtaaatctcaggaatgcaggtctttgggtctgtggta |
| | | ttcgcttgctataccactaacatccaagacaatgcatgttatgtactaggtagtaaggaaatatttg |
| | | ctgactggagggactgcaagatggaatttatagaccaaaatcaaggggctgaaaagtaccaaa |
| | | aattctgtttctgctactgtagtaacagttttgaacatatgatagagcttttttgtctgttaaatacagc |
| | | tgtttttgaattgcctctctattgtgggagaaaaggaatgtttttgaaaggaaaagtcagaaaataa |
| | | ccttttgggttcggtaatcctccatcctcatttgcctgacacagtccaggttaatacctgtggtcac |
| | | agcataatcactattagttccccctttcgttgtccaaagtgtcccagtctggatgagaaattacatg |
| | | gttatccctagtcatggtgtctttgtgaatcagataaaacttatagaatctatttacagaaggcaata |
| | | caaataatatgcacgcatacaaaatcttgtatttcatctctgatttgaagacttaaaagaagtcttcc |
| | | tttttgttgagttaaataacttccttttttttttttgagttaattcttgctctgttatccatgctggagtgca |
| | | gtggtatgatcatagctctctgcagcctccaatgctgggtctcaagggatcttcctgcctcctgag |
| | | tagccaggaccacaggcacaagccaccatgcctggctaattaattttttttttttttttttttggtaaa |
| | | gataggttcttggtatattgcccaggctgtccttgaactcctgaccttaagtgatcctcccacctta |
| | | acctcccaaaatgttgggattacaggtgtgagccacctcaccccactcatacaacctctttagtg |
| | | attgttgcagtcacacattctgagaagatacacttttttgcaagtaatccctgaagtgtgagagaa |
| | | gggcaaagaccagcaggatctacataacttgaggattacagagaccagatggaacattctgag |
| | | agtgagcatgaggtcagtactcgtagtacaaactgattactttcatgtggcttctgagatcaggcc |
| | | acatgtgacccatgacttcctggtgaagcattaagtcttaagaccacgtggaactgtagaagggt |
| | | cctttgctggaaccagaaaccttagaactgtccttagaccactgaaaaaagtacaaagagcagc |
| | | gcagcaagaacacacagaatgggcctaatattgccgcgcactgatttccatagccctggaaaat |
| | | gtgagccatatgtgagacaagaagaacatattgaaagccttgcagtatctgacaaaagagaaa |
| | | agtaggccaggcgctgtggcccacgcctgtaatgcaagcactttgggagtctgaggtgggag |
| | | gatcacttgagcccaggagtttgagaccagcttgggcaacgtggtgacactgtctctacaaaag |
| | | ataaaaaaaattagctggacatggtggctaatagccatagccagcatacctatagacccagcta |
| | | cttcagaggctgaggtggtgggattgcttgagcttgggagatcgaggctgcggtgagctgtgtt |
| | | tgtgccactacactccaacctgggtgacagagcgagatgctgtctcaaaaaaaaaaagcatcc |
| | | caaatctcacacaacaatgaatgagattggacaatgcaaagataaagctaaatgaaagttaaat |
| | | aacacgagagatcacagaagaaacagcagctaaaccagcaaaattaaatacttcataagctgg |
| | | tgttaattgcatttgttcactctattgatatatgatgagtaaccacgttctagattctgtgtagagtaaa |
| | | tagtgagagcgagctgaattgctacagaggcctcttccagaataggacataaatggagcgtgt |
| | | gcattgtagtgtataaacactcaataagtgattttttttttcagatgaagaaatagaaacaatgtacc |
| | | aaggtcagcgattaaaaatagtgattatctttacacagtcatggactggaagttttgtttctaatagt |
| | | ttccagaatagaagtatttgtgtgaatgacaattatatgacatgtaaggaaattaattatatcatttat |
| | | tgattcttgaggtacaggctctaaagctccagcatggatttgatgtggatggtactaatgatacag |
| | | atatttaagttttgttgatttgtatgtagtttccccataagaggttttttgttttttttttttttttgagacaga |
| | | gtttacttctcattgctcaggctggagtgctatggcatggtcttggctcactgcaacctctgcctcc |
| | | tggtttaaagcgattctcctgcctcagcctcccaagtagctgggattgcaggcacccaccacca |
| | | cgcctggctaatttttgtgtatttttagtacagatggggtttctccatgttggccaggctggtctcga |
| | | actccttacctcaggtaatccgccggccttggcctcccaaagtgctgggattacaggcgtgagc |
| | | caccacgcctggcccccataaggggtttttagttcctcgggaaactattcaagcattggttagga |
| | | agaaatatgtgcagctgggtgaatccagctaatgatcggattattctcatggcgttttgccgggg |
| | | gctgccttgatgatgtcttcttggatctaggtctttaacttgtctgtaactattgcacgctgtccctcc |
| | | agcagaccgttcctaatttaactgctatcctgtgacctcagttcttacaaaggaatctaaaactgta |
| | | acctttctttatttctggctccaattagttctttatactattcgttctctatccaaactgaactcactccct |
| | | gaatatgctacacttaattcaggtatatattctccacattcttactacactaattcctcaacctagaatt |
| | | ctgagcctttcaaaagtttaccacccctcgcttctctgcaaatgtccaaggagttgtcttccttaaa |
| | | cactttcatgaagctcccagccagaagtaatatttctactcgtagagttccttatcactgggtgttca |
| | | cattttcttatttttgtctctcattttatatagtgattatttggtatgtattttatacattttatttacattagaa |
| | | tatggttttgttcaatgtccattctttaaccaacaccacattgtcttgattactagtagctttatagtaa |
| | | gtcttttttatttatttatttattttttgagacagagtctcgctctgtcactgggctggagtgcagtggc |
| | | accatcttggttcactgcaaccaacctctgcctcccggattcaagcagttctcctgcctcagcctc |
| | | ctgagtagctgggactacaggtgcgcaccaccacacccagctaatttttgtatttttagtagagac |
| | | ggggtttcaccttgttggccaggatggaatccatctcccgacctcgtgatccgcccacttcggac |
| | | tcccaaagtgctgggattacatgtgtgagccactgcgcccagcctgtagtaagttttgaagttac |
| | | ataatgtctgtctctgactttgttcttctccaatgttgtgattattgtgggtcttttgccttcccatataaa |
| | | ttttaaaatcagtttgtaaaaatctacaaaataacttgctgagattttgattgggatgcactgacttgt |
| | | tagaatgtgtaaaaatttttctgtattcctctgtgtacctgccagtttttcatagtatatagatctaaaat |
| | | gtaagactttattatatgaaatttattattcataatagatctaagcccaggaggtcaaggctgaagt |
| | | gagtgctactatattccagcctgggtgaaagagagagactctgccaaaaaataaaaaataaaaa |
| | | ataaaaaataaaaaaaaatatatatatgtactatgtacatatcctgaggatttgaacaaattcagaa |
| | | aacactgaaacagtattacatgagctggaatttatttaagagtagtaatcaacttgatttttttctatta |
| | | attttagctaagcaaaagttccatcattattcatgactgatgatttctttccttatatttattgtgatgact |
| | | ttttaatcatacatttaaactaatacacattcctagaggtggtactctaagctgaatattttctaacaa |
| | | agtctatctattattatgtctaataccacatttatcactgtattttagaaatgtttaatattaagattagtc |
| | | cccctcattcttttcccctgacccccaaattgttcttattcttcttgactatttatctcccaaatgaaatt |
| | | acttgtggtatttaatttaacttagtaattctgataagtagcctccatggaagagacttactgtattatt |
| | | tacttagcacccatccatccacttgaatttgttaacattaaggtttatgcaacagccactttaacgta |
| | | ttctgtacttagagaaaggagatattgataattttgagttagtgcatatccaggctgggtgcggtg |
| | | gctcatacctataatcccagtactttgggaggctgaggtgggcagatcacttgagcccaggagt |
| | | tcgagaccagcctgggcaacatggcgagactctgtctctacaaaaaaataaaataattagccag |
| | | gtgtggtgcaacgtgcctgtagtcccagctacctggaaggctgagaggtgggaagattgcttg |
| | | agcccgggaggtcaaagctaaagtgagtgctactactgccactatactccggcctgggcaaag |
| | | tgagagggtctacctcaaaaaaaaaaaaaaaaagtgcatatcctgaggatttgaacaaattcag |
| | | aaaacattgaaatagtattacatgaactgttcctattctaaagtaagattaatttaggaagtagtagc |
| | | tcttagttctttattacctagtttaaattttaaaagaaagcatttatacttaagaataaaatttctaaacc |
| | | ctttccttgtattgagttgtatgtgtgttagtgtatgagattgctgagtttattgtactgagtcggcttc |
| | | attgtatgacagatctaaaaaaactgggagagttctgcctaatactgcaaaaccagagtaaagtt |
| | | aggaaattgggctaatgtttaattaagtcaatcactggccttacttttgtgcttcatttgctcaattac |
| | | acttatggtaaatttctttctcgaactgatggagaaaagactttgtgatctcatgccgttaaagtaca |
| | | taatcccatatgatattacagtatggagagatgcttttctttttatttataattctcccacattaatgtca |
| | | tgtctgtctgttctaatttagtgctttgtatttttcattttaaaagtacctggtctgctcctgtgaacattt |
| | | aggtagatacctcagtttgaggtcctgtgagtctgtgattctattatttgtaaacatgtatgaaaatg |
| | | gaaaagaggttgggcatggtggctcacacctataatcctagcactttgggaggcaggaggatt |
| | | gcttgaagctgagagtttgagagcagcctgggcaacaaagagagacccctgtctctacaaaaa |
| | | atttaaaaaactagctggacgtggtggtgcgcacctgtagtcccagctactcaggaggctgag |
| | | gtgggaggatcactagaacctgggagttccaggctgcactgagctatgattgtaccaccctgg |
| | | gcaccaaagtgagaccccgtctcaaaaacaaacaaacagaaaaaacagagagggaaaaata |
| | | aagaaaaatacatatgtataataataggttctcaatatatattagtttctttccctttctactttcgggat |
| | | gaattcactgcttgttgaaatgtaattgactgatctaaatgctgatgttttaatgaaaagtatataatg |
| | | tggatttcatttaattctgtcctaaatgacagtttgacgacatacttaacaaatccctaggcaccaa |
| | | ggatttggggttttgttgggaagaaaacatctaggccagaaacgatgtacttttaccttgttttgaa |
| | | tcttttattgtttggatcagacctcgtttaaaaatgtagaggaaaaatttaatagttccatttctacctg |
| | | gaaatactacctagaaatagtattagtctattagtagactaatactccagctgtgggaaaaacaaa |
| | | aaacaagcaaaacacccaaacctcccccaaaccagaagttttaaaaggattaaatttaaaggat |
| | | ttaatcagaatatattaaatttaaaaggaaatatgattgaggcattagaaacttctgaggtagcgag |
| | | gacttgaagagacctaaatcccagcagtaagagaagtgcactgaggtaggtctaacattttttat |
| | | accacttttccctttggggcatttgctgagtattcatttgggcagagattaaggcagaggagcctg |
| | | caaagtttatgtaatctctggcctgacaacgtaggcagaatttgaggatacaagatcttgaagga |
| | | aaaagagaaataaggcttacactctgaagaggctttccccttgaggtatttctctgttggtggtga |
| | | cgtttttattattattattacgagacagggtctcattgtccaggctggattcgaactcctgggctcaa |
| | | gcaatcctctctcaagtagcttggataactggcatgcaccactattccagatcatttttctgttatta |
| | | gcctttgggaaacaagctcagaagcttagcagaaagcagatcaaaagctcagcagtatagtaa |
| | | gatgtgactatcgattctatgaaacaatactaataatgtctcatgaggcctaatcttgtgctggggg |
| | | gccaaaaattgtatttcagtggttgctaaggaggaggggttttgataatcattccaggctctcagtt |
| | | ggaatccttggaagttatgattactaggagagagggtgaactaaagtcttgctaagtctgaaaac |
| | | ctgccttcaatctactcagctcacattgaattggtgttatctgcccatcatctagctgccagccgta |
| | | ggtacatcttttctggaggaagaaacagaattagcttgtatacaaattttaacacagtatctgatgt |
| | | gcagtcaaacgtgttattaggcatgtaaggagtcaggatcaagagaaaaaagatctgacagta |
| | | gaaatagacccataagtgatccagatattggaattatcagtcacagaccttaaaacaattgggga |
| | | atctacaatgaaaaaaatcaaatggaaattcgaacactgaaaaccacagtaagtaaagtaagaa |
| | | ctcagaagattagtttaacagcagctgggacatagttgaagaaaggattcatgaactgaaaaata |
| | | gattagttgaaaatatccagactggcctatgtggagaaaagaacattaagaataaagggaaaaa |
| | | atcataaaagatgcattaactgtagtaataaaatctaacatgcgagtaactggattcccaaactgt |
| | | gatggatttggagctacaatagtgagattcaagattactgtcaatcctaaggaatattaatacttat |
| | | aaaaccacactgggttaccatagtaaaaatgctgaaaactagaaaaataaaaaatcttaaaagta |
| | | actggaggagggaacaaaatttaaaagctgtaataagactgatacctgatgacacattagaaaa |
| | | agttgaatcagaagacaaaggcatgatatctttaatgttgtgaggaaaaataatcactaactctgt |
| | | attatagccagataaaagtatacttcaaaagtgagagtgaagtaaagacatttccagacgcatac |
| | | aaattgagataatttagatacaggagatggggttcttcaggcagaaaggaaatgataccagact |
| | | gaaaagagtaaatgcaggagggaaagaagactaagtaaatatatggccaaatgtaaattaatg |
| | | ctaactatacaaaataatcgtattaatgccttgtggggtttaaaatatatgtgcaattaaaatgcata |
| | | acgataataactcaaaaggtggaagagtataaaattgtcaaccatgttcaaagatcctttttctgg |
| | | aacaaggtgaagttactaatttatacttataatgaaagagttaatcacaatggaagattattaaatgt |
| | | catggatgcccatgcaacctctagggtactaaccaaaagaataagaaagaataaccaacaactt |
| | | tccagagggaaaaatgcaatatcaagatatttgattaatgtaaaggaaggaagaaaggaggga |
| | | aaagaagtaacatatgggctgaatctgttcttaaattagattatggtgatggttcatagctgtgtaa |
| | | ctataccaaaaagcattgaaattgtgtaccttaaataaatgaactttatgatatgtaaattatatttcc |
| | | ataaagctatataaagagaacatgagaccaatataatacaaataatatgaaaatatgataaatata |
| | | actaataaatataaactcatattttaagaataaaactaaatgttaattgactaaatacttcaatcaaaa |
| | | ttcatggattgtcagattttattaactgcaattgtatgctgctcaccagagatatactttaagactgtc |
| | | ttacagaaagagtaaaagctataggatggtaaagttgtatgtgtggtaaagttgaatctaaagaa |
| | | agctggtataactatgttaatatctgagaaaagggctgggcgctgtggctcatgcctgtaatccc |
| | | agtactttgggaggtgagtggatcccttgaggtcaggagttcaagactagcctgaccaacgtgg |
| | | ggaaactctgtctctgctaaaaatacaaaaaattagccaagcatggtggtgtgtgcctgcaatcc |
| | | cagctactcgggaggctgaggcaggagaatagcttcaatccaggaggcagaggttgcagtga |
| | | gctgagatcgcaccactgtactccagcctgggtgacaaaagtaagactctgtctcaaaaaaaaa |
| | | aaaaaaaaaaaaaaaaaaaaaaatatatatatatatatatatatatatatatatctctgacctctgag |
| | | aaagaatttagggcagaaagtattgcgagaaatcaagatggacatttccctatgttaaaaaaagt |
| | | cagtttataagaaaaataaaataattctgaatttacatgaatctaatgatatagctttcaagcttataa |
| | | aaacaaaaattgacaaaattatcaggagatctcaatcaacaatcaatgtataagaccatccttctc |
| | | acagtattacaataatcaggcaaataaatgaatgaggatatagaaatctgaacagtatgattaatt |
| | | aacatatgtattgacatttatagaacacagcaccatatgctgagaaagtacacatttgtttcaagtg |
| | | cagatggagcatttaccaaaattgattatggaggaagtataagcaaatttcaaaggtttgaaataa |
| | | gagtgtattcctttctcatcacataattcagtaagaaataagtaacaaaaggaattgcaaatccata |
| | | agcctatgaatcaacgagttaatcaaaatggcaaattataagatattttaaattggataatagtgga |
| | | aaacatggatacagttaaaactgtggatacagttaaaactgattagaggaagttattcggcccta |
| | | aatacataagaagaactgaataatcagttatctaaatatctcaagaagacaaaaagaaaatattaa |
| | | acctggaaaatgtagaaagaagaaattatgaagtagaatttatgtagaaagcagaatttatgaag |
| | | tagaaagaaatataaatcattcaagccaatagctggatttttgaaatgactaattgatgagcccttg |
| | | gaaagactgaatacgaaaaagagagaatataaaattaacaatattagaaatgaaaataggaaat |
| | | cactgcagaacctccaaacaatgaaaagataataagagaatactgtgactagctttgttccaaag |
| | | aaattaaaacattctagaaacaatatagtgttagcgaaatacagtttaccaaccctaacactagaa |
| | | gtgggaaatatcaactgtcttatatttattaaatgaaatgaatttataattaaagccttctcacaaaga |
| | | agtccaggcccagatggcctcactggttaattccaccaaacatataaggaaacaataataccaa |
| | | tcttaaactctttcataatacttcccaactgattttatgaggccaacataaccctgatcctaaactatg |
| | | acaaagaccatgcaaaaaaaaaaaaaaaaaaaaaaaaggtatagactaatatttatcagggact |
| | | gtaggtgtagaaatcctccacaaaatattaaaatattgggaaatccatcccattgacatatcaact |
| | | caatagcctattacaaccaagttgggattcaaaaccaattattgtcacttaccttattaagataaaa |
| | | aaatatttattttaacagatactataaaacatttttaaatattaaacacctattcattgtaatattagtaa |
| | | gtcggcatgtaagaaaagttacttaaactgatagaaattatctatcaaatgcaaaactggtgaaat |
| | | attgaattctttcaaggcagtgttgaataaagttggggaaagtggatacttttggaattaaaaatga |
| | | gcttggcaaagtcacagctcactataaaaattcagttatatcagttatatttttatattctagtaaaaa |
| | | gcaattacaaaataaaaataagtgatttcacttatattagtataatgaaacaacacatacctaggaa |
| | | taactctaatacaagatgtgtaaacctttacacagaaaattgagaaacattttgagaaaaataaag |
| | | atgaccttaaaaaaaggataaatataccatgttcatatattgggaggctcaatattacaaagatga |
| | | cagttctcttccaattgatcaggtagatcaatgcaatctcaatcaaaatcccagcaggttttctttta |
| | | gtgtgcatgaaagttggaaaagccagttctaaatgaagaagagcaagattatcaaatacaacttt |
| | | gaagaaaagaacaaggttagagtctttgactcctggatatcaagacttattttaatgttacaataag |
| | | accgttgggtattaggacaaagacagaaaaatagagcaggatagagttcataagtagatctgta |
| | | catttgtgtcttgatgcattagtaagtgacaatgcagtgctaatggggaaaagatttattttttaataa |
| | | atggtcatttggataggtatacaaggggggtgaggagacaactcttaactcccccctaccatct |
| | | tacataaaaaatcaataccaggtatgtcgcaactttcatgggaaaggcaaaataatgaactttcta |
| | | gaaaatttagtagctttagtagcttcgtgtcattttattttgttgttgaagatttgcgtgtgatacatttat |
| | | atgtcatttgttttctagtatgtaaaatgcagataataagattacatgtaaaatgtatggctcaaagc |
| | | ctgccacatggtaactgctttaacttaacagaattatccatcttaaacagtaaaataaagttaatca |
| | | atttgaaagaagcaagcaaactttttttaaaaaagtttatggaatagttcatgtttatgtgccctaaat |
| | | ttgtaattaaaatatgaatgcacgagatgggtggatcacttgaggtcaggagttcgagatcagcc |
| | | tggccaacatggtgaaacaccatctctactaaaaatacaaaaattagccgggtgtggtggcggg |
| | | cacctgtaatcccagctatccaggaaactgagacaggagaatcgcttgaacccaggaggcgg |
| | | aggttacagtgagccgagatttcaccactgcattccagcctgggtgacagagtgagactccgtc |
| | | tcaagaaaaaaaaaaaaaaaaaaaagaatgcaaaaatagagatgtcatttgaattatctgacttt |
| | | catatagtgatataatttaaaaataattacttttatattagattcttatgtcctctctgctaaatcaagttg |
| | | gtgtttaaataactgttgtttcttttaagggaaattttgtttttattttgaaattttgaaatgaaattgctct |
| | | ttttattagtgttaatatgatagatatttaatgtaatttcctgcagatattaaaatagtcattttctttaata |
| | | aaatccacatatatttttaaaattccacatacttgattcaagtttttggaaaaaatgttcctttgtcttat |
| | | atgttttatgggtttgtaattgcgtaaattctaaaattggttaagtttactgtgaattttatatttccttgtc |
| | | ttgattagctccatttgaatcattctccttttcaccgtttgcaggaatttaaatgattcttgtcaacaag |
| | | aacaagagacgtgtcatgaaatcgttctgcattgcttatgcataattacaactttattgtatgttagtc |
| | | agtcttttaaccctgtggaccatttgtcttgtcttgtgtgtctagctgataattcactcccagcaatgc |
| | | tgttgctaagcttttctttgggtatttggatattctgtttgttacattattgctcctaattcttgcgccttg |
| | | aagaactgtgtcttttgtaatatagaacattaaagagctcctgttactatcattagtaactatgactct |
| | | tgattataaattctttcttctgaaaaaatcccataactttatctttgcactgatgggagtgtgtgtgtgt |
| | | gtgtgtgtatgcatgtgtgaatgtctgacatgcagaagtgaacctgataatggcaaaatacaatgt |
| | | ctttgataatcgttgatagcactaaaaatatgcactaaatatgcactaaaatagcaccaaaaatatg |
| | | gcatattctattttcctgataaatattaaaaggaagtcatttaatttttttagatttttttggggaagaga |
| | | ataaatataaaaattcgcccttttagaggtagaaaagtgcacaattgcactgtgctggacatgcct |
| | | tttttgcttctgtttgccccaggatctaaaggaatggattcatcactccgataagttaatgtttaaaa |
| | | ggagatcttcagctttatctaacaagaaaatatcttgaaaaatgtatagtgaaaatcaggcctttct |
| | | gttcagtcaagtgtttttcttaaataacaacagtgtgtttgtaaaatgcaaaggaggaagtaagttt |
| | | agaacagatttttctgaaaactaagtaaattacaaaagaaggcaaaatgctctgttacaagtaacc |
| | | tattggatttcattagggcttcataaaattctgcctgtttatttaggctcatattttaaatatcactttata |
| | | acatttttgaaataatcactctgtatgttgctttttatgcatattgcttcccaaaagcttgcttgccaga |
| | | gcattggagattcctagctaatagcccaagatttctgcctaagtagcattagcttcttacctaattaa |
| | | tgagttttctaaggaaaaaaaattacaaataaactttcatacatgaagggtgtgaagcaagccac |
| | | aattcccacaggacaccaattatacattctatagatgggaggtgagttaaggaaacttacctttga |
| | | gattgagtatgtctctcaatgttgaaattttttattctgatttatttgcaaggaatcctagggcgttgta |
| | | gagacagtctttgttgacatatggcttttttgtttgttctgtttacagtgtgggtatgattatagtcactt |
| | | aagttttaaagagccacataatgattaagagactaacattcacttataaatacaactttaaaaaata |
| | | atttgattcccacataacttcagaactatttgttaaggcatgcctttatattgtgaaattgtatgaaat |
| | | gctagaaagattagagtaagagaaattttactttaagtgtcatgagagaaactgtagttagacact |
| | | atgtctctaaattgatctttttaattaaatcagtttttataattagaggagagtagtagttcttaaatcag |
| | | aaggcctattacgtttgagaattagagaaatgttcatgtttgcttttaaataaattggattatcattatt |
| | | ttatgtacctggtgcatttccagcctaatgtctttttgagttctgattgctaaattaactgaattctttttg |
| | | tttggtgagattaaatgttgttaatgaaaaacttgaatacaattttttatgctttagaattggaagtag |
| | | attgtggtgcaaggaccaaattgatatcaattcatttaaatgacagtaattgaaacattgctaggta |
| | | tttgtttgtttgttatttattttggctttttctttccacagCAAGTTGATGTGGAAAAA |
| | | TGGgtatgtgtttccatgtatttgcaaagaaattgtgatctaaaagtgcatgcttgctctcgcctc |
| | | taatctttcattttgcttcctagtgtggcttgtactattagtgttcaaactaactgcttcgttgtaatgaa |
| | | cacatgttttacaggtctctaatgagaaccttatgtttctggttttgcttcatctgaaatggggcatat |
| | | taaagtactttccatccctagaaccacatagtttttatataatttattttgtaattattatgtgcttctggc |
| | | caacagaaaggtgtttattattcatactgtctgtatatcttctgcaaaaaaaaacaaaaaacaaaaa |
| | | ccttgactgtttaatctcaaaattccactgcttagtccttatatttctgcatgtactgtagaagaagac |
| | | aattatatatatagtcatagaaagtcatagaacctttttttttttttggtatagatggagtctcactatgt |
| | | tgcccaggctggtcttgaactcctggactcaagtgatccttctgcctcagcctcccaaagtgctg |
| | | ggattacacacaggagccactatgcccagccttatataatctgaagcagtgaaggtgaatgacc |
| | | atgtagaaaaacgcagttacaggattctggaggctaattcttagagcccacattgtcacaagttgt |
| | | gcaaaaagttacattgcttcttttggtctcggtttccttatctgttaggaaaaaataaaggagagag |
| | | gaaatacaccaaggtaccttccaatacttaacacaccaggattctgaatggaaattaaagtctcct |
| | | caagactgatattcctactccactttctcccaagagtgcaaaacagtgaggcttttcattattgaaa |
| | | tgtttttgtatttgctctgtgacttatatgaagaatcctttaagattgacttaactgatggctttattttat |
| | | cttcattatttttctggttttaggatccaactttcttgatgcaatattttgagaattttgccatcttttcagt |
| | | gccctttcagtagtgttttccaacaatttcatgttctcttgtctttttttttaaattttttttagccctttaaa |
| | | gacaaggaaacttgttttttaatttaccatttctgatgacctttgaaaactatgacttgtatgttatattg |
| | | ctgagaataatttgaagtcttcatttctctttataaccgaactactactacttttgaccaactatttgtttt |
| | | tgaaatttcctaccttcctctttggctatagttaaaatgctgatccaagattcacatttttcttgtcttgg |
| | | taagggaagcctgcttccttctcagaacacgtatgacacctctttgtcctgcacttccctaatatca |
| | | gcatcctacaacatagccctctgtcttttcccatccttgcatcccttcacttccattacactatttctgt |
| | | gccatatctggattggtattgaccaaccactcaagctgcttttacttactttgtaaatagcacttgcc |
| | | agtaaataaaatcaccttttcaccattgtctaaaagtcttcccttcagttcctctgtggatttggttact |
| | | tttgatctgcctgtgtaatttttttctttcttattcctataataatttatttttatttgtattaagtttatttgtaa |
| | | atcattgcagtagagattgccttctagttctaaggaaatatctctgatattttctgagctaaaaacctt |
| | | gaggettagtttgccaagtgactggctttataatcttatgtagcctctttattggccgtaatctctttgt |
| | | ctgtaggactagaatctgctttactccttttttcttaagtattactatcatgtcagtgctaaaacatatt |
| | | gatgccaaaatacatgttcatttggaagtcatataataaaaagattctgctgggttttagaacacgg |
| | | atgatcactatgtgtaaatgttgcagcaaattttagattggagctctttttttctttgaagacctatcca |
| | | gttcatgtcttatgctttggccattagaagtttgaaattctactgttacaggtagactttgaaggcca |
| | | acagaaagtaatttaaccctctacccacctaacccaggactattgagactcctctgtggggctgg |
| | | tacttctctgccccttgagttaaattagaaccggtcccttgacctttctcgtattccaagagtgaaat |
| | | accccacagacatttcaagtcatacagatctcatttcagtaattaaaatgcccaaatctgtataata |
| | | ggtgaacttgaagctaattcaagttaacttgtcaggaatcgagctggagggacggatatctggg |
| | | aagctgtttcttgcttgccccgccgggttcctcacgccctacttggtacctggagagtctgccact |
| | | ctcaggtgctgtgggaaggagacagaagagaaagcttggcaggaaaggtctgctttgatcata |
| | | gtgtcatgcactgggcttgccaggtttctaatgctgactttctctgtgggcgcttggcattggtcttt |
| | | cggggagcttctatccccagaaaactctgacctgagcttctcttgctgcagacaaatgcgtgtcc |
| | | actagctggtcagtggcaattccttactcttcctgggagttcttggtggcctctgagacaatccca |
| | | agctggatctgtctagtatggcccccatttgcttgttggcagtgtggtcacttcccagttgcagcc |
| | | ctgtaggactacacccagccttttgcctctcaatctccaggcatagtaagacaccagtctacagt |
| | | gtcccagctcccagggatatcaggtatccctgaagctacccttgctaggcttgaagtgagggca |
| | | agcttgggaggcaggcaggattccaggcccacctgcacttctctccaagaaaccatttctttgct |
| | | gcctggcctctaataatacactggggggggcagtcagctaagggcagccagtggatttgcag |
| | | ccaccctctttgacatccctgcttaggtggcataacaatatgttttgggacgtgatagtacaggtg |
| | | ctcctccagttagggtgaggttatatcctgaaaagcccttcagaagttgaaaatatcgtaagcca |
| | | aaaatgcatgtaatacacctaacctacagaacattatagcttagcttagcctcccttaaacgggct |
| | | gagaacacttacattagcttacagtggggcaacatcatctaacacaaagcctattttatactaaag |
| | | tgttgaatatctcatgtaatttattgaatactatactgaaagtaaaaaatacaatgattgtatgggtaa |
| | | tcaaaatacagttcctgctgaatgcaaatcactttcacattgtcgtacagttgaaaaatcataaata |
| | | gaaccattgttaagtcagggaccttcttgtaaatccccttcggagactggtcattgttttgttttggg |
| | | acagtgacctgttttaacttctgttatacttgcttgtctagggtgcgtttaccaccaccagattaaga |
| | | cctttgggagccttacaaacttaaatatatatgaaaattaaaatggcttctgtataaatcttctgataa |
| | | cattattctggatagtttcatcaaaggtccatcaatcactttctctttctttctcctttctccctcccctct |
| | | ccatccccttttccctaattcccttcctgccttcatctccttccttctctccatcatcactgtctctttga |
| | | gcagctgtgcatctgtttaatcacaagcccactgagttatcaaatgctgctctttacactttaaaag |
| | | agaataaaactcctgagtactggataagaaagtaactaatggtcatttccatctactccactccta |
| | | gcccttttctaacctataccattggtttccaatgtggaggcctaggaatcaggtcatgagtggggt |
| | | gaagaaagcaaaaggatgaaatagactgtgagattgttgtctatgaagcattgtctatagactga |
| | | gtgatgtcttacacatgtattttctcttaaaaaatatgatgatgctgttgtaattgttaaaatccataat |
| | | catttcaaatgtttctacctggagagttgtttaaaaatgcattttaacacacgactgctattatgtgag |
| | | aacctgcgaaatgctttcaagctatcaaggttcattcatgtttgaaatggtttttaaaaatatatgga |
| | | gtcttctgtattctgacatgggcaattttttatgtgtaggtgatgatctgaatagagggcaggctttg |
| | | tatgtcttcaagacatcagtatccatttgccctctgtagaaatcaatgtgttgcttttgatttagagaa |
| | | attctctatcgagacaagctgggaaatgaagaaagccctagatacctcttctatttcatgttttagg |
| | | catttatgcagctctttgctttataaacctgtcacagagataaagtgtgagaacttatagtagcttat |
| | | cggaatcatttatttttctttacccttctaaagaagaactacataattgtatgtacatatatatgtcaaa |
| | | tttattatactcgaaagagaagacattccatttaactactgtctgtacagagctgtttataggttgaa |
| | | aaagaacttgctacaaatctgccgaaaaatagaatgaattctttttaaacatatctgcatctcacat |
| | | aactagattccagtgatggtcattaatatgagtgacacaagggcatttcaatagaatgtattattat |
| | | gtgtataattgctcattcatggactggtggacaagcctctttcagatgatttctgtacctgtaatcaa |
| | | tatgtctgtcttttcagagcatcttggacatttttagcaggggaatcctggaaagtgaagcatttcc |
| | | ataggatttatatagatattttttaagacagggtcttgctctgtcacccaagctggagcgtggctca |
| | | agtgatagtcccacctcagcctcctgagtagctgggactacaggcatgtgccaccatgcctggc |
| | | taactttttaattttttgtagagacagtgtctcactatggtgcccaagctagtcttgcactcctgggct |
| | | caagcagatactcctgccctggcctcccaaagtgctgggattacaggcatgagccactgcacc |
| | | cagacacatattttcatcataagagcgatagttgaagcttttcaacctcatatgctattaggtagtat |
| | | atattgatatttggaactcagaattgttgttaatttttttattctaaaaattattgggactatcattaatta |
| | | actatagctacctagaaatcctagtctttggtggtgaaatcactttgttttctcaacataatgaagttt |
| | | acctgatatttcctcacaaaggagaggttaccggttggagacttatggagaatcctagcatcttga |
| | | tacgtggagaggaaaagatggcgaaaaggcttaaatttgatgtatattttgtgtacagagtaggg |
| | | tattttgcctccttaatggaaacttcacgctagaatgacttctgaggctattctgaaattgttgcattt |
| | | gataatcctttttaatggcaagcctccaaatgtatggtgctttgtcactaaatccttatctatataatttt |
| | | tgaggagacatttcaataacagtttcttttaaaagttctaaggtaggtttttagaagcgttcctttttaa |
| | | aaatcagtttttagctcatgaaactgaaataccagttttaaaaagtcaaggttgaactgaaaggca |
| | | tcattataatagaggagctttgctacattacctgaattattgtttccttctttgcaccaagaacagatt |
| | | aaatatgcattctcttgatatccagtatttgttagcaattttcagagtaaagtattgctatttgatttgttt |
| | | ttctattcataatcatttgcatttgctaagagagtgccttaggtcttttagatctactacactgagtgg |
| | | ctcatgcttacagacgaaagatgattctgcttctttttgtcatccatttgacacccagggccgtctat |
| | | gtgttaaaatgtagcaatagcaatttctcttcctggcttccatgttaatgtggcatgtgcccccagg |
| | | ttttgcttgtatttggagctccatctgatgtgtcaattccggattaatctaaacctgggacactgagtt |
| | | actgacatctgcagttttagcctaaagaactaaaatttccacctttgtgtttctatgcaaattaatcat |
| | | catccaaaaaattctaagacagcacgttggaatccatgctgcgtgcctgtcctataattgcccata |
| | | tagtcatcccttactctattttgtgtaatgaaattttagctcttgtctattgaacttctcattagtgaaata |
| | | aatcaaatagttaatttatttgctaaacttattgtaagcacaaaaacgacattctccacacccaaag |
| | | aggctgctttcttcaatatttatctttatgtgtttcttttttctactttccatcatatcaatttttaaagggttt |
| | | ttgttgttattctttttctgtttgttgcttttatcatacagaagcagtgcttacattgaattaaactaatata |
| | | ctcatcttttttgttttaatgcgctttgctttcttcttttcccttaaacgatccctttgaatgtctcctgatg |
| | | ctataattttggtaatatctagcacttaaaccaatttgaacaagaaatggtttgtacagaaagttatt |
| | | gtcaataatgccactaataccacaaatctttcatgttcttgattatcttttgaaagtgatgtttgtgtctt |
| | | acatctgctcttctttctcattctgcagaaggatatttcagcatttgctatttttcagtattcattctatttt |
| | | gtcgtgtatgcagattgctctcctctgtgtattattcttcctacagtcagacatgacttagcaatgcc |
| | | gaatagcagacctcggtcacttggctgaccactcttttttttgttaatacctactacatttttaaagtg |
| | | gttcaacttagatacctgacaaaatcaggctgagaaaattgtctgaaggaaaatctcccaaacct |
| | | agcttaattaaaaaaaaaaaaatctcatgaagaaagtatctcttcacaaaccatgtgctgtaccaa |
| | | aatgttaggtttttgtgtttctttgaattaaggcttaataatgattttgcctaattctttagtgatatctcta |
| | | ccattagtgagtcacatgtgattggtcagtttaacatggaaagtaagtttcctgatattatgtatgatt |
| | | aatatcagttataaaaaggtaactaaacagcctgtattttctaagccaaggatgaaagcttctttac |
| | | aatttttccttgaaattgtaatattgaaaatgaaaaaattagtctcaacagaatttccctttaataatat |
| | | acatattactatgtaaatatgtttcagggaaaaattgtaatatttacaaatatatatattggaaaacttt |
| | | tccttgctgtttttcttacaactagtgtctctatggtatcttttctccataggacttagacatcaacctg |
| | | acaacaaattcagccctaagtttggaaatatatcattgttttctttttcatactctttcatctgtgtaact |
| | | ctcctaggaataagtagtcaaagaaactttagaggtgtaaagaaaaatgaagttatttgtaaatttg |
| | | catatgcacacacatacgtacacattatgttttgtagaagaaaaaagagctattatataatttatgaa |
| | | aggagactgtgtcattttaagagaaaaagaaatgtttttgtgatttttctgtgaaattcacctttcaca |
| | | atattcagtgtctaagaaattgcttactgttattttgcatgaaacgagagccgctcattgcagcatat |
| | | tagtttacgatctaccttacctgttttaatctactgtttttattaaaatgagttgggttgctgtttgttttta |
| | | tcactgcatttttatgcaaaagatgcaagttggttttttaaaaagcatttgcagaggatcaatttttaa |
| | | gatgataccttttttggtgcttaaataggttgtcaatggaatcactgaaagtagagcagcatgttttc |
| | | acagaaaaaggaagaaactaacgtcaaaacatttttaaagagttttgcttgctacggcttttctcta |
| | | gcatgtagttgatactaagttttgttgcgataacatggtgaatgtttttttttatgtttattgtttacatag |
| | | ctccatttaggataacggagtctttataaataagagatatttttaaggaagtctttgagatcgtgtct |
| | | atttaatgtttcctctcttttccttatgcccctacag |
| 20 | Human | CAAGTTGATGTGGAAAAATGG |
| | Microexon 4 | |
| 21 | Mouse RyR2 | ggctgtcttctggacctccttcctgattagcagaataccggtggggctgcttcattaactgagatt |
| | Intron 3-4 | gcttttgtgcactgaggtgaactgctggtaatgaaaagttggaacactgtcgtgtgttctttaaag |
| | (microexon 4 | ggaagtggttttcgggtctgggggtcacctttagttgacttaggcagccatcggaaacattgcta |
| | capitalized) | ggaactttgttttgttttgttttttaaaatttttttcggctttttcttttcacagCAGGTGGATG |
| | | TGGAAAAGTGGgtatgtgttcccctgcatttacaaaggacttttgtggctgaaaagcg |
| | | catgctcgcagctgcctctgagctttttaattttgcttcctagcatagcttcctttagtgtttgaactaa |
| | | ctgctcggctacaatgaatacatttcacaggtcaccactcagcaccttttgtgtctctgaattatcat |
| | | caccatcaacaggctccttcaaaggactctccactcgaaaaccagattgttttta |
| 22 | Mouse | CAGGTGGATGTGGAAAAGTGG |
| | Microexon 4 | |
| 23 | Mouse RyR2 | PDLSICTFVL EQSLSVRALQ EMLANTVEKS EGQVDVEKWK |
| | Exon 3 | FMMKTAQGGG H |
| | (partial)- | |
| | Exon 6 | |
| | (partial) | |
| 24 | Rat RyR2 Exon | PDLSICTFVL EQSLSVRALQ EMLANTVEKS EGQVDVEKWK |
| | 3 (partial)- | FMMKTAQGGG H |
| | Exon 6 | |
| | (partial) | |
| 25 | Rhesus Monkey | PDLSICTFVL EQSLSVRALQ EMLANTVEKS EGQVDVEKWK |
| | RyR2 Exon 3 | FMMKTAQGGGH |
| | (partial)-Exon | |
| | 6 (partial) | |
| 26 | Rabbit RyR2 | PDLSICTFVL EQSLSVRALQ EMLANTVEKS EGQVDVEKWK |
| | Exon 3 | FMMKTAQGGGH |
| | (partial)- | |
| | Exon 6 | |
| | (partial) | |
| 27 | Chimpanzee | PDLSICTFVL EQSLSVRALQ EMLANTVEKS EGQVDVEKWK |
| | RyR2 Exon 3 | FMMKTAQGGGH |
| | (partial)-Exon | |
| | 6 (partial) | |
| 29 | Pig RyR2 Exon | PDLSICTFVL EQSXSVRALQ EMLANTVEKS EGKVDVEKWK |
| | 3 (partial)- | FMMKTAQGGGH |
| | Exon 6 | |
| | (partial) | |
| 29 | Dog RyR2 Exon | PDLSICTFVL EQSLSVRALQ EMLANTVEKS EGQVDVEKWK |
| | 3 (partial)- | FMMKTAQGGG H |
| | Exon 6 | |
| | (partial) | |
| 30 | Human RyR2 | PDLSICTFVL EQSLSVRALQ EMLANTVEKS EGQVDVEKWK |
| | Exon 3 | FMMKTAQGGGH |
| | (partial)- | |
| | Exon 6 | |
| | (partial) | |
| 31 | Sheep RyR2 | PDLSICTFVL EQSLSVRALQ EMLANTVEKS EGKVDVEKWK |
| | Exon 3 | FMMKTAQGGGH |
| | (partial)- | |
| | Exon 6 | |
| | (partial) | |
| 32 | Cattle RyR2 | PDLSICTFVL EQSLSVRALQ EMLANTVEKS EGKVDVEKWK |
| | Exon 3 | FMMKTAQGGG H |
| | (partial)- | |
| | Exon 6 | |
| | (partial) | |
| 33 | Horse RyR2 | PDLSICTFVL EQSLSVRALQ EMLANTVEKS EGKFMMKTAQ |
| | Exon 3 | GGSH |
| | (partial)- | |
| | Exon 6 | |
| | (partial) | |
| 34 | mRYR2e4F | CCGGACCTGTCTATCTGCAC |
| 35 | mRYR2e4R | CTGTAGGAATGGCGTAGCAA |
| 36 | rRYR2e4F | GACCTGTCCATCTGCACCTT |
| 37 | rRYR2e4R | ACCACTGTAGGAATGGCGTAG |
| 38 | hRYR2e4F | CCAGACCTCTCCATCTGCAC |
| 39 | hRYR2e4R | ATAGGAATGGCGCAGCAATA |
| 40 | mRYR2e75F | CAGGACAGAAGACCCCTCAG |
| 41 | mRYR2e75R | GGCCACAACAGCTCTTTTTC |
| 42 | rRYR2e75F | CAGGTGGCAGATGGCTCTAT |
| 43 | rRYR2e75R | GATTGTACAAAGGGGCCATC |
| 44 | hRYR2e75F | GATGGCAAATGGCTCTTTACA |
| 45 | hRYR2e75R | CCTTTTCCTCTGCTTGGACA |

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

What is claimed:

1. A composition comprising at least one antisense splice switching oligonucleotide (SSO), the at least one antisense SSO comprising a sequence selected from SEQ ID NOs: 1, 4, 15, or 16, or a sequence having at least 90% identity thereto.

2. The composition of claim 1, wherein the at least one antisense SSO consists of a sequence selected from SEQ ID NOs: 1, 4, 15, or 16, or a sequence having at least 90% identity thereto.

3. The composition of claim 1, wherein the at least one antisense SSO is a morpholino.

4. The composition of claim 1, wherein the at least one antisense SSO comprises a phosphorothioate backbone.

5. The composition of claim 1, wherein the at least one antisense SSO comprises at least one modified nucleotide selected from a 2′-O-methyl modified ribose (2′-OMe), a 2′-O-methoxy ethyl modified ribose (2′-MOE), a locked nucleic acid (LNA), and a lipid modified nucleotide.

6. An adenosine-associated virus (AAV) vector comprising an antisense SSO comprising a sequence selected from SEQ ID NOs: 1, 4, 15, or 16, or a sequence having at least 90% identity thereto.

7. The AAV vector of claim 6, wherein the antisense SSO consists of a sequence selected from SEQ ID NOs: 1, 4, 15, or 16, or a sequence having at least 90% identity thereto.

8. The AAV vector of claim 6, wherein the vector is an AAV9 vector.

9. A pharmaceutical composition comprising the antisense SSO of claim 1, and a pharmaceutically acceptable carrier.

10. A method for modulating ryanodine receptor 2 (RyR2) channel activity in a subject in need thereof, the method comprising administering to the subject a pharmaceutical composition comprising an agent that reduces expression of Ryr2 microexon 4.

11. The method of claim 10, wherein the agent comprises at least one antisense splice switching oligonucleotide (SSO) comprising a sequence selected from SEQ ID NOs: 1, 4, 15, or 16, or a sequence having at least 90% identity thereto.

12. The method of claim 11, wherein the at least one antisense SSO comprises a sequence selected from SEQ ID NOs: 4, 15, or 16, or a sequence having at least 90% identity thereto.

13. The method of claim 12, wherein the at least one antisense SSO consists of a sequence selected from SEQ ID NOs: 4, 15, or 16, or a sequence having at least 90% identity thereto.

14. The method of claim 10, wherein the at least one antisense SSO is a morpholino.

15. The method of claim 10, wherein the at least one antisense SSO comprises a phosphorothioate backbone.

16. The method of claim 10, wherein the at least one antisense SSO comprises at least one modified nucleotide selected from a 2′-O-methyl modified ribose (2′-OMe), a 2′-O-methoxy ethyl modified ribose (2′-MOE), a locked nucleic acid (LNA), and a lipid modified nucleotide.

17. The method of claim 10, wherein the at least one antisense SSO is packaged in an AAV vector.

18. The method of claim 17, wherein the AAV vector is an AAV9 vector.

19. The method of claim 10, wherein the subject has a disorder selected from a heart condition.

20. The method of claim 19, wherein the heart condition is selected form an arrythmia, heart failure, diabetic heart disease, atrial fibrillation, catecholaminergic polymorphic ventricular tachycardia, arrhythmogenic right ventricular dysplasia type 2, and a cardiomyopathy.
